SEQUENCE LISTING (254 sequences)

SEQ ID NO: 1 - DNA, 4812 bp, Homo sapiens
  StartCodon (15)..(17)
  StopCodon (3294)..(3296)
gcgctccagacaagatggcgcggccggtccggggagggctcggggccccgcgccgctcgccttgccttctccttctctggctgcttttgcttcggctggagccggtgaccgccgcggccggcccgcgggcgccctgcgcggccgcctgcacttgcgctggggactcgctggactgcggtgggcgcgggctggctgcgttgcccggggacctgccctcctggacgcggagcctaaacctgagttacaacaaactctctgagattgaccctgctggttttgaggacttgccgaacctacaggaagtgtacctcaataataatgagttgacagcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccatggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacttgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcaccattgctgtcgtgagcagcatcgtcctgacgtcactggtctgggtgtgcatcatctaccagaccaggaagaagagtgaagagtacagtgtcaccaacacagatgaaaccgtcgtgccaccagatgttccaagctacctctcttctcaggggaccctttctgaccgacaagaaaccgtggtcaggaccgagggtggccctcaggccaatgggcacattgagagcaatggtgtgtgtccaagagatgcaagccactttccagagcccgacactcacagcgttgcctgcaggcagccaaagctctgtgctgggtctgcgtatcacaaagagccgtggaaagcgatggagaaagctgaagggacacctgggccacataagatggaacacggtggccgggtcgtatgcagtgactgcaacaccgaagtggactgttactccaggggacaagccttccacccccagcctgtgtccagagacagcgcacagccaagtgcgccaaatggcccggagccgggtgggagtgaccaagagcattctccacatcaccagtgcagcaggactgccgctgggtcctgccccgagtgccaagggtcgctctaccccagtaaccacgatagaatgctgacggctgtgaagaaaaagccaatggcatctctagatgggaaaggggattcttcctggactttagcaaggttgtatcacccggactccacagagctacagcctgcatcttcattaacttcaggcagtccagagcgcgcggaagcccagtacttgcttgtttccaatggccacctccccaaagcatgtgacgccagtcccgagtccacgccactgacaggacagctccccgggaaacagagggtgccactgctgttggcaccaaaaagctaggttttgtctacctcagttcttgtcataccaatctctacgggaaagagaggtaggagaggctgcgaggaagcttgggttcaagcgtcactcatctgtacatagttgtaactcccatgtggagtatcagtcgctcacaggacttggatctgaagcacagtaaacgcaagaggggatttgtgtacaaaaggcaaaaaaagtatttgatatcattgtacataagagttttcagagatttcatatatatcttttacagaggctattttaatctttagtgcatggttaacagaaaaaaattatacaattttgacaatattatttttcgtatcaggttgctgtttaattttggagggggtggggaaatagttctggtgccttaacgcatggctggaatttatagaggctacaaccacatttgttcacaggagtttttggtgcggggtgggaaggatggaaggccttggatttatattgcacttcatagacccctaggctgctgtgcggtgggactccacatgcgccggaaggagcttcaggtgagcactgctcatgtgtggatgcccctgcaacaggcttccctgtctgtagagccaggggtgcaagtgccatccacacttgcagtgaatggcttttccttttaggtttaagtcctgtctgtctgtaaggcgtagaatctgtccgtctgtaaggcgtagaatgagggttgttaatccatcacaagcaaaaggtcagaacagttaaacactgcctttcctcctcctcttattttatgataaaagcaaatgtggccttctcagtatcattcgattgctatttgagacttttaaattaaggtaaaggctgctggtgttggtacctgtggatttttctatactgatgttttcgttttgccaatataatgagtattacattggccttgggggacagaaaggaggaagttctgacttttcagggctaccttatttctactaaggacccagagcaggcctgtccatgccattccttcgcacagatgaaactgagctgggactggaaaggacagcccttgacctgggttctgggtataatttgcacttttgagactggtagctaaccatcttatgagtgccaatgtgtcatttagtaaaacttaaatagaaacaaggtccttcaaatgttcctttggccaaaagctgaagggagttactgagaaaatagttaacaattactgtcaggtgtcatcactgttcaaaaggtaagcacatttagaattttgttcttgacagttaactgactaatcttacttccacaaaatatgtgaatttgctgcttctgagaggcaatgtgaaagagggagtattacttttatgtacaaagttatttatttatagaaattttggtacagtgtacattgaaaaccatgtaaaatattgaagtgtctaacaaatggcattgaagtgtctttaataaaggttcatttataaatgtcaaaaaaaaaaaaaaaaaaaaaaaaaaaaaaaaaaaaaaaaaaaaaaaaaaa

SEQ ID NO: 2 - protein, 1093 aa, Homo sapiens
MARPVRGGLGAPRRSPCLLLLWLLLLRLEPVTAAAGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTMARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFTIAVVSSIVLTSLVWVCIIYQTRKKSEEYSVTNTDETVVPPDVPSYLSSQGTLSDRQETVVRTEGGPQANGHIESNGVCPRDASHFPEPDTHSVACRQPKLCAGSAYHKEPWKAMEKAEGTPGPHKMEHGGRVVCSDCNTEVDCYSRGQAFHPQPVSRDSAQPSAPNGPEPGGSDQEHSPHHQCSRTAAGSCPECQGSLYPSNHDRMLTAVKKKPMASLDGKGDSSWTLARLYHPDSTELQPASSLTSGSPERAEAQYLLVSNGHLPKACDASPESTPLTGQLPGKQRVPLLLAPKS

SEQ ID NO: 3 - DNA, 4774 bp, Homo sapiens
  StartCodon (142)..(144)
  StopCodon (1075)..(1077)
gggggcgggtgcctggagcacggcgctggggccgcccgcagcgctcactcgctcgcactcagtcgcgggaggcttccccgcgccggccgcgtcccgcccgctccccggcaccagaagttcctctgcgcgtccgacggcgacatgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctagcccagctgggggacagtgggctgttgtggctgggtctggggcaggtgcatttgagccagggctggctctgtgagtggcctccttggcctcggccctggttccctccctcctgctctgggctcagatactgtgacatcccagaagcccagcccctcaacccctctggatgctacatggggatgctggacggctcagcccctgttccaaggattttggggtgctgagattctcccctagagacctgaaattcaccagctacagatgccaaatgacttacatcttaagaagtctcagaacgtccagcccttcagcagctctcgttctgagacatgagccttgggatgtggcagcatcagtgggacaagatggacactgggccaccctcccaggcaccagacacagggcacggtggagagacttctcccccgtggccgccttggctcccccgttttgcccgaggctgctcttctgtcagacttcctctttgtaccacagtggctctggggccaggcctgcctgcccactggccatcgccaccttccccagctgcctcctaccagcagtttctctgaagatctgtcaacaggttaagtcaatctggggcttccactgcctgcattccagtccccagagcttggtggtcccgaaacgggaagtacatattggggcatggtggcctccgtgagcaaatggtgtcttgggcaatctgaggccaggacagatgttgccccacccactggagatggtgctgagggaggtgggtggggccttctgggaaggtgagtggagaggggcacctgccccccgccctccccatcccctactcccactgctcagcgcgggccattgcaagggtgccacacaatgtcttgtccaccctgggacacttctgagtatgaagcgggatgctattaaaaactacatggggaaacaggtgcaaaccctggagatggattgtaagagccagtttaaatctgcactctgctgctcctcccccacccccaccttccactccatacaatctgggcctggtggagtcttcgcttcagagccattcggccaggtgcgggtgatgttcccatctcctgcttgtgggcatgccctggctttgtttttatacacataggcaaggtgagtcctctgtggaattgtgattgaaggattttaaagcaggggaggagagtagggggcatctctgtacactctgggggtaaaacagggaaggcagtgcctgagcatggggacaggtgaggtggggctgggcagaccccctgtagcgtttagcaggatgggggccccaggtactgtggagagcatagtccagcctgggcatttgtctcctagcagcctacactggctctgctgagctgggcctgggtgctgaaagccaggatttggggctaggcgggaagatgttcgcccaattgcttggggggttggggggatggaaaaggggagcacctctaggctgcctggcagcagtgagccctgggcctgtggctacagccagggaaccccacctggacacatggccctgcttctaagccccccagttaggcccaaaggaatggtccactgagggcctcctgctctgcctgggctgggccaggggctttgaggagagggtaaacataggcccggagatggggctgacacctcgagtggccagaatatgcccaaaccccggcttctcccttgtccctaggcagaggggggtcccttcttttgttccctctggtcaccacaatgcttgatgccagctgccataggaagagggtgctggctggccatggtggcacacacctgtcctcccagcactttgcagggctgaggtggaaggaccgcttaagcccaggtgttcaaggctgctgtgagctgtgttcgagccactacactccagcctggggacggagcaaaactttgcctcaaaacaaattttaaaaagaaagaaagaaggaaagagggtatgtttttcacaattcatgggggcctgcatggcaggagtggggacaggacacctgctgttcctggagtcgaaggacaagcccacagcccagattccggttctcccaactcaggaagagcatgccctgccctctggggaggctggcctggccccagccctcagctgctgaccttgaggcagagacaacttctaagaatttggctgccagaccccaggcctggctgctgctgtgtggagagggaggcggcccgcagcagaacagccaccgcacttcctcctcagcttcctctggtgcggccctgccctctcttctctggacccttttacaactgaacgcatctgggcttcgtggtttcctgttttcagcgaaatttactctgagctcccagttccatcttcatccatggccacaggccctgcctacaacgcactagggacgtccctccctgctgctgctggggaggggcaggctgctggagccgccctctgagttgcccgggatggtagtgcctctgatgccagccctggtggctgtgggctggggtgcatgggagagctgggtgcgagaacatggcgcctccagggggcgggaggagcactaggggctggggcaggaggctcctggagcgctggattcgtggcacagtctgaggccctgagagggaaatccatgcttttaagaactaattcattgttaggagatcaatcaggaattaggggccatcttacctatctcctgacattcacagtttaatagagacttcctgcctttattccctcccagggagaggctgaaggaatggaattgaaagcaccatttggagggttttgctgacacagcggggactgctcagcactccctaaaaacacaccatggaggccactggtgactgctggtgggcaggctggccctgcctgggggagtccgtggcgatgggcgctggggtggaggtgcaggagccccaggacctgcttttcaaaagacttctgcctgaccagagctcccactacatgcagtggcccagggcagaggggctgatacatggcctttttcagggggtgctcctcgcggggtggacttgggagtgtgcagtgggacagggggctgcaggggtcctgccaccaccgagcaccaacttggcccctggggtcctgcctcatgaatgaggccttccccagggctggcctgactgtgctgggggctgggttaacgttttctcagggaaccacaatgcacgaaagaggaactggggttgctaaccaggatgctgggaacaaaggcctcttgaagcccagccacagcccagctgagcatgaggcccagcccatagacggcacaggccacctggcccattccctgggcattccctgctttgcattgctgcttctcttcaccccatggaggctatgtcaccctaactatcctggaatgtgttgagagggattctgaatgatcaatatagcttggtgagacagtgccgagatagatagccatgtctgccttgggcacgggagagggaagtggcagcatgcatgctgtttcttggccttttctgttagaatacttggtgctttccaacacactttcacatgtgttgtaacttgtttgatccacccccttccctgaaaatcctgggaggttttattgctgccatttaacacagagggcaatagaggttctgaaaggtctgtgtcttgtcaaaacaagtaaacggtggaactacgactaaa

SEQ ID NO: 4 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 5 - protein, 20 aa, Artificial Sequence
  LRIG Peptide 1
AGPRAPCAAACTCAGDSLDC

SEQ ID NO: 6 - protein, 20 aa, Artificial Sequence
  LRIG Peptide 2
CTCAGDSLDCGGRGLAALPG

SEQ ID NO: 7 - protein, 20 aa, Artificial Sequence
  LRIG Peptide 3
GGRGLAALPGDLPSWTRSLN

SEQ ID NO: 8 - protein, 20 aa, Artificial Sequence
  LRIG Peptide 4
DLPSWTRSLNLSYNKLSEID

SEQ ID NO: 9 - protein, 20 aa, Artificial Sequence
  LRIG Peptide 5
LSYNKLSEIDPAGFEDLPNL

SEQ ID NO: 10 - protein, 20 aa, Artificial Sequence
  LRIG Peptide 6
PAGFEDLPNLQEVYLNNNEL

SEQ ID NO: 11 - protein, 20 aa, Artificial Sequence
  LRIG Peptide 7
QEVYLNNNELTAVPSLGAAS

SEQ ID NO: 12 - protein, 20 aa, Artificial Sequence
  LRIG Peptide 8
TAVPSLGAASSHVVSLFLQH

SEQ ID NO: 13 - protein, 20 aa, Artificial Sequence
  LRIG Peptide 9
SHVVSLFLQHNKIRSVEGSQ

SEQ ID NO: 14 - protein, 20 aa, Artificial Sequence
  LRIG Peptide 10
NKIRSVEGSQLKAYLSLEVL

SEQ ID NO: 15 - protein, 20 aa, Artificial Sequence
  LRIG Peptide 11
LKAYLSLEVLDLSLNNITEV

SEQ ID NO: 16 - protein, 20 aa, Artificial Sequence
  LRIG Peptide 12
DLSLNNITEVRNTCFPHGPP

SEQ ID NO: 17 - protein, 20 aa, Artificial Sequence
  LRIG Peptide 13
RNTCFPHGPPIKELNLAGNR

SEQ ID NO: 18 - protein, 20 aa, Artificial Sequence
  LRIG Peptide 14
IKELNLAGNRIGTLELGAFD

SEQ ID NO: 19 - protein, 20 aa, Artificial Sequence
  LRIG Peptide 15
IGTLELGAFDGLSRSLLTLR

SEQ ID NO: 20 - protein, 20 aa, Artificial Sequence
  LRIG Peptide 16
GLSRSLLTLRLSKNRITQLP

SEQ ID NO: 21 - protein, 20 aa, Artificial Sequence
  LRIG Peptide 17
LSKNRITQLPVRAFKLPRLT

SEQ ID NO: 22 - protein, 20 aa, Artificial Sequence
  LRIG Peptide 18
VRAFKLPRLTQLDLNRNRIR

SEQ ID NO: 23 - protein, 20 aa, Artificial Sequence
  LRIG Peptide 19
QLDLNRNRIRLIEGLTFQGL

SEQ ID NO: 24 - protein, 20 aa, Artificial Sequence
  LRIG Peptide 20
LIEGLTFQGLNSLEVLKLQR

SEQ ID NO: 25 - protein, 20 aa, Artificial Sequence
  LRIG Peptide 21
NSLEVLKLQRNNISKLTDGA

SEQ ID NO: 26 - protein, 20 aa, Artificial Sequence
  LRIG Peptide 22
NNISKLTDGAFWGLSKMHVL

SEQ ID NO: 27 - protein, 20 aa, Artificial Sequence
  LRIG Peptide 23
FWGLSKMHVLHLEYNSLVEV

SEQ ID NO: 28 - protein, 20 aa, Artificial Sequence
  LRIG Peptide 24
HLEYNSLVEVNSGSLYGLTA

SEQ ID NO: 29 - protein, 20 aa, Artificial Sequence
  LRIG Peptide 25
NSGSLYGLTALHQLHLSNNS

SEQ ID NO: 30 - protein, 20 aa, Artificial Sequence
  LRIG Peptide 26
LHQLHLSNNSIARIHRKGWS

SEQ ID NO: 31 - protein, 20 aa, Artificial Sequence
  LRIG Peptide 27
IARIHRKGWSFCQKLHELVL

SEQ ID NO: 32 - protein, 20 aa, Artificial Sequence
  LRIG Peptide 28
FCQKLHELVLSFNNLTRLDE

SEQ ID NO: 33 - protein, 20 aa, Artificial Sequence
  LRIG Peptide 29
SFNNLTRLDEESLAELSSLS

SEQ ID NO: 34 - protein, 20 aa, Artificial Sequence
  LRIG Peptide 30
ESLAELSSLSVLRLSHNSIS

SEQ ID NO: 35 - protein, 20 aa, Artificial Sequence
  LRIG Peptide 31
VLRLSHNSISHIAEGAFKGL

SEQ ID NO: 36 - protein, 20 aa, Artificial Sequence
  LRIG Peptide 32
HIAEGAFKGLRSLRVLDLDH

SEQ ID NO: 37 - protein, 20 aa, Artificial Sequence
  LRIG Peptide 33
RSLRVLDLDHNEISGTIEDT

SEQ ID NO: 38 - protein, 20 aa, Artificial Sequence
  LRIG Peptide 34
NEISGTIEDTSGAFSGLDSL

SEQ ID NO: 39 - protein, 20 aa, Artificial Sequence
  LRIG Peptide 35
SGAFSGLDSLSKLTLFGNKI

SEQ ID NO: 40 - protein, 20 aa, Artificial Sequence
  LRIG Peptide 36
SKLTLFGNKIKSVAKRAFSG

SEQ ID NO: 41 - protein, 20 aa, Artificial Sequence
  LRIG Peptide 37
KSVAKRAFSGLEGLEHLNLG

SEQ ID NO: 42 - protein, 20 aa, Artificial Sequence
  LRIG Peptide 38
LEGLEHLNLGGNAIRSVQFD

SEQ ID NO: 43 - protein, 20 aa, Artificial Sequence
  LRIG Peptide 39
GNAIRSVQFDAFVKMKNLKE

SEQ ID NO: 44 - protein, 20 aa, Artificial Sequence
  LRIG Peptide 40
AFVKMKNLKELHISSDSFLC

SEQ ID NO: 45 - protein, 20 aa, Artificial Sequence
  LRIG Peptide 41
LHISSDSFLCDCQLKWLPPW

SEQ ID NO: 46 - protein, 20 aa, Artificial Sequence
  LRIG Peptide 42
DCQLKWLPPWLIGRMLQAFV

SEQ ID NO: 47 - protein, 20 aa, Artificial Sequence
  LRIG Peptide 43
LIGRMLQAFVTATCAHPESL

SEQ ID NO: 48 - protein, 20 aa, Artificial Sequence
  LRIG Peptide 44
TATCAHPESLKGQSIFSVPP

SEQ ID NO: 49 - protein, 20 aa, Artificial Sequence
  LRIG Peptide 45
KGQSIFSVPPESFVCDDFLK

SEQ ID NO: 50 - protein, 20 aa, Artificial Sequence
  LRIG Peptide 46
ESFVCDDFLKPQIITQPETT

SEQ ID NO: 51 - protein, 20 aa, Artificial Sequence
  LRIG Peptide 47
PQIITQPETTMAMVGKDIRF

SEQ ID NO: 52 - protein, 20 aa, Artificial Sequence
  LRIG Peptide 48
MAMVGKDIRFTCSAASSSSS

SEQ ID NO: 53 - protein, 20 aa, Artificial Sequence
  LRIG Peptide 49
TCSAASSSSSPMTFAWKKDN

SEQ ID NO: 54 - protein, 20 aa, Artificial Sequence
  LRIG Peptide 50
PMTFAWKKDNEVLTNADMEN

SEQ ID NO: 55 - protein, 20 aa, Artificial Sequence
  LRIG Peptide 51
EVLTNADMENFVHVHAQDGE

SEQ ID NO: 56 - protein, 20 aa, Artificial Sequence
  LRIG Peptide 52
FVHVHAQDGEVMEYTTILHL

SEQ ID NO: 57 - protein, 20 aa, Artificial Sequence
  LRIG Peptide 53
VMEYTTILHLRQVTFGHEGR

SEQ ID NO: 58 - protein, 20 aa, Artificial Sequence
  LRIG Peptide 54
RQVTFGHEGRYQCVITNHFG

SEQ ID NO: 59 - protein, 20 aa, Artificial Sequence
  LRIG Peptide 55
YQCVITNHFGSTYSHKARLT

SEQ ID NO: 60 - protein, 20 aa, Artificial Sequence
  LRIG Peptide 56
STYSHKARLTVNVLPSFTKT

SEQ ID NO: 61 - protein, 20 aa, Artificial Sequence
  LRIG Peptide 57
VNVLPSFTKTPHDITIRTTT

SEQ ID NO: 62 - protein, 20 aa, Artificial Sequence
  LRIG Peptide 58
PHDITIRTTTMARLECAATG

SEQ ID NO: 63 - protein, 20 aa, Artificial Sequence
  LRIG Peptide 59
MARLECAATGHPNPQIAWQK

SEQ ID NO: 64 - protein, 20 aa, Artificial Sequence
  LRIG Peptide 60
HPNPQIAWQKDGGTDFPAAR

SEQ ID NO: 65 - protein, 20 aa, Artificial Sequence
  LRIG Peptide 61
DGGTDFPAARERRMHVMPDD

SEQ ID NO: 66 - protein, 20 aa, Artificial Sequence
  LRIG Peptide 62
ERRMHVMPDDDVFFITDVKI

SEQ ID NO: 67 - protein, 20 aa, Artificial Sequence
  LRIG Peptide 63
DVFFITDVKIDDAGVYSCTA

SEQ ID NO: 68 - protein, 20 aa, Artificial Sequence
  LRIG Peptide 64
DDAGVYSCTAQNSAGSISAN

SEQ ID NO: 69 - protein, 20 aa, Artificial Sequence
  LRIG Peptide 65
QNSAGSISANATLTVLETPS

SEQ ID NO: 70 - protein, 20 aa, Artificial Sequence
  LRIG Peptide 66
ATLTVLETPSLVVPLEDRVV

SEQ ID NO: 71 - protein, 20 aa, Artificial Sequence
  LRIG Peptide 67
LVVPLEDRVVSVGETVALQC

SEQ ID NO: 72 - protein, 20 aa, Artificial Sequence
  LRIG Peptide 68
SVGETVALQCKATGNPPPRI

SEQ ID NO: 73 - protein, 20 aa, Artificial Sequence
  LRIG Peptide 69
KATGNPPPRITWFKGDRPLS

SEQ ID NO: 74 - protein, 20 aa, Artificial Sequence
  LRIG Peptide 70
TWFKGDRPLSLTERHHLTPD

SEQ ID NO: 75 - protein, 20 aa, Artificial Sequence
  LRIG Peptide 71
LTERHHLTPDNQLLVVQNVV

SEQ ID NO: 76 - protein, 20 aa, Artificial Sequence
  LRIG Peptide 72
NQLLVVQNVVAEDAGRYTCE

SEQ ID NO: 77 - protein, 20 aa, Artificial Sequence
  LRIG Peptide 73
AEDAGRYTCEMSNTLGTERA

SEQ ID NO: 78 - protein, 20 aa, Artificial Sequence
  LRIG Peptide 74
MSNTLGTERAHSQLSVLPAA

SEQ ID NO: 79 - protein, 20 aa, Artificial Sequence
  LRIG Peptide 75
HSQLSVLPAAGCRKDGTTVG

SEQ ID NO: 80 - protein, 10 aa, Artificial Sequence
  LRIG Peptide 76
GCRKDGTTVG

SEQ ID NO: 81 - protein, 7 aa, Artificial Sequence
  CDR1-H
GYTFTSY

SEQ ID NO: 82 - protein, 12 aa, Artificial Sequence
  CDR2-H
WIYPGNVNTKYN

SEQ ID NO: 83 - protein, 8 aa, Artificial Sequence
  CDR3-H
EELGGFAY

SEQ ID NO: 84 - protein, 8 aa, Artificial Sequence
  CDR4-H
GYTFTSFW

SEQ ID NO: 85 - protein, 8 aa, Artificial Sequence
  CDR5-H
IYPGSSSA

SEQ ID NO: 86 - protein, 11 aa, Artificial Sequence
  CDR6-H
ARSSYRYSYYS

SEQ ID NO: 87 - protein, 8 aa, Artificial Sequence
  CDR7-H
GFSLTSYG

SEQ ID NO: 88 - protein, 7 aa, Artificial Sequence
  CDR8-H
IWSGGST

SEQ ID NO: 89 - protein, 16 aa, Artificial Sequence
  CDR9-H
ARNWAYYSNHYYVMDY

SEQ ID NO: 90 - protein, 8 aa, Artificial Sequence
  CDR10-H
GFTFSASA

SEQ ID NO: 91 - protein, 8 aa, Artificial Sequence
  CDR11-H
SDRGSSSD

SEQ ID NO: 92 - protein, 12 aa, Artificial Sequence
  CDR12-H
AGGVSTVINFDY

SEQ ID NO: 93 - protein, 8 aa, Artificial Sequence
  CDR13-H
GYTFTGYW

SEQ ID NO: 94 - protein, 8 aa, Artificial Sequence
  CDR14-H
ILPGSGST

SEQ ID NO: 95 - protein, 15 aa, Artificial Sequence
  CDR15-H
EREVYDNDEGAWFAN

SEQ ID NO: 96 - protein, 8 aa, Artificial Sequence
  CDR16-H
GYNFINSW

SEQ ID NO: 97 - protein, 8 aa, Artificial Sequence
  CDR17-H
FYPGNSDT

SEQ ID NO: 98 - protein, 7 aa, Artificial Sequence
  CDR18-H
TELGGDY

SEQ ID NO: 99 - protein, 9 aa, Artificial Sequence
  CDR19-H
GYSITSGYD

SEQ ID NO: 100 - protein, 7 aa, Artificial Sequence
  CDR20-H
ISYSGYT

SEQ ID NO: 101 - protein, 10 aa, Artificial Sequence
  CDR21-H
ARDSNWYFDV

SEQ ID NO: 102 - protein, 8 aa, Artificial Sequence
  CDR22-H
GFTFSSHA

SEQ ID NO: 103 - protein, 8 aa, Artificial Sequence
  CDR23-H
ISDGGRYT

SEQ ID NO: 104 - protein, 12 aa, Artificial Sequence
  CDR24-H
ARDAYVSTSFAY

SEQ ID NO: 105 - protein, 8 aa, Artificial Sequence
  CDR25-H
GFTFSNYW

SEQ ID NO: 106 - protein, 10 aa, Artificial Sequence
  CDR26-H
IRLKSENYPT

SEQ ID NO: 107 - protein, 9 aa, Artificial Sequence
  CDR27-H
TGRGYSNYA

SEQ ID NO: 108 - protein, 8 aa, Artificial Sequence
  CDR28-H
GHTFSSYW

SEQ ID NO: 109 - protein, 8 aa, Artificial Sequence
  CDR29-H
ILPGKGNT

SEQ ID NO: 110 - protein, 6 aa, Artificial Sequence
  CDR30-H
ARNFDV

SEQ ID NO: 111 - protein, 8 aa, Artificial Sequence
  CDR31-H
GYTFTSYW

SEQ ID NO: 112 - protein, 8 aa, Artificial Sequence
  CDR32-H
IDPSDSYT

SEQ ID NO: 113 - protein, 9 aa, Artificial Sequence
  CDR33-H
ARARVDFDY

SEQ ID NO: 114 - protein, 8 aa, Artificial Sequence
  CDR34-H
GFNIKDDY

SEQ ID NO: 115 - protein, 8 aa, Artificial Sequence
  CDR35-H
IDPENGDT

SEQ ID NO: 116 - protein, 6 aa, Artificial Sequence
  CDR36-H
TTGSTV

SEQ ID NO: 117 - protein, 8 aa, Artificial Sequence
  CDR37-H
GYTFTTYG

SEQ ID NO: 118 - protein, 8 aa, Artificial Sequence
  CDR38-H
INTYSGVP

SEQ ID NO: 119 - protein, 8 aa, Artificial Sequence
  CDR39-H
ALTGVLEN

SEQ ID NO: 120 - protein, 8 aa, Artificial Sequence
  CDR40-H
GYTFTDYY

SEQ ID NO: 121 - protein, 8 aa, Artificial Sequence
  CDR41-H
ISPKNGGT

SEQ ID NO: 122 - protein, 9 aa, Artificial Sequence
  CDR42-H
ASPFYDDYY

SEQ ID NO: 123 - protein, 8 aa, Artificial Sequence
  CDR43-H
GYTFTSYN

SEQ ID NO: 124 - protein, 8 aa, Artificial Sequence
  CDR44-H
IYPGNGYT

SEQ ID NO: 125 - protein, 8 aa, Artificial Sequence
  CDR45-H
ARGVGFDY

SEQ ID NO: 126 - protein, 8 aa, Artificial Sequence
  CDR46-H
GYTFTSYG

SEQ ID NO: 127 - protein, 8 aa, Artificial Sequence
  CDR47-H
IYPGTSST

SEQ ID NO: 128 - protein, 11 aa, Artificial Sequence
  CDR48-H
SRSSYRYTYYS

SEQ ID NO: 129 - protein, 8 aa, Artificial Sequence
  CDR49-H
GYTFTSYD

SEQ ID NO: 130 - protein, 8 aa, Artificial Sequence
  CDR50-H
IYPRDGTT

SEQ ID NO: 131 - protein, 11 aa, Artificial Sequence
  CDR51-H
AREDYGSSFHN

SEQ ID NO: 132 - protein, 8 aa, Artificial Sequence
  CDR52-H
ILPGSGRT

SEQ ID NO: 133 - protein, 15 aa, Artificial Sequence
  CDR53-H
AREIYDGHYEAWYAY

SEQ ID NO: 134 - protein, 8 aa, Artificial Sequence
  CDR54-H
GFTFSSYT

SEQ ID NO: 135 - protein, 11 aa, Artificial Sequence
  CDR55-H
ARGWLLRNFDF

SEQ ID NO: 136 - protein, 8 aa, Artificial Sequence
  CDR56-H
IDPNSGGT

SEQ ID NO: 137 - protein, 11 aa, Artificial Sequence
  CDR57-H
ARLGYGNPFDY

SEQ ID NO: 138 - protein, 8 aa, Artificial Sequence
  CDR58-H
GFKIKDDY

SEQ ID NO: 139 - protein, 8 aa, Artificial Sequence
  CDR59-H
IYPGNSET

SEQ ID NO: 140 - protein, 13 aa, Artificial Sequence
  CDR60-H
TRPLANWDGPFDY

SEQ ID NO: 141 - protein, 10 aa, Artificial Sequence
  CDR61-H
IRLKSDNYAT

SEQ ID NO: 142 - protein, 8 aa, Artificial Sequence
  CDR62-H
IDPEKGDT

SEQ ID NO: 143 - protein, 8 aa, Artificial Sequence
  CDR63-H
GFTFSSYA

SEQ ID NO: 144 - protein, 8 aa, Artificial Sequence
  CDR64-H
ISDGGSYT

SEQ ID NO: 145 - protein, 12 aa, Artificial Sequence
  CDR65-H
SRAGSTVVNFGY

SEQ ID NO: 146 - protein, 11 aa, Artificial Sequence
  CDR1-L
RASQDISNYLS

SEQ ID NO: 147 - protein, 7 aa, Artificial Sequence
  CDR2-L
YTSILHS

SEQ ID NO: 148 - protein, 9 aa, Artificial Sequence
  CDR3-L
QQGNTLPRT

SEQ ID NO: 149 - protein, 11 aa, Artificial Sequence
  CDR4-L
QSIVHSNGNTF

SEQ ID NO: 150 - protein, 3 aa, Artificial Sequence
  CDR5-L
KVS

SEQ ID NO: 151 - protein, 9 aa, Artificial Sequence
  CDR6-L
FQGSHVPLS

SEQ ID NO: 152 - protein, 11 aa, Artificial Sequence
  CDR7-L
QSIVHSNGNTY

SEQ ID NO: 153 - protein, 9 aa, Artificial Sequence
  CDR8-L
FQGSHVPLT

SEQ ID NO: 154 - protein, 11 aa, Artificial Sequence
  CDR9-L
QSLLYSDGKTY

SEQ ID NO: 155 - protein, 3 aa, Artificial Sequence
  CDR10-L
QVS

SEQ ID NO: 156 - protein, 9 aa, Artificial Sequence
  CDR11-L
LQGTYYPHT

SEQ ID NO: 157 - protein, 6 aa, Artificial Sequence
  CDR12-L
ENVDTY

SEQ ID NO: 158 - protein, 3 aa, Artificial Sequence
  CDR13-L
  misc_feature (3)..(3): Xaa can be any naturally occurring amino acid
GAX

SEQ ID NO: 159 - protein, 9 aa, Artificial Sequence
  CDR14-L
LQGHKDPYT

SEQ ID NO: 160 - protein, 3 aa, Artificial Sequence
  CDR15-L
GAS

SEQ ID NO: 161 - protein, 9 aa, Artificial Sequence
  CDR16-L
GQSHNYPYT

SEQ ID NO: 162 - protein, 9 aa, Artificial Sequence
  CDR17-L
FQGSHFPWT

SEQ ID NO: 163 - protein, 7 aa, Artificial Sequence
  CDR18-L
SSISSST

SEQ ID NO: 164 - protein, 3 aa, Artificial Sequence
  CDR19-L
GTS

SEQ ID NO: 165 - protein, 9 aa, Artificial Sequence
  CDR20-L
QQWSGYPLT

SEQ ID NO: 166 - protein, 7 aa, Artificial Sequence
  CDR21-L
STTGSVH

SEQ ID NO: 167 - protein, 9 aa, Artificial Sequence
  CDR22-L
FQGSHVPFT

SEQ ID NO: 168 - protein, 11 aa, Artificial Sequence
  CDR23-L
QSLLHRNGKTY

SEQ ID NO: 169 - protein, 3 aa, Artificial Sequence
  CDR24-L
LVS

SEQ ID NO: 170 - protein, 9 aa, Artificial Sequence
  CDR25-L
FQTTHFPHT

SEQ ID NO: 171 - protein, 11 aa, Artificial Sequence
  CDR26-L
QSIVHSNGNSY

SEQ ID NO: 172 - protein, 11 aa, Artificial Sequence
  CDR27-L
QSIVQSNGITY

SEQ ID NO: 173 - protein, 6 aa, Artificial Sequence
  CDR28-L
EDVDTY

SEQ ID NO: 174 - protein, 9 aa, Artificial Sequence
  CDR29-L
GQSYSYPWT

SEQ ID NO: 175 - protein, 6 aa, Artificial Sequence
  CDR30-L
ENIYSN

SEQ ID NO: 176 - protein, 3 aa, Artificial Sequence
  CDR31-L
TAT

SEQ ID NO: 177 - protein, 9 aa, Artificial Sequence
  CDR32-L
QHFYGSPRT

SEQ ID NO: 178 - protein, 11 aa, Artificial Sequence
  CDR33-L
  misc_feature (7)..(7): Xaa can be any naturally occurring amino acid
QSLLYSXGKTY

SEQ ID NO: 179 - protein, 3 aa, Artificial Sequence
  CDR34-L
  misc_feature (1)..(1): Xaa can be any naturally occurring amino acid
  misc_feature (3)..(3): Xaa can be any naturally occurring amino acid
XVX

SEQ ID NO: 180 - protein, 9 aa, Artificial Sequence
  CDR35-L
VQGIHFPLT

SEQ ID NO: 181 - protein, 6 aa, Artificial Sequence
  CDR36-L
ENVGTY

SEQ ID NO: 182 - protein, 8 aa, Artificial Sequence
  CDR37-L
GQSYTDLT

SEQ ID NO: 183 - protein, 11 aa, Artificial Sequence
  CDR38-L
QNIVHTNGNTY

SEQ ID NO: 184 - protein, 9 aa, Artificial Sequence
  CDR39-L
FQGSHIPWT

SEQ ID NO: 185 - protein, 3 aa, Artificial Sequence
  CDR40-L
LVS

SEQ ID NO: 186 - protein, 9 aa, Artificial Sequence
  CDR41-L
VQGPHFPLT

SEQ ID NO: 187 - protein, 11 aa, Artificial Sequence
  CDR42-L
QNIAHSNGNTY

SEQ ID NO: 188 - protein, 117 aa, Artificial Sequence
  VH-1
QVQLVQSGAEVKKPGASVKVSCKASGYTFTSYYLHWVRQAPGQRLEWMGWIYPGNVNTKYNQKFQGRVTITADKSASTAYMELSSLRSEDTAVYFCAREELGGFAYWGQGTLVTVSS

SEQ ID NO: 189 - protein, 117 aa, Artificial Sequence
  VH-2
QVQLVQSGAEVKKPGASVKVSCKASGYTFTSYYLHWVRQAPGQRLEWIGWIYPGNVNTKYNEKFQGRVTLTADKSASTAYMELSSLRSEDTAVYFCAREELGGFAYWGQGTLVTVSS

SEQ ID NO: 190 - protein, 107 aa, Artificial Sequence
  VL-1
DIQMTQSPSSVSASIGDRVTITCRASQDISNYLSWYQQKPGKAPKLLIYYTSILHSGVPSRFSGSGSGTDYTLTISSLQPEDFATYFCQQGNTLPRTFGGGTKVEIK

SEQ ID NO: 191 - protein, 106 aa, Artificial Sequence
  VL-2
DIQMTQSPSSLSASVGNRVTITCRASQDISNYLSWYQQKPGKVPKLLIYYTSIHSGVPSRFSGSGSGTDYSLTISSLQPEDVATYFCQQGNTLPRTFGQGTKVEIK

SEQ ID NO: 192 - protein, 118 aa, Artificial Sequence
  802.1H3.2A4 VH
QVQLQQPGDELVKPGASVKLSCKASGYTFTSFWIHWVKQRPGQGLEWIGNIYPGSSSANYNEKFKSKATLTVDTSSSTAYMQLSSLTSDDSAVYYCARSSYRYSYYSWGQGTTLTVSS

SEQ ID NO: 193 - protein, 122 aa, Artificial Sequence
  802.2B7.2D9 VH
QVQLKQSGPGLVQPSQSLSITCTVSGFSLTSYGVHWVRQSPGKGLEYLGVIWSGGSTDYNAAFISRLSISKDNSKSQVFFKMNSLRADDTAIYYCARNWAYYSNHYYVMDYWGQGTSVTVSS

SEQ ID NO: 194 - protein, 119 aa, Artificial Sequence
  802.2B7.2F9 VH
EVQLVESGGGLAKPGGSLKLSCAASGFTFSASAMSWVRQTPEKRLEWVATISDGGSYTYYPDNVKGRFTISRDNAKSNLYLQMSHLKSEDTAMYYCAGGVSTVINFDYWGQGTTLTVSS

SEQ ID NO: 195 - protein, 122 aa, Artificial Sequence
  802.2F11.2B6 VH
QVQLQQSGAELMKPGASVKLSCKATGYTFTGYWIEWVKQRPGHGLEWIGEILPGSGSTHYNEKFKGKATFTADTSSNTAYIQLSSLTTEDSAIYYCEREVYDNDEGAWFANWGQGTLVTVSA

SEQ ID NO: 196 - protein, 114 aa, Artificial Sequence
  802.2F4.2A3 VH
QVTLKESGPGILKPSQTLSLTCSFSGFSLSTSGMAVGWIRQPSGKGLEWLAHIWWDDDKYYNPSLKSQLTISKDTSRNQVFLKITSVDTADTATYYCARRAYPYFDVWGAGPRS

SEQ ID NO: 197 - protein, 114 aa, Artificial Sequence
  802.2F4.2C7 VH
EVQLQQSGTVLARPGASVKMSCKASGYNFINSWMHWVKQRPGQGLEWIGAFYPGNSDTRYNQKFMGKAKLTAVTSASTAYMEVSSLTNEDSAVYYCTELGGDYWGQGTTLTVSS

SEQ ID NO: 198 - protein, 117 aa, Artificial Sequence
  802.3B10.2C10 VH
DVQLQESGPGLVKPSLSLSLTCTVTGYSITSGYDWHWIRHFPGNILEWMGYISYSGYTNYNPSLKSRISITHDTSKNHFFLKLNSVTTEDTATYYCARDSNWYFDVWGAGTTVTVSS

SEQ ID NO: 199 - protein, 119 aa, Artificial Sequence
  802.3D4.2D4 VH
EVQLVESGGGLVKPGGSLKLSCAASGFTFSSHAMSWVRQTPEKRLEWVATISDGGRYTYYPDNVKGRFTISRDNAKNNLYLQMSHLKSEDTAIYYCARDAYVSTSFAYWGQGTLVTVSA

SEQ ID NO: 200 - protein, 118 aa, Artificial Sequence
  802.3D5.2G4 VH
EVKLEESGGGLVQPGGSMTLSCVASGFTFSNYWVNWVRQSPEKGLEWVAQIRLKSENYPTHYAESVKGRFTVSRDDSKSSVYLQMNNLRAEDTGIYYCTGRGYSNYAWGQGTTLTVSS

SEQ ID NO: 201 - protein, 113 aa, Artificial Sequence
  802.3E6.2F9 VH
QVQLQQSGAELMKPGASVKISCKVTGHTFSSYWIEWVKQRPGHGLEWIGEILPGKGNTEYNEKFKGKATFTADTSSNTAYMQLSSLTSEDSAVYYCARNFDVWGAGTTVTVSS

SEQ ID NO: 202 - protein, 116 aa, Artificial Sequence
  802.3E6.2H9 VH
QVQKQQPGAELVMPGASVKLSCKASGYTFTSYWMHWVMQRPGQGLEWIGDIDPSDSYTNYNQQFKGKATLTVDISSSSAYMQLSSLTFEDSAVYYCARARVDFDYWGRGTTLTVSS

SEQ ID NO: 203 - protein, 113 aa, Artificial Sequence
  802.3G8.2A3 VH
EVQLQQSGAELVRPGASVKLSCTASGFNIKDDYMHWVKQRPEQGLEWIGWIDPENGDTAYASKFQGKATITADTSSNIAYLQLSSLTFEDTAVYYCTTGSTVWGQGTTLTVSS

SEQ ID NO: 204 - protein, 115 aa, Artificial Sequence
  802.3G8.2F7 VH
QIQLVQSGPELKKPGETVKISCKASGYTFTTYGMSWVKQAPGKGLKWMGWINTYSGVPTYTDDFKGRFAFSLETSASTAYLQINNLKNEDTATYFCALTGVLENWGQGTTLTVSS

SEQ ID NO: 205 - protein, 116 aa, Artificial Sequence
  802.3H4.2D11 VH
EVQLQQSGPELVKPGDSEKRSCKASGYTFTDYYMDWVKQSHGRSLEWIGYISPKNGGTSYNQKFKGKATLTIDKSSSTAYMELHSLTSEDSAVYYCASPFYDDYYWGQGTTLTVSS

SEQ ID NO: 206 - protein, 115 aa, Artificial Sequence
  802.3H4.2G3 VH
QAYLQHSGAELVRPGASVKMSCKASGYTFTSYNMHWVKQTPRQGLEWIGAIYPGNGYTSYNQKFMGKATLTVDKSSSTAYMQLTSLTSEDSAVYFCARGVGFDYWGQGTILTVSS

SEQ ID NO: 207 - protein, 118 aa, Artificial Sequence
  802.4B6.2E11 VH
QVQLQQPDTELVKPGASVKLSCRASGYTFTSYWINWVKQRPGQGLEWIGNIYPGTSSTNYNEKFKSKATLTVDPSSSTAYMQLSSLTSDDSAVYYCSRSSYRYTYYSWGQGTTLTVSS

SEQ ID NO: 208 - protein, 118 aa, Artificial Sequence
  802.4B6.2F6 VH
QVQLQQSGPELVKPGASVKLSCKASGYTFTSYDINWVKQRPGQGLEWIGWIYPRDGTTKYNEKFKGKATLTVDTSSSTAYMEIHSLTSEDSAVYFCAREDYGSSFHNWGQGTTLTVSS

SEQ ID NO: 209 - protein, 122 aa, Artificial Sequence
  802.4C12.3C5 VH
QVQLQQSGGELMKPGASVKLSCKATGYTFTGYWIEWVKQRPGHGLEWIGEILPGSGRTYYNEKFKGKATFTADTSSNTAYMQLSSLTTEDSAIYYCAREIYDGHYEAWYAYWGQGTLVTVSA

SEQ ID NO: 210 - protein, 118 aa, Artificial Sequence
  802.4H12.2A9 VH
EVQLVESGGGLVKPGGSLKLSCAASGFTFSSYTMSWVRQTPEKTLEWVATISDGGRYTYYPDNVKGRFTISRDNANNNLYLQMSHLKSEDTAMYYCARGWLLRNFDFWGQGTTLTVSS

SEQ ID NO: 211 - protein, 118 aa, Artificial Sequence
  802.4H12.2D2 VH
QVQLQQPGAELVKPGASVKLSCKASGYTFTSYWMHWVKQRPGRGLEWIGRIDPNSGGTKYNEKFKSKAILTVDKPSSTAYMQLSSLTSEDSAVYYCARLGYGNPFDYWGQGTTLTVSS

SEQ ID NO: 212 - protein, 113 aa, Artificial Sequence
  802.4H6.2D11 VH
  misc_feature (4)..(5): Xaa can be any naturally occurring amino acid
  misc_feature (7)..(7): Xaa can be any naturally occurring amino acid
  misc_feature (9)..(9): Xaa can be any naturally occurring amino acid
  misc_feature (32)..(32): Xaa can be any naturally occurring amino acid
EVQXXQXGXELVRPGASVKWSCTASGFKIKDXYMHWVKQRPEQGPEWIGWIDPENGDTEHASKFQGKATITVDTSSNTAYLQLSSLTSEDTAVYYCTTGSTVWGQGTTLTVSS

SEQ ID NO: 213 - protein, 120 aa, Artificial Sequence
  802.4H6.2F8 VH
EVQLQQSGTVLARPGASVKMSCKASGYTFTSYWMHWVKQRPGQGLEWIGAIYPGNSETSYNQNFKGKAELTAVTSASTAYMEISSLTNEDSAVYFCTRPLANWDGPFDYWGQGTTLTVSS

SEQ ID NO: 214 - protein, 118 aa, Artificial Sequence
  802.4H6.2G12 VH
EVKVEESGGGLVQPGGSMKFSCVASGFTFSNYWMNWVRQSPEKGLEWVAQIRLKSDNYATHYAESVKGRFTISRDDSKSSVYLQMNNLRAEDTGIYYCTGRGYSNYAWGQGTTLTVSS

SEQ ID NO: 215 - protein, 113 aa, Artificial Sequence
  802.5G6.2B11 VH
EVQLQQSGAELVRPGASVKLSCTASGFNIKDDYLHWVKQRPEQGLEWIGWIDPEKGDTECASKFQGKATITVDTSSNTAYLQLSSLTSEDTAVYFCTTGSTVWGQGTTLTVSS

SEQ ID NO: 216 - protein, 119 aa, Artificial Sequence
  802.5G6.2B8 VH
EVHLVESGGGLMKPGGSLKLSCAASGFTFSSYAMSWVRQTPEKRLEWVATISDGGSYTYYPDNVKGRFTISRDNAKNNLYLQMSRLKSEDTAMYYCSRAGSTVVNFGYWGQGTTLTVSS

SEQ ID NO: 217 - protein, 112 aa, Artificial Sequence
  802.1H3.2A4 VL
DVVMTQTPLSLPVSLGDQASISCRSSQSIVHSNGNTFLEWYLQKPGQSPNLLIYKVSNRFSGVPDRFSGSGSGTDFTLKISRVEAEDLGVYYCFQGSHVPLSFGAGTKLELK

SEQ ID NO: 218 - protein, 107 aa, Artificial Sequence
  802.2B7.2D9 VL
DIQMTQSPASLYAFVGETVTITCRASENIYSYLAWYHQKQGKSPQLLVYNSNSLAEGVPLRFSGSGSGTQFSLKINSLQPEDFGNYYCQHHYVTPLTFGGGTKLEMK

SEQ ID NO: 219 - protein, 112 aa, Artificial Sequence
  802.2B7.2F9 VL
DVVMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPLTFGGGTKLELK

SEQ ID NO: 220 - protein, 112 aa, Artificial Sequence
  802.2F11.2B6 VL
DVVMTQTPLSLSVTIGQPASISCKSSQSLLYSDGKTYLNWLQQRPGQSPKRLMYQVSKLDPGIPDRFSGSGSETDFTLKISRVEAEDLGVYYCLQGTYYPHTFGSGTKLEIK

SEQ ID NO: 221 - protein, 112 aa, Artificial Sequence
  802.2F4.2A3 VL
  misc_feature (7)..(7): Xaa can be any naturally occurring amino acid
  misc_feature (12)..(14): Xaa can be any naturally occurring amino acid
  misc_feature (33)..(33): Xaa can be any naturally occurring amino acid
  misc_feature (41)..(42): Xaa can be any naturally occurring amino acid
  misc_feature (44)..(44): Xaa can be any naturally occurring amino acid
  misc_feature (46)..(46): Xaa can be any naturally occurring amino acid
  misc_feature (52)..(52): Xaa can be any naturally occurring amino acid
  misc_feature (62)..(62): Xaa can be any naturally occurring amino acid
  misc_feature (68)..(68): Xaa can be any naturally occurring amino acid
  misc_feature (73)..(73): Xaa can be any naturally occurring amino acid
  misc_feature (83)..(83): Xaa can be any naturally occurring amino acid
  misc_feature (93)..(93): Xaa can be any naturally occurring amino acid
  misc_feature (105)..(105): Xaa can be any naturally occurring amino acid
NIMKRHXPSSQXXXSGEKVIGSCISSQSVFCRXIQKNHLAXXQXKXGQSKQXLIYWASRREXGVPDRXTGRGXGADFIPIISXVQEEDAADYXGQQYLSSWTFGXGTKLEIK

SEQ ID NO: 222 - protein, 112 aa, Artificial Sequence
  802.2F4.2C7 VL
DVVMTQTPLSLPVSLGDQASISCRSSQSLVHNDGNTYLHWFLQKPGQSPKLLIHKVSNRFSGVPDRFSGSGSGTDFTLKISRVEAEDLGVYFCSQSTHVPYTFGGGTKLEIK

SEQ ID NO: 223 - protein, 107 aa, Artificial Sequence
  802.3B10.2C10 VL
NIVMTQSPKSMSMLVGERVILNCKASENVDTYVSWYQQKPEQSPKLLIYGASNRYTGVPDRFTGSGSATDFTLTISSVQGEDLADYYCGQSHNYPYTFGSGTKLEIK

SEQ ID NO: 224 - protein, 111 aa, Artificial Sequence
  802.3D4.2D4 VL
  misc_feature (52)..(52): Xaa can be any naturally occurring amino acid
  misc_feature (61)..(61): Xaa can be any naturally occurring amino acid
DVVMTQTPLSLLVSLGDQASISRRFSQSIVHSNGNTYLEWYVQKPGQSPKLXIYKVSNRFXGVPDRFSGSGSGTDFTLKISRVEAEDLGVYYCFQGSHVPLTFGGGTKLEK

SEQ ID NO: 225 - protein, 112 aa, Artificial Sequence
  802.3D5.2G4 VL
DIVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHFPWTFGGGTKLEIK

SEQ ID NO: 226 - protein, 112 aa, Artificial Sequence
  802.3E6.2F9 VL
DVVMTQTPLSLPVSLGDQASISCRSSQSLVHRNGHTYLHWYLQKPGQSPKLLIYKVSNRFSGVPDRFSGSVSGTDFTLKISRVEGEDVGVYFCSQSTHVPYTFGGGTKLEIK

SEQ ID NO: 227 - protein, 108 aa, Artificial Sequence
  802.3E6.2H9 VL
EIVLTQSPALLAASPGDKVTITCSVSSSISSSTLHWYQQKSGTSPKPWIYGTSNLASGVPVRFSGSGSGTSYSLTISSMEAEDAATYYCQQWSGYPLTFGAGTKLELK

SEQ ID NO: 228 - protein, 74 aa, Artificial Sequence
  802.3G8.2A3 VL
  misc_feature (9)..(10): Xaa can be any naturally occurring amino acid
  misc_feature (13)..(14): Xaa can be any naturally occurring amino acid
  misc_feature (17)..(18): Xaa can be any naturally occurring amino acid
  misc_feature (21)..(21): Xaa can be any naturally occurring amino acid
  misc_feature (24)..(24): Xaa can be any naturally occurring amino acid
  misc_feature (31)..(33): Xaa can be any naturally occurring amino acid
  misc_feature (36)..(36): Xaa can be any naturally occurring amino acid
  misc_feature (48)..(49): Xaa can be any naturally occurring amino acid
YLNWLEQSXXGQXXRLXXYLXPTXNESLRDXXXAVXDSRLHPQHPSEXXRRMQLISASTTGSVHVRRGDQAGNK

SEQ ID NO: 229 - protein, 112 aa, Artificial Sequence
  802.3G8.2F7 VL
DVVMTQTPLSLPVSLGDQASISCRFTQSIVHSNGNTYLEWYLQKPGQSPNLLIYKVSNRFCGVPDRFSGSGSGTDFTLKISRVEPEDLGVYYCFQGSHVPFTFGTGTKLEIK

SEQ ID NO: 230 - protein, 112 aa, Artificial Sequence
  802.3H4.2D11 VL
DVVMTQTPLTLSVTIGQPASISCKSSQSLLHRNGKTYLNWLLQRPGQSPKLLIYLVSKLESGVPDRFSGSGSGTDFTLKISRVEAEDLGIYYCFQTTHFPHTFGSGTKLEIK

SEQ ID NO: 231 - protein, 112 aa, Artificial Sequence
  802.3H4.2G3 VL
DVVMTQTPLSLPVSLGDQASISCRSSQSIVHSNGNSYLDWYLQKPGQSPKLLIYKVSNRFSGVPDRFSGSGSGTDFTLKISRVEAEDLGVYYCFQGSHVPLTFGAGTKLELK

SEQ ID NO: 232 - protein, 112 aa, Artificial Sequence
  802.4B6.2E11 VL
DVVMTQTPLSLPVSLGDQASISCRSSQSIVQSNGITYLEWYLKKPGQSPNLLIYKVSNRLSGVPDRFSGSGSGTDFTLKISRVEADDLGVYYCFQGSHVPLTFGAGTKLELK

SEQ ID NO: 233 - protein, 107 aa, Artificial Sequence
  802.4B6.2F6 VL
NIVMTQSPKSMSMSVGERVTLSCKASEDVDTYVSWYQQKPNQSPKLLIYGASNRYTGVPDRFTGSGSATDFTLTISSVQAEDLADYYCGQSYSYPWTFGGGTKLEIK

SEQ ID NO: 234 - protein, 112 aa, Artificial Sequence
  802.4C12.3C5 VL
DVVMTQTPLSLSVTIGQPASISCKSSQSLLYSDGKTYLNWLQQRPGQSPKRLMYQVSKLDPGIPDRFSGSGSETDFTLEISRVEAEDLGVYYCLQGTYYPHTFGSGTKLEIK

SEQ ID NO: 235 - protein, 112 aa, Artificial Sequence
  802.4H12.2A9 VL
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 236 - protein, 107 aa, Artificial Sequence
  802.4H12.2D2 VL
DIQMTQSPASLSASVGETVTITCRASENIYSNLAWYQQKQGKSPQLLVYTATNLADGVPSRFSGSGSGTQFSLKINSLQPEDFGSYYCQHFYGSPRTFGAGTKLELK

SEQ ID NO: 237 - protein, 112 aa, Artificial Sequence
  802.4H6.2D11 VL
DVVMTQTPLTLSVTIGQPASISCKSSQSLLYSDGKTYLNWLLQSPGQSPKLLIYLVSKLESGVPDRFSGRGSGTDFTLKISRVEAEDLGVYYCVQGIHFPLTFGTGTKLEMK

SEQ ID NO: 238 - protein, 106 aa, Artificial Sequence
  802.4H6.2F8 VL
NIVMTQSPKSMSMSVGERVTLTCKASENVGTYVSWFQQKPEQSPKLLIYGASNRYTGVPDRFTGSGSATDFTLTISSVQAEDLADYYCGQSYTDLTFGAGTKLELK

SEQ ID NO: 239 - protein, 112 aa, Artificial Sequence
  802.4H6.2G12 VL
DVVMTQTPLSLPVSLGNQASISCRSSQNIVHTNGNTYLEWYLQKPGQSPKLLIYKVSNRFCGVPDRFSGSGSGTDFTLKISRVEAEDLGLYYCFQGSHIPWTFGGGTKLEIK

SEQ ID NO: 240 - protein, 112 aa, Artificial Sequence
  802.5G6.2B11 VL
DVVMTQTPLTLSVTIGQPASISCKSSQSLLYSDGKTYLNWLLQSPGQSPKLLIYLVSKLESGVPDRFSGSGSGTDFTLKISRVEAEDLGIYLCVQGPHFPLTFGGGTKLELK

SEQ ID NO: 241 - protein, 112 aa, Artificial Sequence
  802.5G6.2B8 VL
DIVMTQTPLSLPVSLGDQASISCRSSQNIAHSNGNTYLEWYLQKPGQSPQLLIYKVSNRFSGVPDRFSGSGSGTDFTLKISRVEAEDLGVYYCFQGSHVPLTFGAGTKLELK

SEQ ID NO: 242 - protein, 10 aa, Artificial Sequence
  CDR66-H
GFSLSTSGMA

SEQ ID NO: 243 - protein, 7 aa, Artificial Sequence
  CDR67-H
IWWDDDK

SEQ ID NO: 244 - protein, 10 aa, Artificial Sequence
  CDR68-H
ARRAYPYFDV

SEQ ID NO: 245 - protein, 9 aa, Artificial Sequence
  CDR69-H
ENSITSSYD

SEQ ID NO: 246 - protein, 6 aa, Artificial Sequence
  CDR43-L
ENIYSY

SEQ ID NO: 247 - protein, 3 aa, Artificial Sequence
  CDR44-L
NSN

SEQ ID NO: 248 - protein, 9 aa, Artificial Sequence
  CDR45-L
QHHYVTPLT

SEQ ID NO: 249 - protein, 3 aa, Artificial Sequence
  CDR46-L
WAS

SEQ ID NO: 250 - protein, 8 aa, Artificial Sequence
  CDR47-L
QQYLSSWT

SEQ ID NO: 251 - protein, 11 aa, Artificial Sequence
  CDR48-L
QSLVHNDGNTY

SEQ ID NO: 252 - protein, 9 aa, Artificial Sequence
  CDR49-L
SQSTLVPYT

SEQ ID NO: 253 - protein, 11 aa, Artificial Sequence
  CDR50-L
QSLVHRNGHTY

SEQ ID NO: 254 - protein, 9 aa, Artificial Sequence
  CDR51-L
SQSTHVPYT

(19) United States
(12) Patent Application Publication
(10) Pub. No.: US 2022/0281973 A1
(43) Pub. Date: Sep. 8, 2022

(54) ANTIBODIES AND BLOCKING THE INTERACTION OF VISTA AND ITS BINDING PARTNER

(71) Applicant: TrueBinding, Inc., Foster City, CA (US)

(72) Inventors: Dongxu Sun, Palo Alto, CA (US); Catherine A. Gordon, Seattle, WA (US); Yi Chai, Seattle, WA (US); Maja Zukic Bonacorsi, Santa Clara, CA (US); Samuel A.F. Williams, Burlingame, CA (US); Dalya Rivka Rosner, Redwood City, CA (US); Ksenya Shchors, San Mateo, CA (US)

(21) Appl. No.: 17/637,778

(22) PCT Filed: Aug. 27, 2020
(86) PCT No.: PCT/US2020/048235

§ 371 Date: Feb. 23, 2022

Related U.S. Application Data
(60) Provisional application No. 62/893,482, filed on Aug. 29, 2019.

(51) Int. Cl. C07K 16/28

(52) U.S. Cl. CPC C07K 16/2803; C07K 16/2827; C07K 2317/31; C07K 2317/92; C07K 2317/24; C07K 2317/565; C07K 2317/567; C07K 2317/55; C07K 2317/54; C07K 2317/569; C07K 2317/622; C07K 2317/626; C07K 2317/22

(57) ABSTRACT

Disclosed herein are antibodies that specifically bind to LRIG1 and methods of use thereof. In some embodiments, also described herein are methods of modulating immune system activity or promoting immune cell proliferation with an antibody that specifically binds to LRIG1.

### CROSS-REFERENCE TO RELATED APPLICATION

This application claims the benefit of priority of U.S. Provisional Patent Application No. 62/893,482, filed Aug. 29, 2019, which is hereby expressly incorporated by reference in its entirety.

### BIOLOGICAL SAMPLE DEPOSIT STATEMENT

In some embodiments, antibodies that bind to LRIG1 and block the binding of VISTA, which are deposited in the American Type Culture Collection (ATCC), in accordance with the Budapest Treaty, under the numbers ______, on ______. These antibodies are named: 802.4H6.2D11, 802.3D4.2D4, 802.2F4.2A3, and 802.3G8.2A3.

These deposits are made under the provisions of the Budapest Treaty on the International Recognition of the Deposit of Microorganisms for the Purposes of Patent Procedure and the Regulations thereunder (Budapest Treaty). This assures maintenance of the deposit for 30 years from date of deposit. The deposit will be made available by the ATCC under the terms of the Budapest Treaty, and subject to an agreement between Applicant and the ATCC, which assures permanent and unrestricted availability of the deposit to the public upon issuance of the pertinent U.S. patent or upon laying open to the public of any U.S. or foreign patent application, whichever comes first, and assures availability of the deposit to one determined by the U.S. Commissioner of Patents and Trademarks to be entitled thereto according to 35 U.S.C. § 122 and the Commissioner's Rules pursuant thereto (including 37 C.F.R. § 1.14). Availability of the deposited biological material is not to be construed as a license to practice the invention in contravention of the rights granted under the authority of any Government in accordance with its Patent Laws.

### REFERENCE TO SEQUENCE LISTING

The present application is being filed along with a Sequence Listing in electronic format. The Sequence Listing is provided as a file entitled SeqListingIMMUT014WO.TXT, which was created and last modified on Aug. 27, 2020, which is 129,585 bytes in size. The information in the electronic Sequence Listing is hereby incorporated by reference in its entirety.

### FIELD

Aspects of the present disclosure are directed to antibodies that specifically bind to LRIG1 and methods of use thereof. In some embodiments, also described herein are methods of modulating immune system activity or promoting immune cell proliferation with an antibody that specifically binds to LRIG1.

### BACKGROUND

LRIG1 (leucine-rich repeats and immunoglobulin-like domains protein 1) is a transmembrane protein that has tumor suppressor function and has also been implicated in proliferation and quiescence of normal cells. VISTA (V-domain Ig suppressor of T cell activation) is a transmembrane immune checkpoint protein that is expressed in a wide range of white blood cells and is one binding. VISTA activity is implicated in many immune-related diseases, including autoimmune and inflammatory disorders and cancer.

### SUMMARY OF THE DISCLOSURE

There is a lasting need for therapeutic interventions for immune diseases and cancer, including those regulated by LRIG1 and/or VISTA.

Disclosed herein, in some embodiments, are anti-LRIG1 antibodies and methods of using the same to modulate immune function, for example, inducing or inhibiting immune activation. Further disclosed herein, in some embodiments, are methods of using anti-LRIG1 antibodies to promote proliferation of immune cells (e.g. dendritic cells, B cells, cytotoxic T cells, regulatory T cells, and/or Natural Killer cells). In some embodiments, the modulation of immune function by the anti-LRIG1 antibodies disclosed herein comprise either activation or inhibition of immune function. In some embodiments, activation of immune function comprises upregulating activity of immune cells such as B cells and/or cytotoxic T cells. In some embodiments, inhibition of immune function comprises downregulating the activity of immune cells, or upregulating the proliferation and/or function of immunosuppressive cells such as regulatory T cells.

Disclosed herein, in some embodiments, is an antigen binding polypeptide, wherein the polypeptide exhibits specific binding to LRIG1 protein (SEQ ID NO: 2), and wherein the polypeptide binds to an epitope present on one or more regions of LRIG1 selected from a group consisting of any amino acid sequence from amino acid residues from position 1 to 564 or position 655 to 1093 from N terminus to C terminus of LRIG1 protein. In some embodiments, the polypeptide binds to an epitope present within an amino acid sequence from amino acid residues from position 674 to 714 from N terminus to C terminus of LRIG1 protein. In some embodiments, the polypeptide binds to an epitope present within an amino acid sequence from amino acid residues from position 704 to 744 from N terminus to C terminus of LRIG1 protein. In some embodiments, the polypeptide binds to an undetermined epitope of LRIG1 between position 1 to 564 or position 655 to 1093 from N terminus to C terminus. In some embodiments, the antibody comprises a full-length antibody or a fragment thereof. In some embodiments, the antibody comprises a bispecific antibody or a binding fragment thereof. In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the polypeptide comprises at least one complementarity-defining region (CDR) selected from SEQ ID NOs: 84-145, 149-187, or 242-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises 3 heavy chain CDRs and 3 light chain CDRs according to FIG. 9. In some embodiments, the antibody comprises an IgG framework. In some embodiments, the antibody comprises an IgG1, IgG2, or IgG4 framework. In some embodiments, the antibody comprises a kD of less than 1 nM, 1.2 nM, 2 nM, 5 nM, 10 nM, 13.5 nM, 15 nM, 20 nM, 25 nM, or 30 nM. In some embodiments, the antibody comprises a humanized antibody. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antibody or antigen binding polypeptide comprises a VH and VL according to Table 5. In some embodiments, the antibody is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5 G6.2B 8.

Disclosed herein, in some embodiments, is an antigen binding polypeptide, wherein the polypeptide disrupts LRIG1-VISTA interaction, and wherein the polypeptide binds to an epitope of LRIG1 in a region from SEQ ID NO: 69 to 75. In some embodiments, the polypeptide binds to an epitope present within the region of LRIG1 defined by Peptide 65 (SEQ. ID No. 69). In some embodiments, the polypeptide binds to an epitope present within the region of LRIG1 defined by Peptide 66 (SEQ. ID No. 70). In some embodiments, the polypeptide binds to an epitope present within the region of LRIG1 defined by Peptide 67 (SEQ. ID No. 71). In some embodiments, the polypeptide binds to an epitope present within the region of LRIG1 defined by Peptide 68 (SEQ. ID No. 72). In some embodiments, the polypeptide binds to an epitope present within the region of LRIG1 defined by Peptide 69 (SEQ. ID No. 73). In some embodiments, the polypeptide binds to an epitope present within the region of LRIG1 defined by Peptide 70 (SEQ. ID No. 74). In some embodiments, the polypeptide binds to an epitope present within the region of LRIG1 defined by Peptide 71 (SEQ. ID No. 75). In some embodiments, the antibody comprises a full-length antibody or a fragment thereof. In some embodiments, the antibody comprises a bispecific antibody or a binding fragment thereof. In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the polypeptide comprises at least one complementarity-defining region selected from SEQ ID NOs: 84-145, 149-187, or 242-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or antigen binding polypeptide comprises 3 heavy chain CDRs and 3 light chain CDRs according to FIG. 9. In some embodiments, the antibody comprises an IgG framework. In some embodiments, the antibody comprises an IgG1, IgG2, or IgG4 framework. In some embodiments, the antibody comprises a kD of less than 1 nM, 1.2 nM, 2 nM, 5 nM, 10 nM, 13.5 nM, 15 nM, 20 nM, 25 nM, or 30 nM. In some embodiments, the antibody comprises a humanized antibody. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antibody or antigen binding polypeptide comprises a VH and VL according to Table 5. In some embodiments, the antibody is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5 G6.2B 8.

Disclosed herein, in some embodiments, is an antigen binding polypeptide, wherein the polypeptide exhibits specific binding to LRIG1 protein, wherein the binding of the polypeptide to LRIG1 reduces an interaction between LRIG1-VISTA to less than 21% of interaction between LRIG1-VISTA without the antigen binding polypeptide. In some embodiments, the binding of the polypeptide to LRIG1 reduces an interaction between LRIG1-VISTA to less than 20%, less than 15%, less than 10%, less than 5%, or less than 1% of the interaction between LRIG1-VISTA without the antigen binding polypeptide. In some embodiments, the polypeptide binds to an epitope of LRIG1 in a region from SEQ ID NO: 69 to 75. In some embodiments, the antibody comprises a full-length antibody or a fragment thereof. In some embodiments, the antibody comprises a bispecific antibody or a binding fragment thereof. In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the polypeptide comprises at least one complementarity-defining region selected from SEQ ID NOs: 84-145, 149-187, or 242-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or antigen binding polypeptide comprises 3 heavy chain CDRs and 3 light chain CDRs according to FIG. 9. In some embodiments, the antibody comprises an IgG framework. In some embodiments, the antibody comprises an IgG1, IgG2, or IgG4 framework. In some embodiments, the antibody comprises a kD of less than 1 nM, 1.2 nM, 2 nM, 5 nM, 10 nM, 13.5 nM, 15 nM, 20 nM, 25 nM, or 30 nM. In some embodiments, the antibody comprises a humanized antibody. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antibody or antigen binding polypeptide comprises a VH and VL according to Table 5. In some embodiments, the antibody is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8.

Disclosed herein, in some embodiments, is an antigen binding polypeptide, wherein the polypeptide comprises at least one complementarity-defining region selected from SEQ ID NOs: 84-145, 149-187, or 242-254, and wherein the polypeptide is capable of binding an epitope present in one or more regions of LRIG1 protein. In some embodiments, the antibody comprises a full-length antibody or a fragment thereof. In some embodiments, the antibody comprises a bispecific antibody or a binding fragment thereof. In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the polypeptide comprises more than one complementarity-defining region selected from SEQ ID NOs: 84-145, 149-187, or 242-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or antigen binding polypeptide comprises 3 heavy chain CDRs and 3 light chain CDRs according to FIG. 9. In some embodiments, the antibody comprises an IgG framework. In some embodiments, the antibody comprises an IgG1, IgG2, or IgG4 framework. In some embodiments, the antibody comprises a kD of less than 1 nM, 1.2 nM, 2 nM, 5 nM, 10 nM, 13.5 nM, 15 nM, 20 nM, 25 nM, or 30 nM. In some embodiments, the antibody comprises a humanized antibody. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antibody or antigen binding polypeptide comprises a VH and VL according to Table 5. In some embodiments, the antibody is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8.

Disclosed herein, in some embodiments, is an antigen binding polypeptide, wherein the polypeptide exhibits specific binding to LRIG1 protein such that upon binding said polypeptide reduces an interaction between LRIG1-VISTA by (i) at least 79%, wherein said antibody is not IMT300 or (ii) a greater degree than IMT300.

Disclosed herein, in some embodiments, is a complex comprising any of the above-described polypeptides, wherein the complex comprises the polypeptide bound to LRIG1 protein.

Disclosed herein, in some embodiments, is a method of disrupting an interaction between VISTA and LRIG1 (to, for example, modulate VISTA biology), comprising contacting a plurality of cells comprising a LRIG1-expressing cell, a VISTA-expressing cell, or a combination thereof with any of the above-described antigen binding polypeptides. In some embodiments, the LRIG1-VISTA interaction is reduced to less than 21%, less than 20%, less than 19%, less than 17%, less than 10%, less than 5%, or less than 1%. In some embodiments, the interaction occurs at one or more residues of LRIG1 selected from region 245-260, wherein the residue positions correspond to positions 245-260 of SEQ ID NO: 2. In some embodiments, the interaction occurs at one or more residues of VISTA selected from region 78-90 or 68-92, wherein the residue positions correspond to positions 78-90 or 68-92 of SEQ ID NO: 4. In some embodiments, the antibody binds to at least one amino acid residue within any peptide from SEQ ID NO. 69 to 75. In some embodiments, the antibody comprises a kD of less than 1 nM, 1.2 nM, 2 nM, 5 nM, 10 nM, 13.5 nM, 15 nM, 20 nM, 25 nM, or 30 nM. In some embodiments, the antibody comprises a humanized antibody. In some embodiments, the antibody comprises a full-length antibody or a binding fragment thereof. In some embodiments, the antibody comprises a bispecific antibody or a binding fragment thereof. In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody is a humanized antibody comprising at least one complementarity-determining regions (CDRs) from SEQ ID NOs: 84-145, 149-187, or 242-254. In some embodiments, the humanized antibody comprises a heavy chain variable region (VH) comprising one, two, or three CDR sequences selected from SEQ ID NOs: 84 to 145 or 242-245. In some embodiments, the humanized antibody comprises a light chain variable region (VL) comprising one, two, or three CDR sequences selected from SEQ ID NOs: 149 to 187 or 246-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or antigen binding polypeptide comprises 3 heavy chain CDRs and 3 light chain CDRs according to FIG. 9. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antibody or antigen binding polypeptide comprises a VH and VL according to Table 5. In some embodiments, the antibody is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8. In some embodiments, the antibody comprises an IgG framework. In some embodiments, the antibody comprises an IgG1, IgG2, or IgG4 framework.

In some embodiments, the constructs can be used in the treatment of autoimmune diseases.

Embodiments of the present invention provided herein are described by way of the following numbered alternatives:

1. An antigen binding polypeptide, wherein the polypeptide exhibits specific binding to LRIG1 protein (SEQ ID NO: 2), and wherein the polypeptide binds to an epitope present on one or more regions of LRIG1 selected from a group consisting of any amino acid sequence from amino acid residues from position 1 to 564 or position 655 to 1093 from N terminus to C terminus of LRIG1 protein.

2. The antigen binding polypeptide of alternative 1, wherein the polypeptide binds to an epitope present within an amino acid sequence from amino acid residues from position 674 to 714 from N terminus to C terminus of LRIG1 protein.

3. The antigen binding polypeptide of alternative 1 or 2, wherein the polypeptide binds to an epitope present within an amino acid sequence from amino acid residues from position 704 to 744 from N terminus to C terminus of LRIG1 protein.

4. The antigen binding polypeptide of any one of the alternatives 1-3, wherein the polypeptide binds to an undetermined epitope of LRIG1 between position 1 to 564 or position 655 to 1093 from N terminus to C terminus.

5. The antigen binding polypeptide of any one of the alternatives 1-4, wherein the antigen binding polypeptide comprises a full-length antibody or a fragment thereof.

6. The antigen binding polypeptide of any one of the alternatives 1-5, wherein the antigen binding polypeptide comprises a bispecific antibody or a binding fragment thereof.

7. The antigen binding polypeptide of any one of the alternatives 1-6, wherein the antigen binding polypeptide comprises a monovalent Fab′, a divalent Fab2, a single-chain variable fragment (scFv), a diabody, a minibody, a nanobody, a single-domain antibody (sdAb), or a camelid antibody or binding fragment thereof.

8. The antigen binding polypeptide of any one of the alternatives 1-7, wherein the polypeptide comprises at least one complementarity-defining region selected from SEQ ID NOs: 84-145, 149-187, or 242-254.

9. The antigen binding polypeptide of any one of the alternatives 1-8, wherein the antigen binding polypeptide comprises an IgG framework.

10. The antigen binding polypeptide of any one of the alternatives 1-9, wherein the antigen binding polypeptide comprises an IgG1, IgG2, or IgG4 framework.

11. The antigen binding polypeptide of any one of the alternatives 1-10, wherein the antigen binding polypeptide comprises a k_{D} of less than 1 nM, 1.2 nM, 2 nM, 5 nM, 10 nM, 13.5 nM, 15 nM, 20 nM, 25 nM, or 30 nM.

12. The antigen binding polypeptide of any one of the alternatives 1-11, wherein the antigen binding polypeptide comprises a humanized antibody.

13. The antigen binding polypeptide of any one of the alternatives 1-12, wherein the antigen binding polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8.

14. An antigen binding polypeptide, wherein the polypeptide disrupts LRIG1-VISTA interaction, and wherein the polypeptide binds to an epitope of LRIG1 in a region from SEQ ID NOs: 69 to 75.

15. The antigen binding polypeptide of alternative 14, wherein the polypeptide binds to an epitope present within the region of LRIG1 defined by Peptide 65 (SEQ ID NO: 69).

16. The antigen binding polypeptide of alternative 14 or 15, wherein the polypeptide binds to an epitope present within the region of LRIG1 defined by Peptide 66 (SEQ ID NO: 70).

17. The antigen binding polypeptide of any one of the alternatives 14-6, wherein the polypeptide binds to an epitope present within the region of LRIG1 defined by Peptide 67 (SEQ ID NO: 71).

18. The antigen binding polypeptide of any one of the alternatives 14-17, wherein the polypeptide binds to an epitope present within the region of LRIG1 defined by Peptide 68 (SEQ ID NO: 72).

19. The antigen binding polypeptide of any one of the alternatives 14-18, wherein the polypeptide binds to an epitope present within the region of LRIG1 defined by Peptide 69 (SEQ ID NO: 73).

20. The antigen binding polypeptide of any one of the alternatives 14-19, wherein the polypeptide binds to an epitope present within the region of LRIG1 defined by Peptide 70 (SEQ ID NO: 74).

21. The antigen binding polypeptide of any one of the alternatives 14-20, wherein the polypeptide binds to an epitope present within the region of LRIG1 defined by Peptide 71 (SEQ ID NO: 75).

22. The antigen binding polypeptide of any one of the alternatives 14-21, wherein the antigen binding polypeptide comprises a full-length antibody or a fragment thereof.

23. The antigen binding polypeptide of any one of the alternatives 14-22, wherein the antigen binding polypeptide comprises a bispecific antibody or a binding fragment thereof.

24. The antigen binding polypeptide of any one of the alternatives 14-23, wherein the antigen binding polypeptide comprises a monovalent Fab′, a divalent Fab2, a single-chain variable fragment (scFv), a diabody, a minibody, a nanobody, a single-domain antibody (sdAb), or a camelid antibody or binding fragment thereof.

25. The antigen binding polypeptide of any one of the alternatives 14-24, wherein the polypeptide comprises at least one complementarity-defining region selected from SEQ ID NOs: 84-145, 149-187, or 242-254.

26. The antigen binding polypeptide of any one of the alternatives 14-25, wherein the antigen binding polypeptide comprises an IgG framework.

27. The antigen binding polypeptide of any one of the alternatives 14-26, wherein the antigen binding polypeptide comprises an IgG1, IgG2, or IgG4 framework.

28. The antigen binding polypeptide of any one of the alternatives 14-27, wherein the antigen binding polypeptide comprises a k_{D} of less than 1 nM, 1.2 nM, 2 nM, 5 nM, 10 nM, 13.5 nM, 15 nM, 20 nM, 25 nM, or 30 nM.

29. The antigen binding polypeptide of any one of the alternatives 14-28, wherein the antigen binding polypeptide comprises a humanized antibody.

30. The antigen binding polypeptide of any one of the alternatives 14-29, wherein the antigen binding polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8.

31. An antigen binding polypeptide, wherein the polypeptide exhibits specific binding to LRIG1 protein, wherein the binding of the polypeptide to LRIG1 reduces an interaction between LRIG1-VISTA to less than 21% of interaction between LRIG1-VISTA without the antigen binding polypeptide.

32. The antigen binding polypeptide of alternative 31, wherein the binding of the polypeptide to LRIG1 reduces an interaction between LRIG1-VISTA to less than 20%, less than 15%, less than 10%, less than 5%, or less than 1% of the interaction between LRIG1-VISTA without the antigen binding polypeptide.

33. The antigen binding polypeptide of alternative 31 or 32, wherein the polypeptide binds to an epitope of LRIG1 in a region from SEQ ID NOs: 69 to 75.

34. The antigen binding polypeptide of any one of the alternatives 31-33, wherein the antigen binding polypeptide comprises a full-length antibody or a fragment thereof.

35. The antigen binding polypeptide of any one of the alternatives 31-34, wherein the antigen binding polypeptide comprises a bispecific antibody or a binding fragment thereof.

36. The antigen binding polypeptide of any one of the alternatives 31-35, wherein the antigen binding polypeptide comprises a monovalent Fab′, a divalent Fab2, a single-chain variable fragment (scFv), a diabody, a minibody, a nanobody, a single-domain antibody (sdAb), or a camelid antibody or binding fragment thereof.

37. The antigen binding polypeptide of any one of the alternatives 31-36, wherein the polypeptide comprises at least one complementarity-defining region selected from SEQ ID NOs: 84-145, 149-187, or 242-254.

38. The antigen binding polypeptide of any one of the alternatives 31-37, wherein the antigen binding polypeptide comprises an IgG framework.

39. The antigen binding polypeptide of any one of the alternatives 31-38, wherein the antigen binding polypeptide comprises an IgG1, IgG2, or IgG4 framework.

40. The antigen binding polypeptide of any one of the alternatives 31-39, wherein the antigen binding polypeptide comprises a k_{D} of less than 1 nM, 1.2 nM, 2 nM, 5 nM, 10 nM, 13.5 nM, 15 nM, 20 nM, 25 nM, or 30 nM.

41. The antigen binding polypeptide of any one of the alternatives 31-40, wherein the antigen binding polypeptide comprises a humanized antibody.

42. The antigen binding polypeptide of any one of the alternatives 31-41, wherein the antigen binding polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8.

43. An antigen binding polypeptide, wherein the polypeptide comprises at least one complementarity-defining region selected from SEQ ID NOs: 84-145, 149-187, or 242-254, and wherein the polypeptide is capable of binding an epitope present in one or more regions of LRIG1 protein.

44. The antigen binding polypeptide of alternative 43, wherein the antigen binding polypeptide comprises a full-length antibody or a fragment thereof.

45. The antigen binding polypeptide of alternative 43 or 44, wherein the antigen binding polypeptide comprises a bispecific antibody or a binding fragment thereof.

46. The antigen binding polypeptide of any one of the alternatives 43-45, wherein the antigen binding polypeptide comprises a monovalent Fab′, a divalent Fab2, a single-chain variable fragment (scFv), a diabody, a minibody, a nanobody, a single-domain antibody (sdAb), or a camelid antibody or binding fragment thereof.

47. The antigen binding polypeptide of any one of the alternatives 43-46, wherein the polypeptide comprises more than one complementarity-defining region selected from SEQ ID NOs: 84-145, 149-187, or 242-254.

48. The antigen binding polypeptide of any one of the alternatives 43-47, wherein the antigen binding polypeptide comprises an IgG framework.

49. The antigen binding polypeptide of any one of the alternatives 43-48, wherein the antigen binding polypeptide comprises an IgG1, IgG2, or IgG4 framework.

50. The antigen binding polypeptide of any one of the alternatives 43-49, wherein the antigen binding polypeptide comprises a k_{D} of less than 1 nM, 1.2 nM, 2 nM, 5 nM, 10 nM, 13.5 nM, 15 nM, 20 nM, 25 nM, or 30 nM.

51. The antigen binding polypeptide of any one of the alternatives 43-50, wherein the antigen binding polypeptide comprises a humanized antibody.

52. The antigen binding polypeptide of any one of the alternatives 43-51, wherein the antigen binding polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8.

53. An antigen binding polypeptide, wherein the polypeptide exhibits specific binding to LRIG1 protein such that upon binding said polypeptide reduces an interaction between LRIG1-VISTA by (i) at least 79%, wherein said antibody is not IMT300 or (ii) a greater degree than IMT300.

54. A complex comprising the antigen binding polypeptide of any of alternatives 1-53, wherein the complex comprises the polypeptide bound to LRIG1 protein.

55. A method of disrupting an interaction between VISTA and LRIG1, comprising:

contacting a plurality of cells comprising a LRIG1-expressing cell, a VISTA-expressing cell, or a combination thereof with the antigen binding polypeptide from any one of alternatives 1-54.

56. The method of alternative 55, wherein the LRIG1-VISTA interaction is reduced to less than 21%, less than 20%, less than 19%, less than 17%, less than 10%, less than 5%, or less than 1%.

57. The method of alternative 55 or 56, wherein the interaction occurs at one or more residues of LRIG1 selected from region 245-260, wherein the residue positions correspond to positions 245-260 of SEQ ID NO: 2.

58. The method of any one of the alternatives 55-57, wherein the interaction occurs at one or more residues of VISTA selected from region 78-90 or 68-92, wherein the residue positions correspond to positions 78-90 or 68-92 of SEQ ID NO: 4.

59. The method of any one of the alternatives 55-58, wherein the antigen binding polypeptide binds to at least one amino acid residue within any peptide from SEQ ID NO. 69 to 75.

60. The method of any one of the alternatives 55-59, wherein the antigen binding polypeptide comprises a k_{D} of less than 1 nM, 1.2 nM, 2 nM, 5 nM, 10 nM, 13.5 nM, 15 nM, 20 nM, 25 nM, or 30 nM.

61. The method of any one of the alternatives 55-60, wherein the antigen binding polypeptide comprises a humanized antibody.

62. The method of any one of the alternatives 55-61, wherein the antigen binding polypeptide comprises a full-length antibody or a binding fragment thereof.

63. The method of any one of the alternatives 55-62, wherein the antigen binding polypeptide comprises a bispecific antibody or a binding fragment thereof.

64. The method of any one of the alternatives 55-63, wherein the antigen binding polypeptide comprises a monovalent Fab′, a divalent Fab2, a single-chain variable fragment (scFv), a diabody, a minibody, a nanobody, a single-domain antibody (sdAb), or a camelid antibody or binding fragment thereof.

65. The method of any one of the alternatives 55-64, wherein the antigen binding polypeptide is a humanized antibody comprising at least one complementarity-determining regions (CDRs) from SEQ ID NOs: 84-145, 149-187, or 242-254.

66. The method of any one of the alternatives 55-65, wherein the humanized antibody comprises a heavy chain variable region (VH) comprising any three sequences of SEQ ID NOs: 84 to 145 or 242-245.

67. The method of any one of the alternatives 55-66, wherein the humanized antibody comprises a light chain variable region (VL) comprising any three sequences of SEQ ID NOs: 149 to 187 or 246-254.

68. The method of any one of the alternatives 55-67, wherein the antigen binding polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5 G6.2B 11, or 802.5 G6.2B 8.

69. The method of any one of the alternatives 55-68, wherein the antigen binding polypeptide comprises an IgG framework.

70. The method of any one of the alternatives 59-69, wherein the antigen binding polypeptide comprises an IgG1, IgG2, or IgG4 framework.

71. A method of modulating an immune response in a subject, comprising: administering to the subject an antigen binding polypeptide that specifically binds to an LRIG1 protein (SEQ ID NO: 2).

72. The method of alternative 71, wherein the antigen binding peptide disrupts an interaction between LRIG1 and VISTA.

73. The method of alternative 71 or 72, wherein modulating the immune response comprises enhancing the level of T-cell-mediated and/or B-cell-mediated immune response in the subject.

74. The method of any one of the alternatives 71-73, wherein modulating the immune response comprises reducing the level of T-cell-mediated and/or B-cell-mediated immune response, or increasing the immunosuppressive function of regulatory T cells in the subject.

75. The method of any one of alternatives 71-74, wherein the subject comprises a cancer, and modulating the immune response ameliorates, treats, or reduces symptoms of the cancer.

76. The method of any one of the alternatives 71-75, wherein the cancer is breast cancer, colorectal cancer, kidney cancer, liver cancer, lung cancer, brain cancer, pancreatic cancer, bladder cancer, or stomach cancer, a hematologic malignancy, or any combination thereof.

77. The method of any one of alternatives 71-76, wherein the subject comprises an immune-related disorder, and modulating the immune response ameliorates, treats, or reduces symptoms of the immune-related disorder.

78. The method of any one of the alternatives 71-77, wherein the immune-related disorder is an autoimmune disease, an inflammatory disease, wound healing, fibrosis, liver fibrosis, kidney fibrosis, cardiac fibrosis, pulmonary fibrosis, non-alcoholic fatty liver disease, non-alcoholic steatohepatitis, sepsis, atopic dermatitis, psoriasis, systemic lupus erythematosus, inflammatory bowel syndrome, arthritis, or any combination thereof.

79. The method of any one of alternatives 71-78, further comprising an additional therapeutic agent to the subject.

80. The method of any one of the alternatives 71-79, wherein the additional therapeutic agent comprises an immunotherapeutic agent, an immune checkpoint modulator, a chemotherapeutic agent, targeted therapeutic agent, hormonal therapeutic agent, or a stem cell-based therapeutic agent.

81. The method of any one of alternatives 71-80, wherein the antigen binding polypeptide is administered parenterally.

82. The method of any one of alternatives 71-81, wherein the antigen binding polypeptide is the antigen binding polypeptide of any one of alternatives 1-53.

83. The method of any one of alternatives 71-82, wherein the antigen binding polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B 11, or 802.5 G6.2B 8

84. The method of any one of alternatives 71-83, wherein the subject is a mammal.

85. The method of any one of alternatives 71-84, wherein the subject is a human.

86. The antigen binding polypeptide of any one of alternatives 1-53 for use in the treatment of a cancer in a subject in need thereof.

87. The antigen binding polypeptide for use according to alternative 86, wherein the cancer is breast cancer, colorectal cancer, kidney cancer, liver cancer, lung cancer, brain cancer, pancreatic cancer, bladder cancer, or stomach cancer, or a hematological malignancy, or any combination thereof.

88. The antigen binding polypeptide of any one of alternatives 1-53 for use in the treatment of an immune-related disorder.

89. The antigen binding polypeptide for use according to alternative 88, wherein the immune-related disorder is an autoimmune disease, an inflammatory disease, wound healing, fibrosis, liver fibrosis, kidney fibrosis, cardiac fibrosis, pulmonary fibrosis, non-alcoholic fatty liver disease, non-alcoholic steatohepatitis, sepsis, atopic dermatitis, psoriasis, or any combination thereof.

90. The antigen binding polypeptide for use according to any one of alternatives 86-89, wherein the subject is a mammal.

91. The antigen binding polypeptide for use according to any one of alternatives 86-90, wherein the subject is a human.

92. A pharmaceutical composition comprising the antigen binding polypeptide of any one of alternatives 1-53 and at least one pharmaceutically acceptable carrier, excipient, diluent, or adjuvant.

93. The antigen binding polypeptide of any one of alternatives 1-53 or 86-92, the complex of alternative 54, the method of any one of alternatives 55-85, or the pharmaceutical composition of alternative 92, wherein the antigen binding polypeptide comprises at least one heavy chain CDR1 selected from the group consisting of SEQ ID NOs: 84, 87, 90, 93, 96, 99, 102, 105, 108, 111, 114, 117, 120, 123, 126, 129, 134, 138, 143, and 242.

94. The antigen binding polypeptide, complex, method, or pharmaceutical composition of alternative 93, wherein the antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243.

95. The antigen binding polypeptide, complex, method, or pharmaceutical composition of alternative 93 or 94, wherein the antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244.

96. The antigen binding polypeptide, complex, method, or pharmaceutical composition of any one of alternatives 93-95, wherein the antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253.

97. The antigen binding polypeptide, complex, method, or pharmaceutical composition of any one of alternatives 93-96, wherein the antigen binding polypeptide comprises at least one light chain CDR2 selected from the group consisting of SEQ ID NOs: 150, 155, 158, 160, 164, 169, 176, 179, 185, 247, and 249.

98. The antigen binding polypeptide, complex, method, or pharmaceutical composition of any one of alternatives 93-97, wherein the antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252.

99. The antigen binding polypeptide, complex, method, or pharmaceutical composition of any one of alternatives 93-98, wherein the antigen binding polypeptide comprises 3 heavy chain CDRs selected from the group consisting of SEQ ID NOs: 81-145 and 242-245, and 3 light chain CDRs selected from the group consisting of SEQ ID NOs: 146-187 and 246-254.

100. The antigen binding polypeptide, complex, method, or pharmaceutical composition of any one of alternatives 93-99, wherein the antigen binding polypeptide comprises 3 heavy chain CDRs and 3 light chain CDRs according to FIG. 9.

101. The antigen binding polypeptide, complex, method, or pharmaceutical composition of any one of alternatives 93-100, wherein the antigen binding polypeptide comprises a VH domain having a sequence with at least 80%, 85%, 90%, 95%, 96%, 97%, 98%, 99%, or 100% homology to SEQ ID NOs: 192-216.

102. The antigen binding polypeptide, complex, method, or pharmaceutical composition of any one of alternatives 93-101, wherein the antigen binding polypeptide comprises a VL domain having a sequence with at least 80%, 85%, 90%, 95%, 96%, 97%, 98%, 99%, or 100% homology to SEQ ID NOs: 217-241.

103. The antigen binding polypeptide, complex, method, or pharmaceutical composition of any one of alternatives 93-102, wherein the antigen binding polypeptide comprises a VH and VL according to Table 5.

104. A method of modulating activity of an immune system, comprising: contacting a plurality of cells comprising a LRIG1-expressing cell, a VISTA-expressing cell, or a combination thereof with the antigen binding polypeptide from any one of alternatives 1-54.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIG. 1 depicts blocking data for the interaction of LRIG1 protein with VISTA protein in the presence of anti-LRIG1 antibodies.

FIG. 2 depicts the extent of blockage of the interaction between LRIG1 protein and VISTA protein based upon the epitope region to which an anti-LRIG1 protein binds.

FIG. 3 depicts results from competitive binding assays of anti-LRIG1 proteins.

FIG. 4 depicts LRIG1-VISTA blocking data and complementarity-determining region (CDR) data for anti-LRIG1 antibodies.

FIG. 5A-5C depict MALDI-MS identification of LRIG1 and VISTA regions mediating the interaction between LRIG1 and VISTA. FIG. 5A and FIG. 5C illustrate the interaction site and residues within the site. FIG. 5B illustrates a depiction of crystal structure of LRIG1 highlighting the region mediating the interaction.

FIG. 6 depicts nucleic acid and protein sequences of LRIG1 and VISTA.

FIG. 7 depicts peptide sequences of LRIG1 used to generate antibodies.

FIG. 8 depicts complementarity-determining regions (CDRs) for anti-LRIG1 antibodies disclosed herein.

FIG. 9 depicts heavy and light CDR sequences for anti-LRIG1 antibodies disclosed herein.

FIG. 10 depicts exemplary heavy chain variable (VH) and light chain variable (VL) domains.

FIG. 11 depicts VH and VL combinations for additional anti-LRIG1 antibody embodiments.

FIG. 12 depicts additional exemplary heavy chain variable (VH) domains. The “x” denotes a residue that is undefined. In some embodiments, the antibody can be one that has any amino acid at that position. In some embodiments, the antibody can be one that includes all or at least 90% of the remaining, non-x, sequences in the figure (e.g., 91, 92, 93, 94, 95, 96, 97, 98, 99, or 100% of the explicitly defined residues)

FIG. 13 depicts additional exemplary light chain variable (VL) domains. The “x” denotes a residue that is undefined. In some embodiments, the antibody can be one that has any amino acid at that position. In some embodiments, the antibody can be one that includes all or at least 90% of the remaining, non-x, sequences in the figure (e.g., 91, 92, 93, 94, 95, 96, 97, 98, 99, or 100% of the explicitly defined residues)

### DETAILED DESCRIPTION OF THE DISCLOSURE

Provided herein are embodiments that related to anti-LRIG1 antigen binding polypeptides (e.g. antibodies) and their use in methods for disrupting an interaction between LRIG1 and an interacting protein, such as VISTA. In some embodiments, the interaction between LRIG1 and the interacting protein occurs between a cell expressing LRIG1 and a cell expressing the interacting protein. In some embodiments, the cell expressing LRIG1 and/or the cell expressing VISTA is an immune cell. In some embodiments, the cell expressing LRIG1 is a non-immune cell, and the cell expressing the interacting protein is an immune cell. In some embodiments, disrupting the interaction between LRIG1 and the interacting protein modulates immune function of immune cells, for example, the cell expressing the interacting protein. Also disclosed herein are methods for modulating an immune response in a subject. In some embodiments, the methods comprise administering an anti-LRIG antigen binding polypeptide to disrupt an interaction between LRIG1 and an interacting protein, such as VISTA. In some embodiments, modulating the immune response in the subject with the anti-LRIG antigen binding polypeptide may be used to ameliorate, treat, or reduce symptoms of a cancer or an immune-related disorder.

Tumors are often associated with an immune infiltrate as part of the reactive stroma that is enriched for macrophages. Tumor-associated macrophages (TAMs) play an important role in facilitating tumor growth by promoting neovascularization and matrix degradation. When associated with tumors, macrophages demonstrate functional polarization towards one of two phenotypically different subsets of macrophages: M1 macrophages or M2 macrophages. M1 macrophages are known to produce pro-inflammatory cytokines and play an active role in cell destruction, while M2 macrophages primarily scavenge debris and promote angiogenesis and wound repair. Consequently, many tumors with a high number of TAMs have an increased tumor growth rate, local proliferation, and distant metastasis. The M2 macrophage population is phenotypically similar to the TAM population that promotes tumor growth and development. In addition to expressing VISTA, M2 macrophages, in some cases, also express one or more cell surface markers selected from the group consisting of CD206, IL-4r, IL-1ra, decoy IL-1rll, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1.

VISTA (V-domain Ig suppressor of T cell activation) is expressed in high levels in myeloid cells, which include immature myeloid cells, monocytes, macrophages, neutrophils, basophils, eosinophils, erythrocytes, dendritic cells, megakaryocytes and platelets. VISTA levels are heightened within the tumor microenvironment. Furthermore, as indicated by the involvement of immune-regulating TAMs and other immune cells in the tumor microenvironment, which are only a subset of cells expressing this protein, VISTA is also involved in other immune-related disorders which include, but are not limited to autoimmune diseases, inflammatory diseases, psoriasis, systemic lupus erythematosus, inflammatory bowel syndrome, arthritis, or wound healing. Thus, in some embodiments, the compositions (antibodies, etc.) provided herein can be employed to address these disorders as well.

LRIG1 (Leucine-rich repeats and immunoglobulin-like domains protein 1) is a transmembrane protein that has been shown to interact with receptor tyrosine kinases of the EGFR-family, MET and RET. In some instances, LRIG1 has found to be a tumor suppressor and negative regulator of receptor tyrosine kinases. Abnormal LRIG1 function contributes to many aspects of cancer development, including proliferation, epithelial-mesenchymal transition, invasion, and metastatic spread of malignant cells.

In some embodiments, disclosed herein are anti-LRIG1 antibodies that, for example, interfere with the interaction between VISTA and LRIG1 and modulate an immune response. In some instances, these anti-LRIG1 antibodies are used to treat diseases that benefit from the modulation (i.e. either activation or inhibition) of an immune response in a subject (e.g. cancers or immune-related diseases such as autoimmune diseases, inflammatory diseases, or wound healing). In some embodiments, other anti-LRIG1 antibodies and methods of using are described in PCT Publication WO 2019/165233, which is hereby incorporated by reference in its entirety.

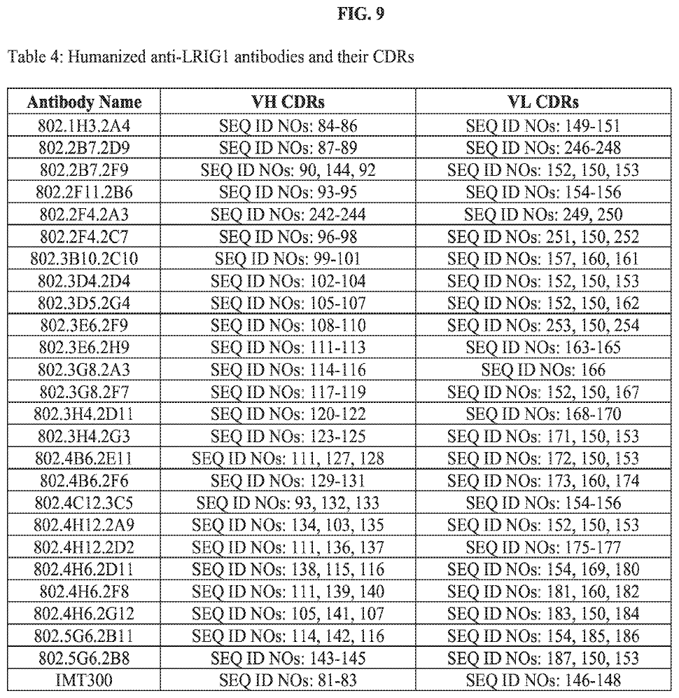

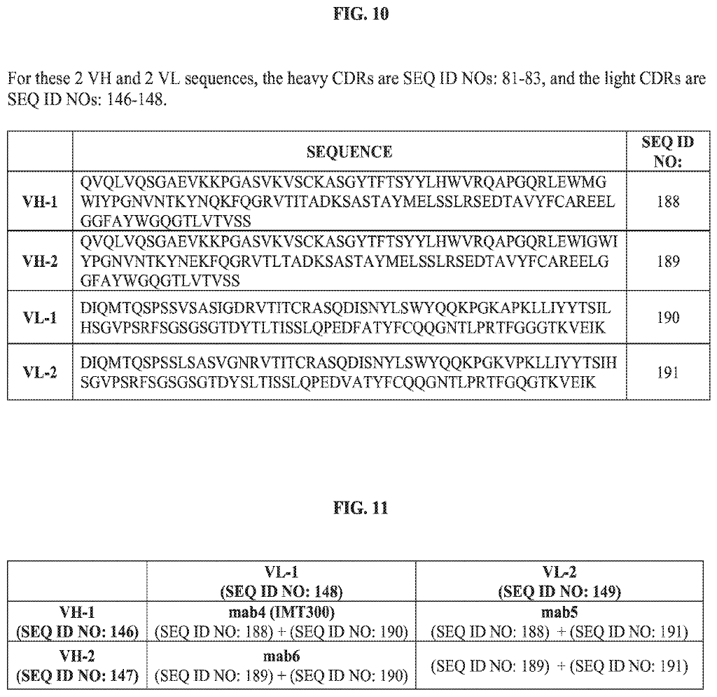

Disclosed herein, in some embodiments, is an antigen binding polypeptide. In some embodiments, the antigen binding polypeptide is an antibody. In some embodiments, the polypeptide or antibody exhibits specific binding to LRIG1 protein (SEQ ID NO: 2). In some embodiments, the antibody or polypeptide binds to an epitope present on one or more regions of LRIG1 selected from a group consisting of any amino acid sequence from amino acid residues from position 1 to 564 or position 655 to 1093 from N terminus to C terminus of LRIG1 protein. In some embodiments, the antibody or polypeptide binds to an epitope present within an amino acid sequence from amino acid residues from position 674 to 714 from N terminus to C terminus of LRIG1 protein. In some embodiments, the antibody or polypeptide binds to an epitope present within an amino acid sequence from amino acid residues from position 704 to 744 from N terminus to C terminus of LRIG1 protein. In some embodiments, the antibody or polypeptide binds to an undetermined epitope of LRIG1 between position 1 to 564 or position 655 to 1093 from N terminus to C terminus. In some embodiments, the antibody or polypeptide comprises a full-length antibody or a fragment thereof. In some embodiments, the antibody or polypeptide comprises a bispecific antibody or a binding fragment thereof. In some embodiments, the antibody or polypeptide comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody or polypeptide comprises at least one complementarity-defining region selected from SEQ ID NOs: 84-145, 149-187, or 242-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or antigen binding polypeptide comprises 3 heavy chain CDRs and 3 light chain CDRs according to FIG. 9. In some embodiments, the antibody or polypeptide comprises an IgA, IgD, IgE, IgG, or IgM framework. In some embodiments, the antibody or polypeptide comprises an IgG framework. In some embodiments, the antibody or polypeptide comprises an IgG1, IgG2, or IgG4 framework. In some embodiments, the antibody or polypeptide comprises a k_{D} of less than 1 nM, 1.2 nM, 2 nM, 5 nM, 10 nM, 13.5 nM, 15 nM, 20 nM, 25 nM, or 30 nM, or any k_{D} to LRIG1 within a range defined by any two of the aforementioned k_{D}. In some embodiments, the antibody or polypeptide comprises a humanized antibody. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antigen binding polypeptide comprises a VH and VL according to Table 5. In some embodiments, the antibody or polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8, or any combination thereof. In some embodiments, the antibody or polypeptide is not IMT300, mab4, mab5, or mab6. In some embodiments, the antibody or polypeptide does not comprise a VH having the sequence of SEQ ID NO: 188 or SEQ ID NO: 189. In some embodiments, the antibody or polypeptide does not comprise a VL having the sequence of SEQ ID NO: 190 or SEQ ID NO: 191.

Disclosed herein, in some embodiments, is an antigen binding polypeptide, wherein the polypeptide disrupts LRIG1-VISTA interaction. In some embodiments, the antigen binding polypeptide is an antibody. In some embodiments, the antibody or polypeptide is any one of the antibodies or polypeptides disclosed herein. In some embodiments, the antibody or polypeptide binds to an epitope of LRIG1 in a region from SEQ ID NO: 69 to 75. In some embodiments, the antibody or polypeptide binds to an epitope present within the region of LRIG1 defined by Peptide 65 (SEQ. ID No. 69). In some embodiments, the antibody or polypeptide binds to an epitope present within the region of LRIG1 defined by Peptide 66 (SEQ. ID No. 70). In some embodiments, the antibody or polypeptide binds to an epitope present within the region of LRIG1 defined by Peptide 67 (SEQ. ID No. 71). In some embodiments, the antibody or polypeptide binds to an epitope present within the region of LRIG1 defined by Peptide 68 (SEQ. ID No. 72). In some embodiments, the antibody or polypeptide binds to an epitope present within the region of LRIG1 defined by Peptide 69 (SEQ. ID No. 73). In some embodiments, the antibody or polypeptide binds to an epitope present within the region of LRIG1 defined by Peptide 70 (SEQ. ID No. 74). In some embodiments, the antibody or polypeptide binds to an epitope present within the region of LRIG1 defined by Peptide 71 (SEQ. ID No. 75). In some embodiments, the antibody or polypeptide comprises a full-length antibody or a fragment thereof. In some embodiments, the antibody or polypeptide comprises a bispecific antibody or a binding fragment thereof. In some embodiments, the antibody or polypeptide comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody or polypeptide comprises at least one complementarity-defining region selected from SEQ ID NOs: 84-145, 149-187, or 242-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or antigen binding polypeptide comprises 3 heavy chain CDRs and 3 light chain CDRs according to FIG. 9. In some embodiments, the antibody or polypeptide comprises an IgA, IgD, IgE, IgG, or IgM framework. In some embodiments, the antibody or polypeptide comprises an IgG framework. In some embodiments, the antibody or polypeptide comprises an IgG1, IgG2, or IgG4 framework. In some embodiments, the antibody or polypeptide comprises a k_{D} of less than 1 nM, 1.2 nM, 2 nM, 5 nM, 10 nM, 13.5 nM, 15 nM, 20 nM, 25 nM, or 30 nM, or any k_{D} to LRIG1 within a range defined by any two of the aforementioned k_{D}. In some embodiments, the antibody or polypeptide comprises a humanized antibody. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antibody or antigen binding polypeptide comprises a VH and VL according to Table 5. In some embodiments, the antibody or polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8, or any combination thereof. In some embodiments, the antibody or polypeptide is not IMT300, mab4, mab5, or mab6. In some embodiments, the antibody or polypeptide does not comprise a VH having the sequence of SEQ ID NO: 188 or SEQ ID NO: 189. In some embodiments, the antibody or polypeptide does not comprise a VL having the sequence of SEQ ID NO: 190 or SEQ ID NO: 191.

Disclosed herein, in some embodiments, is an antigen binding polypeptide, wherein the polypeptide exhibits specific binding to LRIG1 protein. In some embodiments, the antigen binding polypeptide is an antibody. In some embodiments, the antibody or polypeptide is any one of the antibodies or polypeptides disclosed herein. In some embodiments, the binding of the antibody or polypeptide to LRIG1 reduces an interaction between LRIG1-VISTA to less than 21% of interaction between LRIG1-VISTA without the antibody or antigen binding polypeptide. In some embodiments, the binding of the antibody or polypeptide to LRIG1 reduces an interaction between LRIG1-VISTA to less than 20%, less than 15%, less than 10%, less than 5%, or less than 1%, or any reduction within a range defined by any two of the aforementioned percentages, of the interaction between LRIG1-VISTA without the antibody or antigen binding polypeptide. In some embodiments, the antibody or polypeptide binds to an epitope of LRIG1 in a region from SEQ ID NO: 69 to 75. In some embodiments, the antibody or polypeptide comprises a full-length antibody or a fragment thereof. In some embodiments, the antibody or polypeptide comprises a bispecific antibody or a binding fragment thereof. In some embodiments, the antibody or polypeptide comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody or polypeptide comprises at least one complementarity-defining region selected from SEQ ID NOs: 84-145, 149-187, or 242-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or antigen binding polypeptide comprises 3 heavy chain CDRs and 3 light chain CDRs according to FIG. 9. In some embodiments, the antibody or polypeptide comprises an IgA, IgD, IgE, IgG, or IgM framework. In some embodiments, the antibody or polypeptide comprises an IgG framework. In some embodiments, the antibody or polypeptide comprises an IgG1, IgG2, or IgG4 framework. In some embodiments, the antibody or polypeptide comprises a k_{D} of less than 1 nM, 1.2 nM, 2 nM, 5 nM, 10 nM, 13.5 nM, 15 nM, 20 nM, 25 nM, or 30 nM, or any k_{D} to LRIG1 within a range defined by any two of the aforementioned k_{D}. In some embodiments, the antibody or polypeptide comprises a humanized antibody. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antibody or antigen binding polypeptide comprises a VH and VL according to Table 5. In some embodiments, the antibody or polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8, or any combination thereof. In some embodiments, the antibody or polypeptide is not IMT300, mab4, mab5, or mab6. In some embodiments, the antibody or polypeptide does not comprise a VH having the sequence of SEQ ID NO: 188 or SEQ ID NO: 189. In some embodiments, the antibody or polypeptide does not comprise a VL having the sequence of SEQ ID NO: 190 or SEQ ID NO: 191.

Disclosed herein, in some embodiments, is an antigen binding polypeptide, wherein the polypeptide comprises at least one complementarity-defining region selected from SEQ ID NOs: 84-145, 149-187, or 242-254, and wherein the polypeptide is capable of binding an epitope present in one or more regions of LRIG1 protein. In some embodiments, the antigen binding polypeptide is an antibody. In some embodiments, the antibody or polypeptide is any one of the antibodies or polypeptides disclosed herein. In some embodiments, the antibody or polypeptide comprises a full-length antibody or a fragment thereof. In some embodiments, the antibody or polypeptide comprises a bispecific antibody or a binding fragment thereof. In some embodiments, the antibody or polypeptide comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody or polypeptide comprises more than one complementarity-defining region selected from SEQ ID NOs: 84-145, 149-187, or 242-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or antigen binding polypeptide comprises 3 heavy chain CDRs and 3 light chain CDRs according to FIG. 9. In some embodiments, the antibody or polypeptide comprises an IgA, IgD, IgE, IgG, or IgM framework. In some embodiments, the antibody or polypeptide comprises an IgG framework. In some embodiments, the antibody or polypeptide comprises an IgG1, IgG2, or IgG4 framework. In some embodiments, the antibody or polypeptide comprises a k_{D} of less than 1 nM, 1.2 nM, 2 nM, 5 nM, 10 nM, 13.5 nM, 15 nM, 20 nM, 25 nM, or 30 nM, or any k_{D} to LRIG1 within a range defined by any two of the aforementioned k_{D}. In some embodiments, the antibody or polypeptide comprises a humanized antibody. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antibody or antigen binding polypeptide comprises a VH and VL according to Table 5. In some embodiments, the antibody or polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8, or any combination thereof. In some embodiments, the antibody or polypeptide is not IMT300, mab4, mab5, or mab6. In some embodiments, the antibody or polypeptide does not comprise a VH having the sequence of SEQ ID NO: 188 or SEQ ID NO: 189. In some embodiments, the antibody or polypeptide does not comprise a VL having the sequence of SEQ ID NO: 190 or SEQ ID NO: 191.

Disclosed herein, in some embodiments, is an antibody or antigen binding polypeptide, wherein the antibody or polypeptide exhibits specific binding to LRIG1 protein such that upon binding said antibody or polypeptide reduces an interaction between LRIG1-VISTA by (i) at least 60%, 65%, 70%, 75%, 79%, 80%, 85%, 90%, 95%, or 99%, or any reduction within a range defined by any two of the aforementioned percentages, wherein said antibody or polypeptide is not IMT300, mab4, mab5, or mab5, and/or does not comprise a VH having the sequence of SEQ ID NO: 188 or SEQ ID NO: 189. In some embodiments, the antibody or polypeptide does not comprise a VL having the sequence of SEQ ID NO: 190 or SEQ ID NO: 191 or (ii) a greater degree than IMT300, mab4, mab5, or mab5, or an antibody or polypeptide having a VH having the sequence of SEQ ID NO: 188 or SEQ ID NO: 189 and/or a VL having the sequence of SEQ ID NO: 190 or SEQ ID NO: 191. In other embodiments disclosed herein is an antibody or antigen binding polypeptide, wherein the antibody or polypeptide exhibits specific binding to LRIG1 protein such that upon binding said antibody or polypeptide reduces an interaction between LRIG1-VISTA by (i) at least 60%, 65%, 70%, 75%, 79%, 80%, 85%, 90%, 95%, or 99%, or any reduction within a range defined by any two of the aforementioned percentages, wherein said antibody or polypeptide is not IMT300 or (ii) a greater degree than IMT300.

Disclosed herein, in some embodiments, is a complex comprising any of the above-described antibodies or polypeptides or any antibody or polypeptide disclosed herein, wherein the complex comprises the antibody or polypeptide bound to LRIG1 protein.

Disclosed herein, in some embodiments, is a method of disrupting an interaction between LRIG1 and an interacting protein. In some embodiments, the methods comprise contacting a plurality of cells comprising a cell expressing LRIG1, a cell expressing the interacting protein, or a combination thereof with any of the above-described antibodies or antigen binding polypeptides, or any one of the antibodies or antigen binding polypeptides disclosed herein. In some embodiments, the cell expressing LRIG1 and/or the cell expressing the interacting protein is a non-immune cell. In some embodiments, the cell expressing LRIG1 and/or the cell expressing the interacting protein is an immune cell. In some embodiments, disrupting the interaction between LRIG1 and the interacting protein modulates immune function of immune cells, for example, the cell expressing the interacting protein. In some embodiments, the interaction between LRIG1 and the interacting protein is reduced to less than 30%, less than 21%, less than 20%, less than 19%, less than 17%, less than 10%, less than 5%, or less than 1%, or any reduction within a range defined by any two of the aforementioned percentages. In some embodiments, the interaction occurs at one or more residues of LRIG1 selected from region 245-260, wherein the residue positions correspond to positions 245-260 of SEQ ID NO: 2. In some embodiments, the interacting protein is VISTA. In some embodiments, the interaction occurs at one or more residues of VISTA selected from region 78-90 or 68-92, wherein the residue positions correspond to positions 78-90 or 68-92 of SEQ ID NO: 4. In some embodiments, the antibody or polypeptide binds to at least one amino acid residue within any peptide from SEQ ID NO. 69 to 75. In some embodiments, the antibody or polypeptide comprises a k_{D} of less than 1 nM, 1.2 nM, 2 nM, 5 nM, 10 nM, 13.5 nM, 15 nM, 20 nM, 25 nM, or 30 nM, or any k_{D} to LRIG1 within a range defined by any two of the aforementioned k_{D}. In some embodiments, the antibody or polypeptide comprises a humanized antibody. In some embodiments, the antibody or polypeptide comprises a full-length antibody or a binding fragment thereof. In some embodiments, the antibody or polypeptide comprises a bispecific antibody or a binding fragment thereof. In some embodiments, the antibody or polypeptide comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody or polypeptide is a humanized antibody comprising at least one complementarity-determining regions (CDRs) from SEQ ID NOs: 84-145, 149-187, or 242-254. In some embodiments, the humanized antibody comprises a heavy chain variable region (VH) comprising one, two, or three CDR sequences selected from SEQ ID NOs: 84 to 145 or 242-245. In some embodiments, the humanized antibody comprises a light chain variable region (VL) comprising one, two, or three CDR sequences selected from SEQ ID NOs: 149 to 187 or 246-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or antigen binding polypeptide comprises 3 heavy chain CDRs and 3 light chain CDRs according to FIG. 9. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antibody or antigen binding polypeptide comprises a VH and VL according to Table 5. In some embodiments, the antibody is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8, or any combination thereof. In some embodiments, the antibody or polypeptide is not IMT300, mab4, mab5, or mab6. In some embodiments, the antibody or polypeptide does not comprise a VH having the sequence of SEQ ID NO: 188 or SEQ ID NO: 189. In some embodiments, the antibody or polypeptide does not comprise a VL having the sequence of SEQ ID NO: 190 or SEQ ID NO: 191. In some embodiments, the antibody or polypeptide comprises an IgA, IgD, IgE, IgG, or IgM framework. In some embodiments, the antibody or polypeptide comprises an IgG framework. In some embodiments, the antibody or polypeptide comprises an IgG1, IgG2, or IgG4 framework.

Disclosed herein, in some embodiments, is a method of disrupting an interaction between VISTA and LRIG1. In some embodiments, the methods comprise contacting a plurality of cells comprising a LRIG1-expressing cell, a VISTA-expressing cell, or a combination thereof with any of the above-described antibodies or antigen binding polypeptides, or any one of the antibodies or antigen binding polypeptides disclosed herein. In some embodiments, the LRIG1-expressing cell and/or the VISTA-expressing cell is a non-immune cell. In some embodiments, the LRIG1-expressing cell and/or the VISTA-expressing cell is an immune cell. In some embodiments, disrupting the interaction between VISTA and LRIG1 modulates immune function of immune cells, for example, the VISTA-expressing cell. In some embodiments, the LRIG1-VISTA interaction is reduced to less than 30%, less than 21%, less than 20%, less than 19%, less than 17%, less than 10%, less than 5%, or less than 1%, or any reduction within a range defined by any two of the aforementioned percentages. In some embodiments, the interaction occurs at one or more residues of LRIG1 selected from region 245-260, wherein the residue positions correspond to positions 245-260 of SEQ ID NO: 2. In some embodiments, the interaction occurs at one or more residues of VISTA selected from region 78-90 or 68-92, wherein the residue positions correspond to positions 78-90 or 68-92 of SEQ ID NO: 4. In some embodiments, the antibody or polypeptide binds to at least one amino acid residue within any peptide from SEQ ID NO. 69 to 75. In some embodiments, the antibody or polypeptide comprises a k_{D} of less than 1 nM, 1.2 nM, 2 nM, 5 nM, 10 nM, 13.5 nM, 15 nM, 20 nM, 25 nM, or 30 nM, or any k_{D} to LRIG1 within a range defined by any two of the aforementioned k_{D}. In some embodiments, the antibody or polypeptide comprises a humanized antibody. In some embodiments, the antibody or polypeptide comprises a full-length antibody or a binding fragment thereof. In some embodiments, the antibody or polypeptide comprises a bispecific antibody or a binding fragment thereof. In some embodiments, the antibody or polypeptide comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody or polypeptide is a humanized antibody comprising at least one complementarity-determining regions (CDRs) from SEQ ID NOs: 84-145, 149-187, or 242-254. In some embodiments, the humanized antibody comprises a heavy chain variable region (VH) comprising one, two, or three CDR sequences selected from SEQ ID NOs: 84 to 145 or 242-245. In some embodiments, the humanized antibody comprises a light chain variable region (VL) comprising one, two, or three CDR sequences selected from SEQ ID NOs: 149 to 187 or 246-254. In some embodiments, the antibody or polypeptide comprises at least one heavy chain CDR selected from SEQ ID NOs: 84-145 or 242-245. In some embodiments, the antibody or polypeptide comprises at least one light chain CDR selected from SEQ ID NOs: 147-187 or 246-254. In some embodiments, the antibody or antigen binding polypeptide comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody or antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody or antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody or antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody or antigen binding polypeptide comprises at least one light chain CDR2 selected from the group consisting of SEQ ID NOs: 150, 155, 158, 160, 164, 169, 176, 179, 185, 247, and 249. In some embodiments, the antibody or antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody or antigen binding polypeptide comprises 3 heavy chain CDRs and 3 light chain CDRs according to FIG. 9. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the antibody or antigen binding polypeptide comprises a VH and VL according to Table 5. In some embodiments, the antibody or polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8, or any combination thereof. In some embodiments, the antibody or polypeptide is not IMT300, mab4, mab5, or mab6. In some embodiments, the antibody or polypeptide does not comprise a VH having the sequence of SEQ ID NO: 188 or SEQ ID NO: 189. In some embodiments, the antibody or polypeptide does not comprise a VL having the sequence of SEQ ID NO: 190 or SEQ ID NO: 191. In some embodiments, the antibody or polypeptide comprises an IgA, IgD, IgE, IgG, or IgM framework. In some embodiments, the antibody or polypeptide comprises an IgG framework. In some embodiments, the antibody or polypeptide comprises an IgG1, IgG2, or IgG4 framework.

Also disclosed herein in some embodiments are methods of modulating an immune response. In some embodiments, the modulating is performed in a subject. In some embodiments, the subject is a mammal. In some embodiments, the subject is a human. The methods comprise administering to the subject an antibody or antigen binding polypeptide that specifically binds to an LRIG1 protein (having the sequence of SEQ ID NO: 2). In some embodiments, the antibody or antigen binding polypeptide is any one of the antibodies or antigen binding polypeptides disclosed herein. In some embodiments, the antibody or antigen binding polypeptide disrupts an interaction between LRIG1 and an interacting protein. In some embodiments, disrupting the interaction between LRIG1 and the interacting protein occurs according to any one of the disruption methods disclosed herein. In some embodiments, the interacting protein is VISTA. In some embodiments, modulating the immune response comprises enhancing the level of T-cell-mediated and/or B-cell-mediated immune response in the subject. In some embodiments, modulating the immune response comprises reducing the level of T-cell-mediated and/or B-cell-mediated immune response, or increasing the immunosuppressive function of regulatory T cells, or both, in the subject. In some embodiments, the subject comprises a cancer. In some embodiments, modulating the immune response ameliorates, treats, or reduces symptoms of the cancer. In some embodiments, the cancer is any one of the cancers disclosed herein. In some embodiments, the cancer is breast cancer, colorectal cancer, kidney cancer, liver cancer, lung cancer, brain cancer, pancreatic cancer, bladder cancer, or stomach cancer, or a hematological malignancy, or any combination thereof. In some embodiments, the subject comprises an immune-related disorder. In some embodiments, modulating the immune response ameliorates, treats, or reduces symptoms of the immune-related disorder or condition. In some embodiments, the immune-related disorder or condition comprises an autoimmune disease, an inflammatory disease, or wound healing. In some embodiments, the immune-related disorder is fibrosis, liver fibrosis, kidney fibrosis, cardiac fibrosis, pulmonary fibrosis, non-alcoholic fatty liver disease, non-alcoholic steatohepatitis, sepsis, atopic dermatitis, psoriasis, or any combination thereof. In some embodiments, the methods further comprise administering an additional therapeutic agent to the subject. In some embodiments, the additional therapeutic agent comprises an immunotherapeutic agent, an immune checkpoint modulator, a chemotherapeutic agent, targeted therapeutic agent, hormonal therapeutic agent, or a stem cell-based therapeutic agent. In some embodiments, the additional therapeutic agent is any one of the therapeutic agents disclosed herein or known in the art. In some embodiments, the antibody or antigen binding polypeptide is administered parenterally. In some embodiments, the antibody or antigen binding polypeptide is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8, or any combination thereof. In some embodiments, the antibody or polypeptide is not IMT300, mab4, mab5, or mab6. In some embodiments, the antibody or polypeptide does not comprise a VH having the sequence of SEQ ID NO: 188 or SEQ ID NO: 189. In some embodiments, the antibody or polypeptide does not comprise a VL having the sequence of SEQ ID NO: 190 or SEQ ID NO: 191.

Also disclosed herein in some embodiments are antibodies or antigen binding polypeptides for use in the treatment of a cancer in a subject in need thereof. In some embodiments, the antibodies or antigen binding polypeptides are any of the antibodies or antigen binding polypeptides disclosed herein. In some embodiments, the cancer is breast cancer, colorectal cancer, kidney cancer, liver cancer, lung cancer, brain cancer, pancreatic cancer, bladder cancer, or stomach cancer, or a hematological malignancy, or any combination thereof.

Also disclosed herein in some embodiments are antibodies or antigen binding polypeptides for use in the treatment of an immune-related disorder in a subject in need thereof. In some embodiments, the antibodies or antigen binding polypeptides are any of the antibodies or antigen binding polypeptides disclosed herein. In some embodiments, the immune-related disorder is fibrosis, liver fibrosis, kidney fibrosis, cardiac fibrosis, pulmonary fibrosis, non-alcoholic fatty liver disease, non-alcoholic steatohepatitis, sepsis, atopic dermatitis, psoriasis, or any combination thereof. In some embodiments, the subject is a mammal. In some embodiments, the subject is a human.

Also disclosed herein in some embodiments are pharmaceutical compositions. In some embodiments, the pharmaceutical compositions comprise any one of the antibodies or antigen binding polypeptides disclosed herein and at least one pharmaceutically acceptable carrier, excipient, diluent, or adjuvant. In some embodiments, the at least one pharmaceutically acceptable carrier, excipient, diluent, or adjuvant is any one of the pharmaceutically acceptable carriers, excipients, diluents, or adjuvants disclosed herein or known in the art.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, e.g., within 15%, 10%, or 5%.

The section headings used herein are for organizational purposes only and are not to be construed as limiting the subject matter described.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “individual(s)”, “subject(s)” and “patient(s)” mean any mammal. In some embodiments, the mammal is a human. In some embodiments, the mammal is a non-human None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly and can be modified to reduce their antigenicity.

In addition to entire immunoglobulins (or their recombinant counterparts), immunoglobulin fragments or “binding fragments” comprising the epitope binding site (e.g., Fab′, F(ab′)₂, single-chain variable fragment (scFv), diabody, minibody, nanobody, single-domain antibody (sdAb), or other fragments) are useful as antibody moieties in the present invention. Such antibody fragments may be generated from whole immunoglobulins by ricin, pepsin, papain, or other protease cleavage. Minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g. pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (e.g. U.S. Pat. No. 5,985,660, hereby expressly incorporated by reference in its entirety).

The term “humanized” as applies to a non-human (e.g. rodent or primate) antibodies are hybrid immunoglobulins, immunoglobulin chains or fragments thereof which contain minimal sequence derived from non-human immunoglobulin.

As used herein, the terms “treating” or “treatment” (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the patient. The length of the treatment period depends on a variety of factors, such as the severity of the condition, the age and genetic profile of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The terms “effective amount” or “effective dose” as used herein have their plain and ordinary meaning as understood in light of the specification, and refer to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

The term “immune cells” refers to cells of hematopoietic origin that are involved in the specific recognition of antigens Immune cells include antigen presenting cells (APCs), such as dendritic cells or macrophages, B cells, T cells, helper T cells, CD4+ T cells, cytotoxic T cells, CD8+ T cells, regulatory T Cells (Treg), natural killer cells, immature myeloid cells, and myeloid cells, such as monocytes, macrophages, eosinophils, mast cells, basophils, and granulocytes.

The term “immune response” refers to, for example, T cell-mediated and/or B cell-mediated immune responses. Exemplary immune responses include B cell responses (e.g., antibody production) T cell responses (e.g., cytokine production, and cellular cytotoxicity) and activation of cytokine responsive cells, e.g., macrophages. Modulation of an immune response includes either activating the immune response, or inhibiting the immune response. Activating the immune response refers to enhancing the level of T-cell-mediated and/or B cell-mediated immune response, using methods disclosed herein or known to one of skilled in the art. In one embodiment, the level of enhancement is at least 20-50%, alternatively at least 60%, at least 70%, at least 80%, at least 90%, at least 100%, at least 120%, at least 150%, or at least 200%. Inhibiting the immune response refers to reducing the level of T-cell-mediated and/or B cell-mediated immune response, or increasing the immunosuppressive function of regulatory T cells, using methods disclosed herein or known to one of skilled in the art. In one embodiment, the level of immune response reduction or increase of regulatory T cell function is at least 20-50%, alternatively at least 60%, at least 70%, at least 80%, at least 90%, at least 100%, at least 120%, at least 150%, or at least 200%.

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy.

The term “% w/w” or “% wt/wt” means a percentage expressed in terms of the weight of the ingredient or agent over the total weight of the composition multiplied by 100.

### Methods of Use

In some embodiments disclosed herein are methods of modulating an immune response. In some embodiments, the methods comprise contacting an anti-LRIG1 antibody or antigen binding polypeptide to a plurality of cells comprising a cell expressing LRIG1, a cell expressing an interacting protein, or a combination thereof. In some embodiments, the interacting protein is VISTA. In some embodiments, the cell expressing LRIG1 is an LRIG-expressing cell. In some embodiments, the cell expressing an interacting protein is a VISTA-expressing cell. In some embodiments, the anti-LRIG1 antibody or antigen binding polypeptide is any one of the antibodies or polypeptides disclosed herein. In some embodiments, contacting the anti-LRIG1 antibody or antigen binding polypeptide induces immune activation of the cell expressing LRIG1, the cell expressing an interacting protein, or both. In some embodiments, contacting the anti-LRIG1 antibody or antigen binding polypeptide inhibits immune activation of the cell expressing LRIG1, the cell expressing an interacting protein, or both. In some embodiments, the methods are performed on a subject. In some embodiments, the methods comprise administering the anti-LRIG1 antibody or antigen binding polypeptide to the subject. In some embodiments, the subject is a mammal. In some embodiments, the subject is a human. In some embodiments, administering the anti-LRIG1 antibody or antigen binding polypeptide to the subject induces immune activation in the subject. In some embodiments, immune activation in the subject comprises upregulation of activity of immune cells (e.g. B cells and/or cytotoxic T cells). In some embodiments, administering the anti-LRIG1 antibody or antigen binding polypeptide to the subject inhibits immune activation in the subject. In some embodiments, inhibition of immune activation in the subject comprises downregulation of activity of immune cells, or upregulation of the proliferation and/or function of immunosuppressive cells (e.g. regulatory T cells) in the subject.

In some cases, the LRIG1-expressing cell upon binding to the anti-LRIG1 antibody or polypeptide expresses a cytokine which induces immune activation and/or modulates immune function. In some embodiments, modulation of immune function comprises any one of the embodiments of modulation of immune function disclosed herein (e g immune activation, or inhibition of immune function). In some embodiments, the cytokine is a chemokine, an interferon, an interleukin, a lymphokine, a monokine, a tumor necrosis factor, CCL1, CCl2, CCL3, CCL4, CCL5, CCL6, CCL7, CCL8, CCL9, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CX3CL1, XCL1, XCL2, INFα, INFβ, INFγ, IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-17A-F, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, adesleukin, GM-CSF, TNFα, TNFβ, TNFγ, TGF-I-3 TNFSF4, TNFSF5, TNFSF6, TNFSF7, TNFSF8, TNFSF9, TNFSF10, TNFSF11, TNFSF12, TNFSF13, TNFSF13B, TNFSF14, TNFSF15, TNFSF18, or TNFSF19, leukemia inhibitor factor (LIF), ciliary neurotrophic factor (CNTF), CNTF-like cytokine (CLC), cardiotrophin (CT), Kit ligand (KL), or any combination thereof. In some cases, the cytokine is an interferon. In some cases, the interferon is IFNγ. In some cases, the cytokine is an interleukin. In some cases, the interleukin is IL-2. In some cases, the cytokine production is 110%, 120%, 130%, 140%, 150%, 160%, 170%, 180%, 190%, 200%, 300%, 400%, 500%, 600%, or more of cytokine production by an isotype antibody, or any increase within a range defined by any two of the aforementioned percentages. In some cases, the cytokine production is 150% of cytokine production by an isotype antibody. In some cases, the cytokine production is 160% of cytokine production by an isotype antibody. In some cases, the cytokine production is 170% of cytokine production by an isotype antibody. In some cases, the cytokine production is 180% of cytokine production by an isotype antibody. In some cases, the cytokine production is 190% of cytokine production by an isotype antibody. In some cases, the cytokine production is 200% of cytokine production by an isotype antibody. In some cases, the cytokine production is more than 200% of cytokine production by an isotype antibody. In some cases, the cytokine production is more than 300% of cytokine production by an isotype antibody. In some cases, the cytokine production is more than 400% of cytokine production by an isotype antibody. In some cases, the cytokine production is more than 500% of cytokine production by an isotype antibody.

In some cases, the modulation of immune function comprises a proliferation of CD3+ T lymphocytes, CD4+ T helper cells, CD8+ cytotoxic T cells, B cells, Natural Killer (NK) cells, Tregs, or any combination thereof. In some cases, the modulation of immune function comprises a proliferation of CD3+ T lymphocytes. In some cases, the modulation of immune function comprises a proliferation of CD4+ T helper cells. In some cases, the modulation of immune function comprises a proliferation of CD8+ cytotoxic T cells. In some cases, the modulation of immune function, including, in some situations, immune activation comprises a proliferation of B cells. In some cases, the modulation of immune function (which can include, in some situations, immune activation and other modulations of immune activity) comprises a proliferation of NK cells. In some cases, the modulation of immune function (such as, in some situations, immune activation) comprises a proliferation of B cells and NK cells and/or Tregs. In some embodiments, the proliferation of cells is within any one of the plurality of cells disclosed herein, or in any one of the subjects disclosed herein.

In some embodiments, the modulation of immune function comprises a decrease in CD3+ T lymphocytes, CD4+ T helper cells, CD8+ cytotoxic T cells, B cells, NK cells, or any combination thereof. In some embodiments, the modulation of immune function comprises a decrease in CD3+ T lymphocytes. In some embodiments, the modulation of immune functions comprises a decrease in CD4+ T helper cells. In some embodiments, the modulation of immune function comprises a decrease in CD8+ cytotoxic T cells. In some embodiments, the modulation of immune function comprises a decrease in B cells. In some embodiments, the modulation of immune function comprises a decrease in NK cells. In some embodiments, the modulation of immune function comprises a decrease in B cells and NK cells. In some embodiments, the decrease in cells is within any one of the plurality of cells disclosed herein, or in any one of the subjects disclosed herein.

In some cases, the modulation of immune function comprises an increase in M1 macrophage population within any one of the plurality of cells or subjects disclosed herein. In some embodiments, the modulation of immune function comprises a decrease in M1 macrophage population within any one of the plurality of cells or subjects disclosed herein. In some cases, the modulation of immune function comprises a decrease in M2 macrophage population within any one of the plurality of cells or subjects disclosed herein. In some embodiments, the modulation of immune function comprises an increase in M2 macrophage population within any one of the plurality of cells or subjects disclosed herein. In some cases, the modulation of immune function comprises an increase in M1 macrophage population and a decrease in M2 macrophage population within any one of the plurality of cells or subjects disclosed herein. In some embodiments, the modulation of immune function comprises a decrease in M1 macrophage population and an increase in M2 macrophage population within any one of the plurality of cells or subjects disclosed herein.

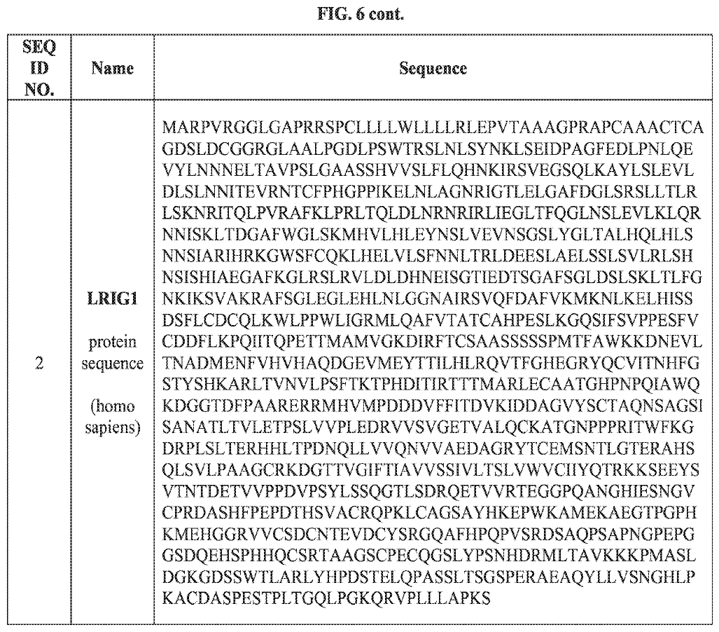

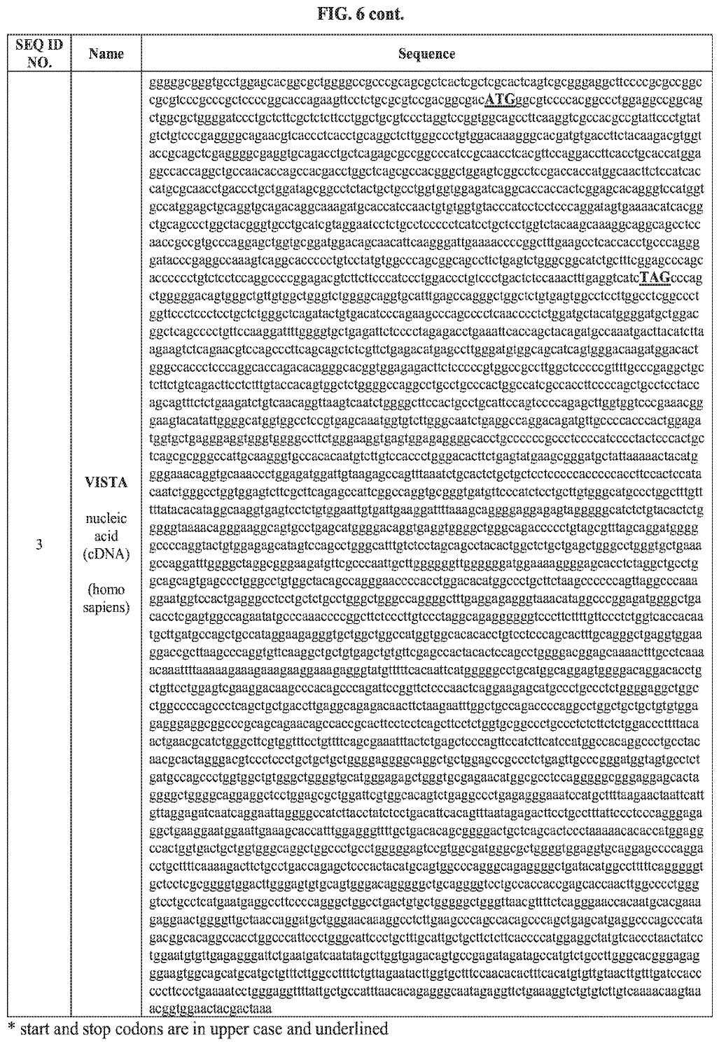

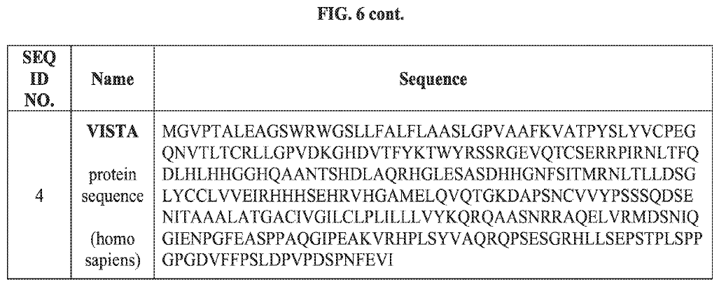

FIG. 6 illustrates sequences LRIG1 and VISTA.

In some cases, an anti-LRIG1 antibody or polypeptide binds to LRIG1 and disrupts an interaction between LRIG1 and an interacting protein. In some embodiments, the anti-LRIG1 antibody or polypeptide is any one of the antibodies or polypeptides disclosed herein. In some cases, disruption of an interaction between LRIG1 and the interacting protein includes partial inhibition of interaction between LRIG1 and the interacting protein. In some cases, disruption of an interaction between LRIG1 and the interacting protein includes complete inhibition of interaction between LRIG1 and the interacting protein. In some cases, the anti-LRIG1 antibody or polypeptide binds to LRIG1 and reduces an interaction between LRIG1 and the interacting protein. In some cases, the interaction between LRIG1 and the interacting protein is reduced to less than 80%, less than 78%, less than 70%, less than 72%, less than 66%, less than 60%, less than 56%, less than 54%, less than 52%, less than 50%, less than 44%, less than 43%, less than 40%, less than 30%, less than 29%, less than 27%, less than 21%, less than 20%, less than 19%, less than 17%, less than 10%, less than 5%, or less than 1%, or any reduction within a range defined by any two of the aforementioned percentages. In some cases, the interaction between LRIG1 and the interacting protein is reduced to less than 70%. In some cases, the interaction between LRIG1 and the interacting protein is reduced to less than 60%. In some cases, the interaction between LRIG1 and the interacting protein is reduced to less than 59%. In some cases, the interaction between LRIG1 and the interacting protein is reduced to less than 50%. In some cases, the interaction between LRIG1 and the interacting protein is reduced to less than 44%. In some cases, the interaction between LRIG1 and the interacting protein is reduced to less than 43%. In some cases, the interaction between LRIG1 and the interacting protein is reduced to less than 40%. In some cases, the interaction between LRIG1 and the interacting protein is reduced to less than 34%. In some cases, the interaction between LRIG1 and the interacting protein is reduced to less than 30%. In some cases, the interaction between LRIG1 and the interacting protein is reduced to less than 21%. In some cases, the interaction between LRIG1 and the interacting protein is reduced to less than 20%. In some cases, the interaction between LRIG1 and the interacting protein is reduced to less than 14%. In some cases, the interaction between LRIG1 and the interacting protein is reduced to less than 10%. In some cases, the interaction between LRIG1 and the interacting protein is reduced to less than 7%. In some cases, the interaction between LRIG1 and the interacting protein is reduced to less than 5%. In some cases, the interaction between LRIG1 and the interacting protein is reduced to less than 4%. In some cases, the interaction between LRIG1 and the interacting protein is reduced to less than 1%.

In some cases, the anti-LRIG1 antibody or polypeptide binds to LRIG1 and enhances an interaction between LRIG1 and an interacting protein. In some cases, the interaction between LRIG1 and the interacting protein is enhanced by at least 80%, at least 78%, at least 72%, at least 70%, at least 66%, at least 60%, at least 56%, at least 54%, at least 52%, at least 50%, at least 44%, at least 43%, at least 40%, at least 30%, at least 29%, at least 27%, at least 21%, at least 20%, at least 19%, at least 17%, at least 10%, at least 5%, or at least 1%, or any enhancement within a range defined by any two of the aforementioned percentages. In some embodiments, the interaction between LRIG1 and the interacting protein is enhanced by at least 70%. In some embodiments, the interaction between LRIG1 and the interacting protein is enhanced by at least 60%. In some embodiments, the interaction between LRIG1 and the interacting protein is enhanced by at least 59%. In some embodiments, the interaction between LRIG1 and the interacting protein is enhanced by at least 50%. In some embodiments, the interaction between LRIG1 and the interacting protein is enhanced by at least 44%. In some embodiments, the interaction between LRIG1 and the interacting protein is enhanced by at least 43%. In some embodiments, the interaction between LRIG1 and the interacting protein is enhanced by at least 40%. In some embodiments, the interaction between LRIG1 and the interacting protein is enhanced by at least 34%. In some embodiments, the interaction between LRIG1 and the interacting protein is enhanced by at least 30%. In some embodiments, the interaction between LRIG1 and the interacting protein is enhanced by at least 21%. In some embodiments, the interaction between LRIG1 and the interacting protein is enhanced by at least 20%. In some embodiments, the interaction between LRIG1 and the interacting protein is enhanced by at least 14%. In some embodiments, the interaction between LRIG1 and the interacting protein is enhanced by at least 10%. In some embodiments, the interaction between LRIG1 and the interacting protein is enhanced by at least 7%. In some embodiments, the interaction between LRIG1 and the interacting protein is enhanced by at least 5%. In some embodiments, the interaction between LRIG1 and the interacting protein is enhanced by at least 1%.

In some cases, an anti-LRIG1 antibody or polypeptide binds to LRIG1 and disrupts an interaction between VISTA and LRIG1. In some embodiments, the anti-LRIG1 antibody or polypeptide is any one of the antibodies or polypeptides disclosed herein. In some cases, disruption of an interaction between VISTA and LRIG1 includes partial inhibition of interaction between VISTA and LRIG1. In some cases, disruption of an interaction between VISTA and LRIG1 includes complete inhibition of interaction between VISTA and LRIG1. In some cases, the anti-LRIG1 antibody or polypeptide binds to LRIG1 and reduces an interaction between VISTA and LRIG1. In some cases, the VISTA-LRIG1 interaction is reduced to less than 80%, less than 78%, less than 70%, less than 72%, less than 66%, less than 60%, less than 56%, less than 54%, less than 52%, less than 50%, less than 44%, less than 43%, less than 40%, less than 30%, less than 29%, less than 27%, less than 21%, less than 20%, less than 19%, less than 17%, less than 10%, less than 5%, or less than 1%, or any reduction within a range defined by any two of the aforementioned percentages. In some cases, the LRIG1-VISTA interaction is reduced to less than 70%. In some cases, the VISTA-LRIG1 interaction is reduced to less than 60%. In some cases, the VISTA-LRIG1 interaction is reduced to less than 59%. In some cases, the VISTA-LRIG1 interaction is reduced to less than 50%. In some cases, the VISTA-LRIG1 interaction is reduced to less than 44%. In some cases, the VISTA-LRIG1 interaction is reduced to less than 43%. In some cases, the VISTA-LRIG1 interaction is reduced to less than 40%. In some cases, the VISTA-LRIG1 interaction is reduced to less than 34%. In some cases, the VISTA-LRIG1 interaction is reduced to less than 30%. In some cases, the VISTA-LRIG1 interaction is reduced to less than 21%. In some cases, the VISTA-LRIG1 interaction is reduced to less than 20%. In some cases, the VISTA-LRIG1 interaction is reduced to less than 14%. In some cases, the VISTA-LRIG1 interaction is reduced to less than 10%. In some cases, the VISTA-LRIG1 interaction is reduced to less than 7%. In some cases, the VISTA-LRIG1 interaction is reduced to less than 5%. In some cases, the VISTA-LRIG1 interaction is reduced to less than 4%. In some cases, the VISTA-LRIG1 interaction is reduced to less than 1%.

In some cases, the anti-LRIG1 antibody or polypeptide binds to LRIG1 and enhances an interaction between VISTA and LRIG1. In some cases, the VISTA-LRIG1 interaction is enhanced by at least 80%, at least 78%, at least 72%, at least 70%, at least 66%, at least 60%, at least 56%, at least 54%, at least 52%, at least 50%, at least 44%, at least 43%, at least 40%, at least 30%, at least 29%, at least 27%, at least 21%, at least 20%, at least 19%, at least 17%, at least 10%, at least 5%, or at least 1%, or any enhancement within a range defined by any two of the aforementioned percentages. In some embodiments, the LRIG1-VISTA interaction is enhanced by at least 70%. In some embodiments, the LRIG1-VISTA interaction is enhanced by at least 60%. In some embodiments, the LRIG1-VISTA interaction is enhanced by at least 59%. In some embodiments, the LRIG1-VISTA interaction is enhanced by at least 50%. In some embodiments, the LRIG1-VISTA interaction is enhanced by at least 44%. In some embodiments, the LRIG1-VISTA interaction is enhanced by at least 43%. In some embodiments, the LRIG1-VISTA interaction is enhanced by at least 40%. In some embodiments, the LRIG1-VISTA interaction is enhanced by at least 34%. In some embodiments, the LRIG1-VISTA interaction is enhanced by at least 30%. In some embodiments, the LRIG1-VISTA interaction is enhanced by at least 21%. In some embodiments, the LRIG1-VISTA interaction is enhanced by at least 20%. In some embodiments, the LRIG1-VISTA interaction is enhanced by at least 14%. In some embodiments, the LRIG1-VISTA interaction is enhanced by at least 10%. In some embodiments, the LRIG1-VISTA interaction is enhanced by at least 7%. In some embodiments, the LRIG1-VISTA interaction is enhanced by at least 5%. In some embodiments, the LRIG1-VISTA interaction is enhanced by at least 1%. In some cases, the interaction between VISTA and LRIG1 occurs at one or more residues of LRIG1 selected from positions 245-260, wherein the residue positions correspond to amino acid residues 245-260 of SEQ ID NO: 2 as read N-terminus to C-terminus. In some cases, the interaction between VISTA and LRIG1 occurs at residue 245, wherein the residue position corresponds to position 245 of SEQ ID NO: 2. In some cases, the interaction between VISTA and LRIG1 occurs at residue 246, wherein the residue position corresponds to position 246 of SEQ ID NO: 2. In some cases, the interaction between VISTA and LRIG1 occurs at residue 247, wherein the residue position corresponds to position 247 of SEQ ID NO: 2. In some cases, the interaction between VISTA and LRIG1 occurs at residue 248, wherein the residue position corresponds to position 248 of SEQ ID NO: 2. In some cases, the interaction between VISTA and LRIG1 occurs at residue 249, wherein the residue position corresponds to position 249 of SEQ ID NO: 2. In some cases, the interaction between VISTA and LRIG1 occurs at residue 250, wherein the residue position corresponds to position 250 of SEQ ID NO: 2. In some cases, the interaction between VISTA and LRIG1 occurs at residue 251, wherein the residue position corresponds to position 251 of SEQ ID NO: 2. In some cases, the interaction between VISTA and LRIG1 occurs at residue 252, wherein the residue position corresponds to position 252 of SEQ ID NO: 2. In some cases, the interaction between VISTA and LRIG1 occurs at residue 253, wherein the residue position corresponds to position 253 of SEQ ID NO: 2. In some cases, the interaction between VISTA and LRIG1 occurs at residue 254, wherein the residue position corresponds to position 254 of SEQ ID NO: 2. In some cases, the interaction between VISTA and LRIG1 occurs at residue 255, wherein the residue position corresponds to position 255 of SEQ ID NO: 2. In some cases, the interaction between VISTA and LRIG1 occurs at residue 256, wherein the residue position corresponds to position 256 of SEQ ID NO: 2. In some cases, the interaction between VISTA and LRIG1 occurs at residue 257, wherein the residue position corresponds to position 257 of SEQ ID NO: 2. In some cases, the interaction between VISTA and LRIG1 occurs at residue 258, wherein the residue position corresponds to position 258 of SEQ ID NO: 2. In some cases, the interaction between VISTA and LRIG1 occurs at residue 259, wherein the residue position corresponds to position 259 of SEQ ID NO: 2. In some cases, the interaction between VISTA and LRIG1 occurs at residue 260, wherein the residue position corresponds to position 260 of SEQ ID NO: 2. In some cases, LRIG1 is human LRIG1.

In some cases, the interaction between LRIG1 and VISTA occurs at one or more residues of VISTA selected from region 78-90 or 68-92, wherein the residue positions correspond to positions 78-90 or 68-92 of SEQ ID NO: 4. In some cases, the interaction between LRIG1 and VISTA occurs at one or more residues of VISTA from region 78-90, wherein the residue positions correspond to positions 78-90 of SEQ ID NO: 4. In some cases, the interaction between LRIG1 and VISTA occurs at one or more residues of VISTA from region 68-92, wherein the residue positions correspond to positions 68-92 of SEQ ID NO: 4. In some cases, the interaction between LRIG1 and VISTA occurs at residue 68, wherein the residue position corresponds to positions 68 of SEQ ID NO: 4. In some cases, the interaction between LRIG1 and VISTA occurs at residue 69, wherein the residue position corresponds to positions 69 of SEQ ID NO: 4. In some cases, the interaction between LRIG1 and VISTA occurs at residue 70, wherein the residue position corresponds to positions 70 of SEQ ID NO: 4. In some cases, the interaction between LRIG1 and VISTA occurs at residue 71, wherein the residue position corresponds to positions 71 of SEQ ID NO: 4. In some cases, the interaction between LRIG1 and VISTA occurs at residue 72, wherein the residue position corresponds to positions 72 of SEQ ID NO: 4. In some cases, the interaction between LRIG1 and VISTA occurs at residue 73, wherein the residue position corresponds to positions 73 of SEQ ID NO: 4. In some cases, the interaction between LRIG1 and VISTA occurs at residue 74, wherein the residue position corresponds to positions 74 of SEQ ID NO: 4. In some cases, the interaction between LRIG1 and VISTA occurs at residue 75, wherein the residue position corresponds to positions 75 of SEQ ID NO: 4. In some cases, the interaction between LRIG1 and VISTA occurs at residue 76, wherein the residue position corresponds to positions 76 of SEQ ID NO: 4. In some cases, the interaction between LRIG1 and VISTA occurs at residue 77, wherein the residue position corresponds to positions 77 of SEQ ID NO: 4. In some cases, the interaction between LRIG1 and VISTA occurs at residue 78, wherein the residue position corresponds to positions 78 of SEQ ID NO: 4. In some cases, the interaction between LRIG1 and VISTA occurs at residue 79, wherein the residue position corresponds to positions 79 of SEQ ID NO: 4. In some cases, the interaction between LRIG1 and VISTA occurs at residue 80, wherein the residue position corresponds to positions 80 of SEQ ID NO: 4. In some cases, the interaction between LRIG1 and VISTA occurs at residue 81, wherein the residue position corresponds to positions 81 of SEQ ID NO: 4. In some cases, the interaction between LRIG1 and VISTA occurs at residue 82, wherein the residue position corresponds to positions 82 of SEQ ID NO: 4. In some cases, the interaction between LRIG1 and VISTA occurs at residue 83, wherein the residue position corresponds to positions 83 of SEQ ID NO: 4. In some cases, the interaction between LRIG1 and VISTA occurs at residue 84, wherein the residue position corresponds to positions 84 of SEQ ID NO: 4. In some cases, the interaction between LRIG1 and VISTA occurs at residue 85, wherein the residue position corresponds to positions 85 of SEQ ID NO: 4. In some cases, the interaction between LRIG1 and VISTA occurs at residue 86, wherein the residue position corresponds to positions 86 of SEQ ID NO: 4. In some cases, the interaction between LRIG1 and VISTA occurs at residue 87, wherein the residue position corresponds to positions 87 of SEQ ID NO: 4. In some cases, the interaction between LRIG1 and VISTA occurs at residue 88, wherein the residue position corresponds to positions 88 of SEQ ID NO: 4. In some cases, the interaction between LRIG1 and VISTA occurs at residue 89, wherein the residue position corresponds to positions 89 of SEQ ID NO: 4. In some cases, the interaction between LRIG1 and VISTA occurs at residue 90, wherein the residue position corresponds to positions 90 of SEQ ID NO: 4. In some cases, VISTA is human VISTA.

In further embodiments, disclosed herein, are methods of promoting or inhibiting B cell or Natural Killer (NK) cell proliferation, comprising contacting a plurality of cells comprising B cells, NK cells, VISTA-expressing cells, and LRIG1-expressing cells with an anti-LRIG1 antibody or polypeptide for a time sufficient to promote proliferation or inhibition of B cells or NK cells in the plurality of cells. In some embodiments, disclosed herein, are methods of promoting or inhibiting B cell and Natural Killer (NK) cell proliferation, comprising contacting a plurality of cells comprising B cells, NK cells, LRIG1-expressing cells, and VISTA-expressing cells with an anti-LRIG1 antibody or polypeptide for a time sufficient to promote proliferation or inhibition of B cells and NK cells in the plurality of cells. In some embodiments, disclosed herein, are methods of promoting or inhibiting B cell or Natural Killer (NK) cell proliferation, comprising contacting a plurality of cells comprising one or more cells selected from a group consisting of B cells, NK cells, LRIG1-expressing cells, and VISTA-expressing cells with an anti-LRIG1 antibody or polypeptide for a time sufficient to promote proliferation or inhibition of B cells or NK cells in the plurality of cells. In some embodiments, disclosed herein, are methods of promoting or inhibiting B cell and Natural Killer (NK) cell proliferation, comprising contacting a plurality of cells comprising one or more cells selected from a group consisting of B cells, NK cells, LRIG1-expressing cells, and VISTA-expressing cells with an anti-LRIG1 antibody or polypeptide for a time sufficient to promote proliferation or inhibition of B cells and NK cells in the plurality of cells. In some cases, anti-LRIG1 antibody or polypeptide binds to LRIG1 and disrupts an interaction between LRIG1 and VISTA. In some cases, anti-LRIG1 antibody or polypeptide binds to LRIG1 and inhibits an interaction between LRIG1 and VISTA. In some embodiments, the anti-LRIG1 antibody or polypeptide binds to LRIG1 and enhances an interaction between LRIG1 and VISTA. In some embodiments, the anti-LRIG1 antibody or polypeptide is any one of the anti-LRIG1 antibodies or polypeptides disclosed herein. In some embodiments, the cells are Treg cells.

In some instances, the LRIG1-expressing cell disclosed herein is a tumor cell or an immune cell, or both. In some cases, the immune cell comprises immature myeloid cells, macrophages, dendritic cells, and IFNγ-producing Th1 cells. In some cases, LRIG1 is expressed in a plurality of cells located within a tumor microenvironment (TME). In some cases, the anti-LRIG1 antibody or antigen binding polypeptide induces a decrease of tumor cells within the TME. In some cases, the anti-LRIG1 antibody or polypeptide induces a decrease of tumor cells by at least or about 5%, 10%, 15%, 20%, 25%, 30%, 35%, 40%, 45%, 50%, 60%, 70%, 80%, or 90%, or any decrease within a range defined by any two of the aforementioned percentages. In some cases, the anti-LRIG1 antibody or polypeptide induces a decrease of tumor cells in a range of about 5% to about 95%, about 10% to about 90%, about 15% to about 80%, about 20% to about 70%, or about 30% to about 60%, or any decrease within a range defined by any two of the aforementioned percentages. In some cases, the anti-LRIG1 antibody or polypeptide induces a decrease of tumor cells by at least 30%.

In some instances, any one of the plurality of cells disclosed herein further comprises tumor-infiltrating lymphocytes (TILs). In some cases, the plurality of cells further comprises CD3+ T lymphocytes, CD4+ T helper cells, CD8+ cytotoxic T cells, or a combination thereof. In some cases, the plurality of cells further comprises CD3+ T lymphocytes. In some cases, the plurality of cells further comprises CD4+ T helper cells. In some cases, the plurality of cells further comprises CD8+ cytotoxic T cells. In some cases, the plurality of cells further comprises CD3+ T lymphocytes and CD4+ T helper cells. In some cases, the plurality of cells further comprises CD3+ T lymphocytes and CD8+ cytotoxic T cells. In some cases, the plurality of cells further comprises CD4+ T helper cells, CD8+ cytotoxic T cells. In some cases, the plurality of cells further comprises CD3+ T lymphocytes, CD4+ T helper cells, and CD8+ cytotoxic T cells.

In some instances, the contacting step of any one of the methods disclosed herein further induces TIL proliferation or inhibition of proliferation. In some cases, the contacting further induces or inhibits proliferation of CD3+ T lymphocytes, CD4+ T helper cells, CD8+ cytotoxic T cells, or a combination thereof. In some cases, the contacting further induces or inhibits proliferation of CD3+ T lymphocytes. In some cases, the contacting further induces or inhibits proliferation of CD4+ T helper cells. In some cases, the contacting further induces or inhibits proliferation of CD8+ cytotoxic T cells. In some cases, the contacting further induces or inhibits proliferation of CD3+ T lymphocytes and CD4+ T helper cells. In some cases, the contacting further induces or inhibits proliferation of CD3+ T lymphocytes and CD8+ cytotoxic T cells. In some cases, the contacting further induces or inhibits proliferation of CD4+ T helper cells and CD8+ cytotoxic T cells. In some cases, the contacting further induces or inhibits proliferation of CD3+ T lymphocytes, CD4+ T helper cells, and CD8+ cytotoxic T cells.

In some instances, the contacting step of any one of the methods disclosed herein further comprises an increase or decrease in proliferation of M1 macrophages. In some instances, the contacting further comprises an increase or decrease in M2 macrophage population within the TME. In some instances, the contacting further comprises an increase or decrease in proliferation of M1 macrophages and an increase or decrease in M2 macrophage population within the TME.

In some instances, the anti-LRIG1 antibody or antigen binding polypeptide binds to at least one amino acid residue within a LRIG1 region that corresponds to residues 245-260 of SEQ ID NO: 2. In some cases, the anti-LRIG1 antibody or polypeptide binds to at least one amino acid residue within a LRIG1 region that corresponds to residue 245 of SEQ ID NO: 2. In some cases, the anti-LRIG1 antibody or polypeptide binds to at least one amino acid residue within a LRIG1 region that corresponds to residue 246 of SEQ ID NO: 2. In some cases, the anti-LRIG1 antibody or polypeptide binds to at least one amino acid residue within a LRIG1 region that corresponds to residue 247 of SEQ ID NO: 2. In some cases, the anti-LRIG1 antibody or polypeptide binds to at least one amino acid residue within a LRIG1 region that corresponds to residue 248 of SEQ ID NO: 2. In some cases, the anti-LRIG1 antibody or polypeptide binds to at least one amino acid residue within a LRIG1 region that corresponds to residue 249 of SEQ ID NO: 2. In some cases, the anti-LRIG1 antibody or polypeptide binds to at least one amino acid residue within a LRIG1 region that corresponds to residue 250 of SEQ ID NO: 2. In some cases, the anti-LRIG1 antibody or polypeptide binds to at least one amino acid residue within a LRIG1 region that corresponds to residue 251 of SEQ ID NO: 2. In some cases, the anti-LRIG1 antibody or polypeptide binds to at least one amino acid residue within a LRIG1 region that corresponds to residue 252 of SEQ ID NO: 2. In some cases, the anti-LRIG1 antibody or polypeptide binds to at least one amino acid residue within a LRIG1 region that corresponds to residue 253 of SEQ ID NO: 2. In some cases, the anti-LRIG1 antibody or polypeptide binds to at least one amino acid residue within a LRIG1 region that corresponds to residue 254 of SEQ ID NO: 2. In some cases, the anti-LRIG1 antibody or polypeptide binds to at least one amino acid residue within a LRIG1 region that corresponds to residue 255 of SEQ ID NO: 2. In some cases, the anti-LRIG1 antibody or polypeptide binds to at least one amino acid residue within a LRIG1 region that corresponds to residue 256 of SEQ ID NO: 2. In some cases, the anti-LRIG1 antibody or polypeptide binds to at least one amino acid residue within a LRIG1 region that corresponds to residue 257 of SEQ ID NO: 2. In some cases, the anti-LRIG1 antibody or polypeptide binds to at least one amino acid residue within a LRIG1 region that corresponds to residue 258 of SEQ ID NO: 2. In some cases, the anti-LRIG1 antibody or polypeptide binds to at least one amino acid residue within a LRIG1 region that corresponds to residue 259 of SEQ ID NO: 2. In some cases, the anti-LRIG1 antibody or polypeptide binds to at least one amino acid residue within a LRIG1 region that corresponds to residue 260 of SEQ ID NO: 2. In some embodiments, the anti-LRIG1 antibody or polypeptide is any one of the anti-LRIG1 antibodies or polypeptides disclosed herein.

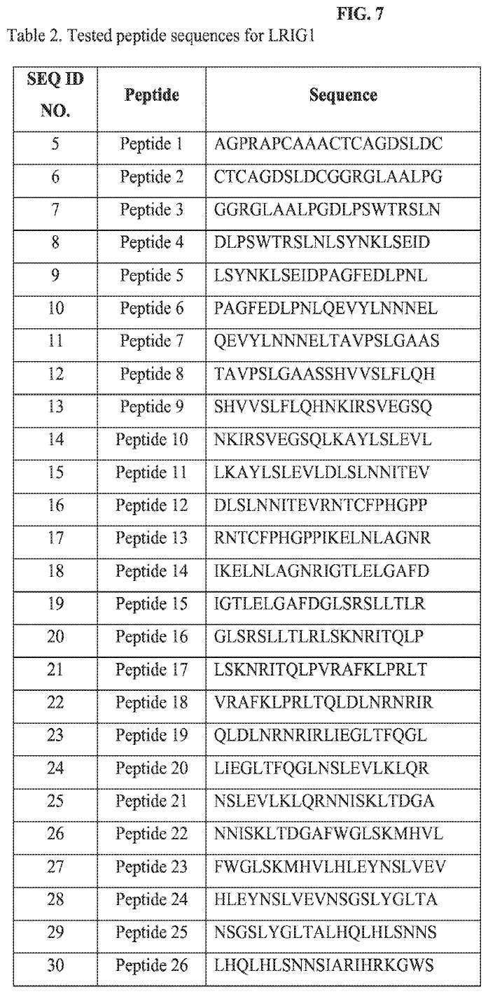

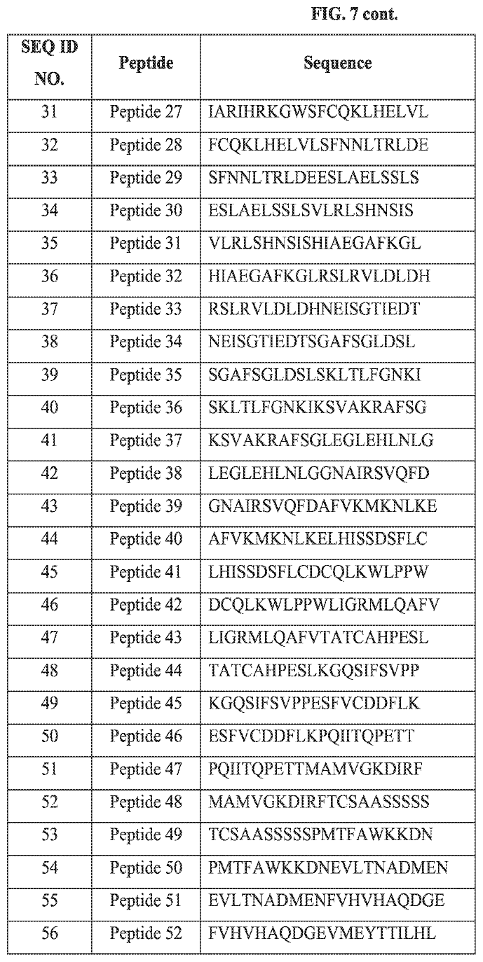

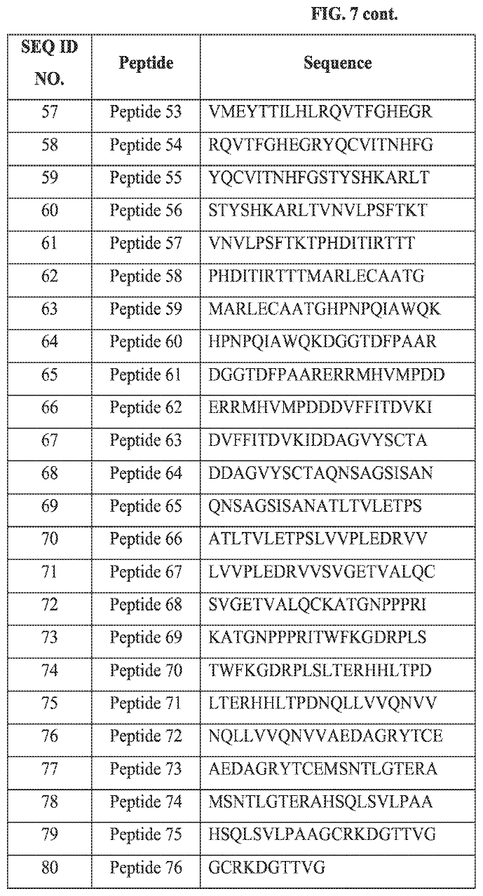

Peptide sequences for regions of LRIG1 are listed in FIG. 7.

In some cases, the anti-LRIG1 antibody or antigen binding polypeptide disclosed anywhere herein binds to at least one amino acid residue within Peptide 1, Peptide 2, Peptide 3, Peptide 4, Peptide 5, Peptide 6, Peptide 7, Peptide 8, Peptide 9, Peptide 10, Peptide 11, Peptide 12, Peptide 13, Peptide 14, Peptide 15, Peptide 16, Peptide 17, Peptide 18, Peptide 19, Peptide 20, Peptide 21, Peptide 22, Peptide 23, Peptide 24, Peptide 25, Peptide 26, Peptide 27, Peptide 28, Peptide 29, Peptide 30, Peptide 31, Peptide 32, Peptide 33, Peptide 34, Peptide 35, Peptide 36, Peptide 37, Peptide 38, Peptide 39, Peptide 40, Peptide 41, Peptide 42, Peptide 43, Peptide 44, Peptide 45, Peptide 46, Peptide 47, Peptide 48, Peptide 49, Peptide 50, Peptide 51, Peptide 52, Peptide 53, Peptide 55, Peptide 56, Peptide 57, Peptide 58, Peptide 59, Peptide 60, Peptide 62, Peptide 63, Peptide 64, Peptide 65, Peptide 66, Peptide 67, Peptide 68, Peptide 69, Peptide 70, Peptide 71, Peptide 72, Peptide 73, Peptide 74, Peptide 75, or Peptide 76. In some cases, the anti-LRIG1 antibody or polypeptide binds to at least one amino acid residue within Peptide 54. In some cases, the anti-LRIG1 antibody or polypeptide binds to at least one amino acid residue within Peptide 61.

In some cases, the anti-LRIG1 antibody or antigen binding polypeptide disclosed anywhere herein binds to at least one amino acid residue within a peptide, wherein the peptide has a sequence as set forth in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 62, 63, 64, 65, 66, 67, 68, 69, 70, 71, 72, 73, 74, 75, 76, 77, 78, 79, or 80.

In some instances, the anti-LRIG1 antibody or antigen binding polypeptide disclosed anywhere herein comprises a binding affinity (e.g., k_{D}) to LRIG1 of less than 1 nM, less than 1.2 nM, less than 2 nM, less than 5 nM, less than 10 nM, less than 13.5 nM, less than 15 nM, less than 20 nM, less than 25 nM, or less than 30 nM. In some instances, the anti-LRIG1 antibody or polypeptide comprises a k_{D} of less than 1 nM. In some instances, the anti-LRIG1 antibody or polypeptide comprises a k_{D} of less than 1.2 nM. In some instances, the anti-LRIG1 antibody comprises a k_{D} of less than 2 nM. In some instances, the anti-LRIG1 antibody or polypeptide comprises a k_{D} of less than 5 nM. In some instances, the anti-LRIG1 antibody or polypeptide comprises a k_{D} of less than 10 nM. In some instances, the anti-LRIG1 antibody or polypeptide comprises a k_{D} of less than 13.5 nM. In some instances, the anti-LRIG1 antibody or polypeptide comprises a k_{D} of less than 15 nM. In some instances, the anti-LRIG1 antibody or polypeptide comprises a k_{D} of less than 20 nM. In some instances, the anti-LRIG1 antibody or polypeptide comprises a k_{D} of less than 25 nM. In some instances, the anti-LRIG1 antibody or polypeptide comprises a k_{D} of less than 30 nM.

In some instances, the anti-LRIG1 antibody or antigen binding polypeptide disclosed anywhere herein comprises a humanized antibody. In other instances, the anti-LRIG1 antibody or polypeptide comprises a chimeric antibody. In some cases, the anti-LRIG1 antibody or polypeptide comprises a full-length antibody or polypeptide or a binding fragment thereof. In some cases, the anti-LRIG1 antibody or polypeptide comprises a bispecific antibody or a binding fragment thereof. In some cases, the anti-LRIG1 antibody or polypeptide comprises a monovalent Fab′, a divalent Fab2, a single-chain variable fragment (scFv), a diabody, a minibody, a nanobody, a single-domain antibody (sdAb), or a camelid antibody or binding fragment thereof.

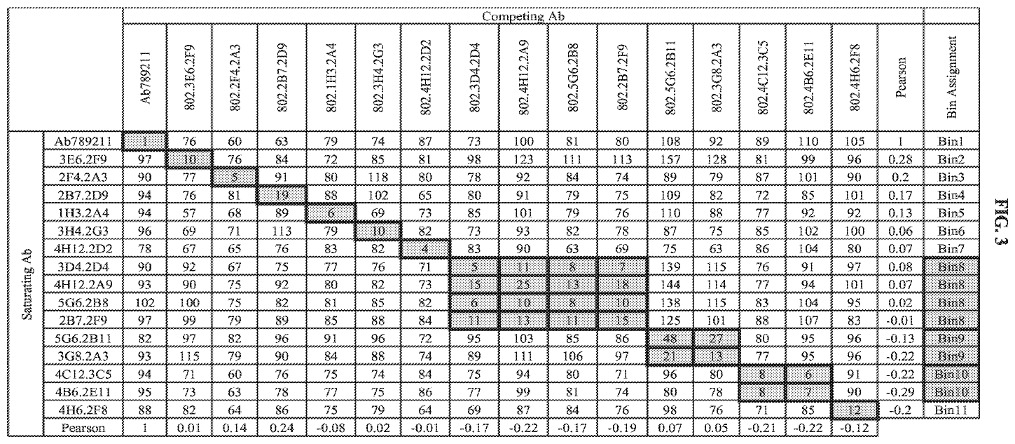

In some instances, the anti-LRIG1 antibody or antigen binding polypeptide disclosed anywhere herein is a bispecific antibody or binding fragment thereof. Exemplary bispecific antibody formats include, but are not limited to, Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), Azymetric, Bispecific Engagement by Antibodies based on the T-cell receptor (BEAT), Bispecific T-cell Engager (BiTE), Biclonics, Fab-scFv-Fc, Two-in-one/Dual Action Fab (DAF), FinomAb, scFv-Fc-(Fab)-fusion, Dock-aNd-Lock (DNL), Adaptir (previously SCORPION), Tandem diAbody (TandAb), Dual-affinity-ReTargeting (DART), nanobody, triplebody, tandems scFv (taFv), triple heads, tandem dAb/VHH, triple dAb/VHH, or tetravalent dAb/VHH. In some cases, the anti-VISTA antibody, the anti-LRIG1 antibody, or combination thereof is a bispecific antibody or binding fragment thereof comprising a bispecific antibody format illustrated in FIG. 2 of Brinkmann and Kontermann, “The making of bispecific antibodies,” MABS 9(2): 182-212 (2017).

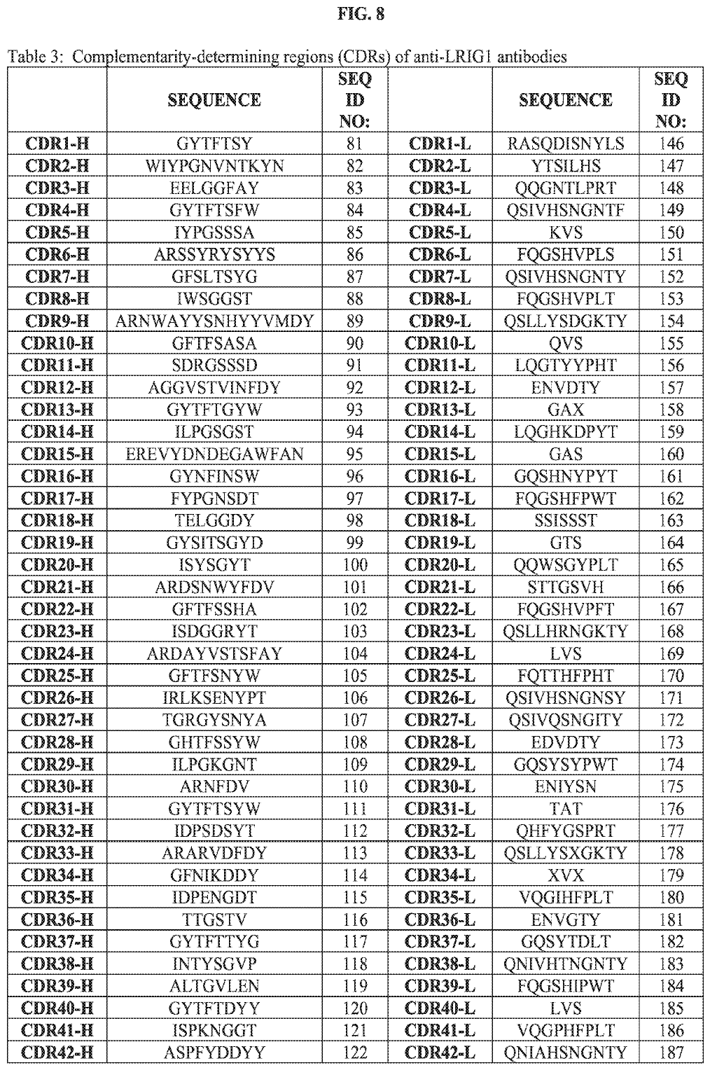

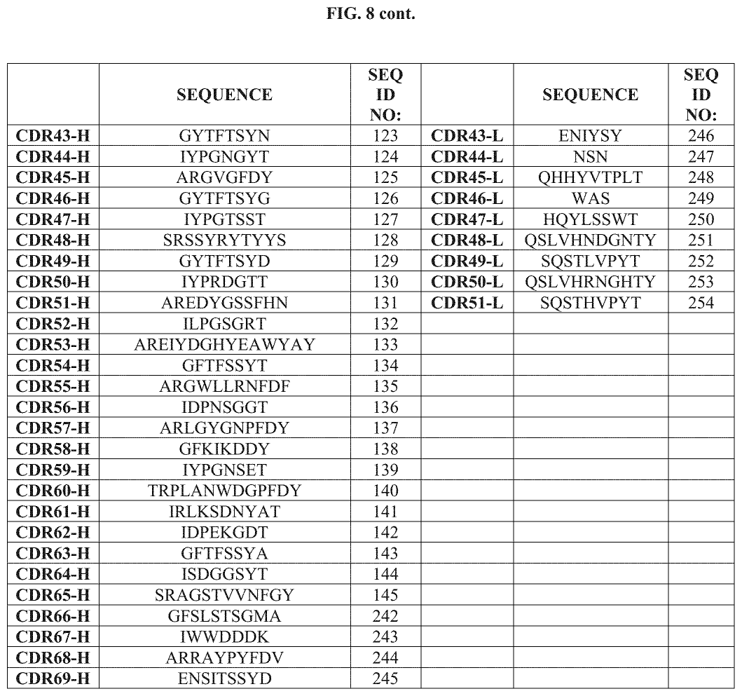

In some embodiments, the anti-LRIG1 antibody or antigen binding polypeptide disclosed anywhere herein is a humanized antibody comprising the complementarity-determining regions (CDRs) illustrated in FIG. 8.

In some cases, the humanized anti-LRIG1 antibody comprises a heavy chain variable region (VH) and a light chain variable region (VL). In some cases, the VH or VL regions of the humanized anti-LRIG1 antibody may comprise at least one, at least two, or three CDRs. In some embodiments, the antibody comprises at least one heavy chain CDR1 selected from the group consisting of 84, 87, 90, 93, 96, 99, 102, 105, 108, 111, 114, 117, 120, 123, 126, 129, 134, 138, 143, and 242. In some embodiments, the antibody comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243. In some embodiments, the antibody comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244. In some embodiments, the antibody comprises at least one light chain CDR1 selected from the group consisting of SEQ ID NOs: 149, 152, 154, 157, 163, 166, 168, 171, 172, 173, 175, 178, 181, 183, 187, 246, 251, and 253. In some embodiments, the antibody comprises at least one light chain CDR2 selected from the group consisting of SEQ ID NOs: 150, 155, 158, 160, 164, 169, 176, 179, 185, 247, and 249. In some embodiments, the antibody comprises at least one light chain CDR3 selected from the group consisting of SEQ ID NOs: 151, 153, 156, 159, 161, 162, 165, 167, 170, 174, 177, 180, 182, 184, 186, 248, 250, and 252. In some embodiments, the antibody comprises at least one heavy chain CDR selected from SEQ ID NOs: 84-145 or 242-245. In some embodiments, the antibody comprises at least one light chain CDR selected from SEQ ID NOs: 147-187 or 246-254. In some cases, the humanized anti-LRIG1 antibody comprises CDRs within the VH and VL sequences as illustrated in FIG. 9. In some embodiments, the antibody comprises 3 heavy chain CDRs and 3 light chain CDRs according to FIG. 9.

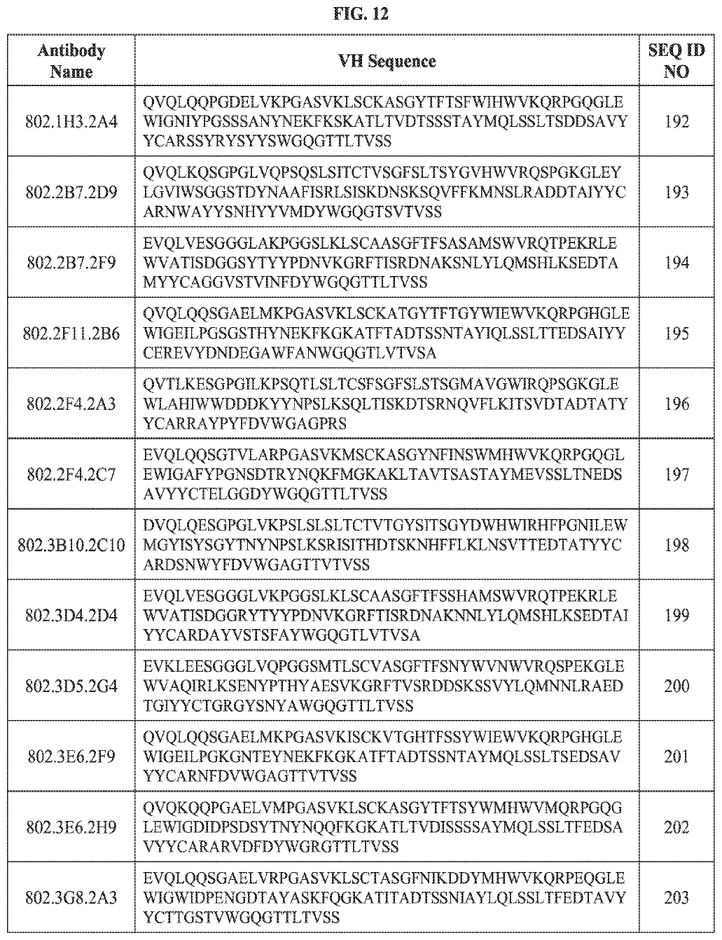

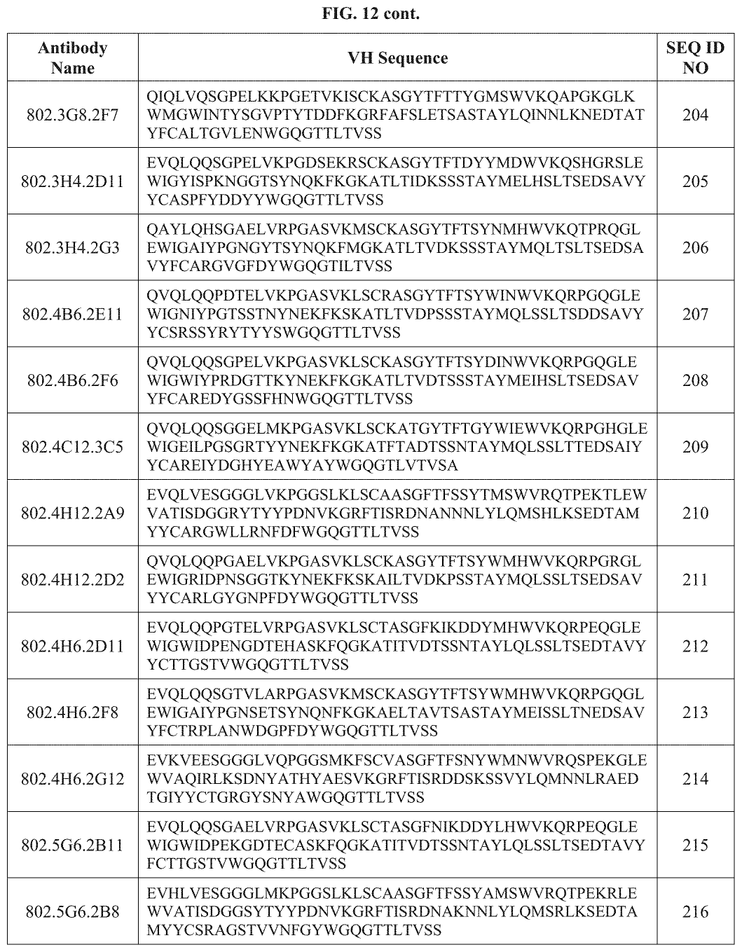

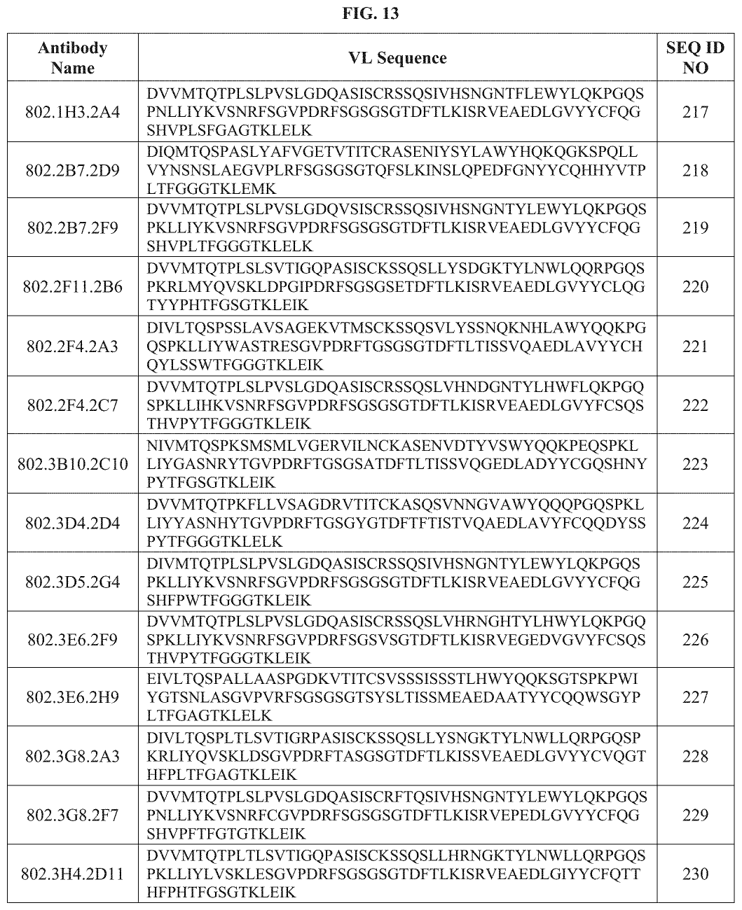

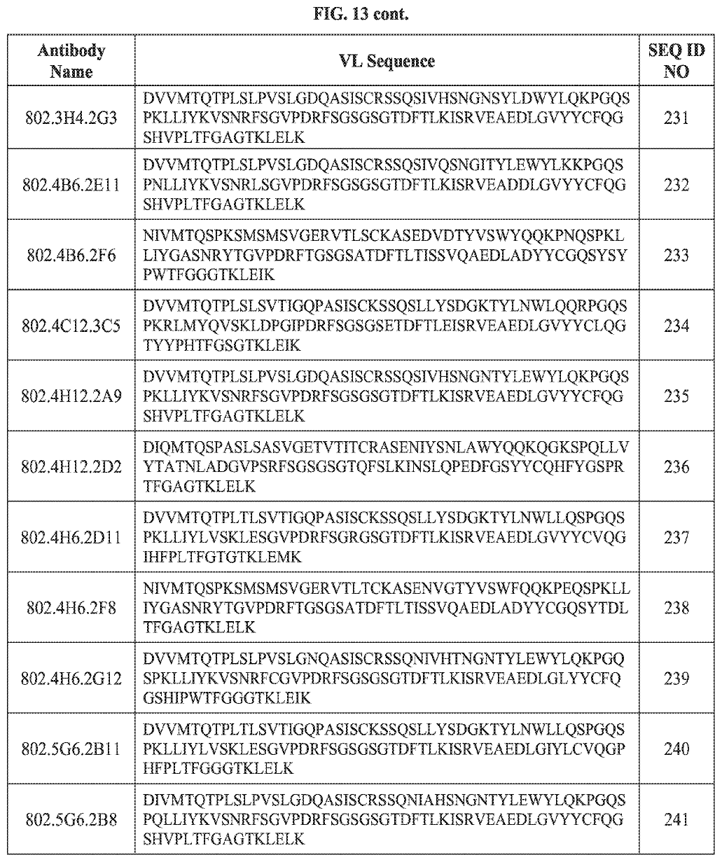

In some cases, the humanized anti-LRIG1 antibody comprises a heavy chain variable region (VH) and/or a light chain variable region (VL). In some cases, the humanized anti-LRIG1 antibody comprises a VH sequence as illustrated in FIG. 12. In some cases, the humanized anti-LRIG1 antibody comprises a VL sequence as illustrated in FIG. 13. In some embodiments, the antibody or antigen binding polypeptide comprises a VH domain having a sequence with at least 80%, 85%, 90%, 95%, 96%, 97%, 98%, 99%, or 100% homology to SEQ ID NOs: 192-216. In some embodiments, the antibody or antigen binding polypeptide comprises a VL domain having a sequence with at least 80%, 85%, 90%, 95%, 96%, 97%, 98%, 99%, or 100% homology to SEQ ID NOs: 217-241. In some embodiments, the humanized anti-LRIG1 antibody comprises a VH sequence and VL sequence as disclosed in Table 5.

TABLE 5
Antibodies and associated VH and VL sequences

| Antibody | VH sequence | VL sequence |
|---|---|---|
| 802.1H3.2A4 | SEQ ID NO: 192 | SEQ ID NO: 217 |
| 802.2B7.2D9 | SEQ ID NO: 193 | SEQ ID NO: 218 |
| 802.2B7.2F9 | SEQ ID NO: 194 | SEQ ID NO: 219 |
| 802.2F11.2B6 | SEQ ID NO: 195 | SEQ ID NO: 220 |
| 802.2F4.2A3 | SEQ ID NO: 196 | SEQ ID NO: 221 |
| 802.2F4.2C7 | SEQ ID NO: 197 | SEQ ID NO: 222 |
| 802.3B10.2C10 | SEQ ID NO: 198 | SEQ ID NO: 223 |
| 802.3D4.2D4 | SEQ ID NO: 199 | SEQ ID NO: 224 |
| 802.3D5.2G4 | SEQ ID NO: 200 | SEQ ID NO: 225 |
| 802.3E6.2F9 | SEQ ID NO: 201 | SEQ ID NO: 226 |
| 802.3E6.2H9 | SEQ ID NO: 202 | SEQ ID NO: 227 |
| 802.3G8.2A3 | SEQ ID NO: 203 | SEQ ID NO: 228 |
| 802.3G8.2F7 | SEQ ID NO: 204 | SEQ ID NO: 229 |
| 802.3H4.2D11 | SEQ ID NO: 205 | SEQ ID NO: 230 |
| 802.3H4.2G3 | SEQ ID NO: 206 | SEQ ID NO: 231 |
| 802.4B6.2E11 | SEQ ID NO: 207 | SEQ ID NO: 232 |
| 802.4B6.2F6 | SEQ ID NO: 208 | SEQ ID NO: 233 |
| 802.4C12.3C5 | SEQ ID NO: 209 | SEQ ID NO: 234 |
| 802.4H12.2A9 | SEQ ID NO: 210 | SEQ ID NO: 235 |
| 802.4H12.2D2 | SEQ ID NO: 211 | SEQ ID NO: 236 |
| 802.4H6.2D11 | SEQ ID NO: 212 | SEQ ID NO: 237 |
| 802.4H6.2F8 | SEQ ID NO: 213 | SEQ ID NO: 238 |
| 802.4H6.2G12 | SEQ ID NO: 214 | SEQ ID NO: 239 |
| 802.5G6.2B11 | SEQ ID NO: 215 | SEQ ID NO: 240 |
| 802.5G6.2B8 | SEQ ID NO: 216 | SEQ ID NO: 241 |

In some cases, the humanized anti-LRIG1 antibody is 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, or 802.5G6.2B8, or any combination thereof.

In some embodiments, the anti-LRIG1 antibody or antigen binding polypeptide comprises a framework region selected from IgM, IgG (e.g., IgG1, IgG2, IgG3, or IgG4), IgA, IgD, or IgE. In some cases, the anti-LRIG1 antibody or polypeptide comprises an IgM framework. In some cases, the anti-LRIG1 antibody or polypeptide comprises an IgG (e.g., IgG1, IgG2, IgG3, or IgG4) framework. In some cases, the anti-LRIG1 antibody or polypeptide comprises an IgG1 framework. In some cases, the anti-LRIG1 antibody or polypeptide comprises an IgG2 framework. In some cases, the anti-LRIG1 antibody or polypeptide comprises an IgG4 framework.

In some embodiments, the anti-LRIG1 antibody or polypeptide comprises one or more mutations in the framework region, e.g., in the CH1 domain, CH2 domain, CH3 domain, hinge region, or a combination thereof. In some cases, the one or more mutations modulate Fc receptor interactions, e.g., to increase Fc effector functions such as ADCC and/or complement-dependent cytotoxicity (CDC). In some cases, the one or more mutations stabilize the antibody or polypeptide and/or increase the half-life of the antibody or polypeptide. In additional cases, the one or more mutations modulate glycosylation.

### Method of Treatment

In some embodiments, also disclosed herein is a method of administering to a subject in need thereof an anti-LRIG1 antibody or antigen binding polypeptide described supra. In some embodiments, the subject in need thereof has a disease, has previously had a disease, or is at risk of contracting a disease. In some embodiments, the disease is cancer. In some embodiments, the disease is an immune-related disorder or condition. In some embodiments, the immune-related disorder or condition is an autoimmune or inflammatory disease.

In some instances, the subject is diagnosed with a cancer. In some cases, the cancer is a solid tumor. In other instances, the cancer is a hematologic malignancy. In additional instances, the cancer is a metastatic, a relapsed, or a refractory cancer.

In some instances, the cancer is a solid tumor. In some cases, the cancer is breast cancer, colorectal cancer, kidney cancer, liver cancer, lung cancer, brain cancer, pancreatic cancer, bladder cancer, or stomach cancer, or any combination thereof. In some cases, the lung cancer comprises a non-small cell lung cancer (NSCLC) such as lung adenocarcinoma, squamous cell carcinoma, or large cell carcinoma; or small cell lung cancer (SCLC).

In some cases, the cancer is a hematologic malignancy, e.g., a metastatic, relapsed, or refractory hematologic malignancy.

In some embodiments, the subject has an immune-related disorder or condition. In some embodiments, the immune-related disorder or condition comprises an autoimmune disease, an inflammatory disease, or wound healing. In some embodiments, the immune-related disorder includes but is not limited to fibrosis, liver fibrosis, kidney fibrosis, cardiac fibrosis, pulmonary fibrosis, non-alcoholic fatty liver disease, non-alcoholic steatohepatitis, sepsis, atopic dermatitis, psoriasis, systemic lupus erythematosus, inflammatory bowel syndrome, arthritis, or any combination thereof.

In some instances, the anti-LRIG1 antibody or antigen binding polypeptide is formulated for systemic administration. In some instances, the anti-LRIG1 antibody or antigen binding polypeptide is formulated for parenteral administration.

In some embodiments, the anti-LRIG1 antibody or antigen binding polypeptide is administered to the subject in combination with an additional therapeutic agent. In some instances, the additional therapeutic agent comprises an immunotherapeutic agent. In some instances, the additional therapeutic agent comprises an immune checkpoint modulator. In some instances, the additional therapeutic agent comprises a chemotherapeutic agent, targeted therapeutic agent, hormonal therapeutic agent, or a stem cell-based therapeutic agent.

In some instances, the additional therapeutic agent comprises an immunotherapeutic agent. In some embodiments, the immunotherapeutic agent is used for an immunotherapy. In some instances, the immunotherapy is an adoptive cell therapy. Exemplary adoptive cell therapies include AFP TCR, MAGE-A10 TCR, or NY-ESO-TCR from Adaptimmune; ACTR087/rituximab from Unum Therapeutics; anti-BCMA CAR-T cell therapy, anti-CD19 “armored” CAR-T cell therapy, JCAR014, JCAR018, JCAR020, JCAR023, JCAR024, or JTCR016 from Juno Therapeutics; JCAR017 from Celgene/Juno Therapeutics; anti-CD19 CAR-T cell therapy from Intrexon; anti-CD19 CAR-T cell therapy, axicabtagene ciloleucel, KITE-718, KITE-439, or NY-ESO-1 T-cell receptor therapy from Kite Pharma; anti-CEA CAR-T therapy from Sorrento Therapeutics; anti-PSMA CAR-T cell therapy from TNK Therapeutics/Sorrento Therapeutics; ATA520 from Atara Biotherapeutics; AU101 and AU105 from Aurora BioPharma; baltaleucel-T (CMD-003) from Cell Medica; bb2121 from bluebird bio; BPX-501, BPX-601, or BPX-701 from Bellicum Pharmaceuticals; BSK01 from Kiromic; IMCgp100 from Immunocore; JTX-2011 from Jounce Therapeutics; LN-144 or LN-145 from Lion Biotechnologies; MB-101 or MB-102 from Mustang Bio; NKR-2 from Celyad; PNK-007 from Celgene; tisagenlecleucel-T from Novartis Pharmaceuticals; or TT12 from Tessa Therapeutics.

In some instances, the immunotherapy is a dendritic cell-based therapy.

In some instances, the immunotherapy comprises a cytokine-based therapy, comprising e.g., an interleukin (IL) such as IL-2, IL-15, or IL-21, interferon (IFN)-α, or granulocyte macrophage colony-stimulating factor (GM-CSF).

In some instances, the immunotherapy comprises an immune checkpoint modulator. Exemplary immune checkpoint modulators include PD-1 modulators such as nivolumab (Opdivo) from Bristol-Myers Squibb, pembrolizumab (Keytruda) from Merck, AGEN 2034 from Agenus, BGB-A317 from BeiGene, B1-754091 from Boehringer-Ingelheim Pharmaceuticals, CBT-501 (genolimzumab) from CBT Pharmaceuticals, INCSHR1210 from Incyte, JNJ-63723283 from Janssen Research & Development, MEDI0680 from MedImmune, MGA 012 from MacroGenics, PDR001 from Novartis Pharmaceuticals, PF-06801591 from Pfizer, REGN2810 (SAR439684) from Regeneron Pharmaceuticals/Sanofi, or TSR-042 from TESARO; CTLA-4 modulators such as ipilimumab (Yervoy), or AGEN 1884 from Agenus; PD-L1 modulators such as durvalumab (Imfinzi) from AstraZeneca, atezolizumab (MPDL3280A) from Genentech, avelumab from EMD Serono/Pfizer, CX-072 from CytomX Therapeutics, FAZ053 from Novartis Pharmaceuticals, KNO35 from 3D Medicine/Alphamab, LY3300054 from Eli Lilly, or M7824 (anti-PD-L1/TGFbeta trap) from EMD Serono; LAGS modulators such as BMS-986016 from Bristol-Myers Squibb, IMP701 from Novartis Pharmaceuticals, LAG525 from Novartis Pharmaceuticals, or REGN3767 from Regeneron Pharmaceuticals; OX40 modulators such as BMS-986178 from Bristol-Myers Squibb, GSK3174998 from GlaxoSmithKline, INCAGN1949 from Agenus/Incyte, MEDI0562 from MedImmune, PF-04518600 from Pfizer, or RG7888 from Genentech; GITR modulators such as GWN323 from Novartis Pharmaceuticals, INCAGN1876 from Agenus/Incyte, MEDI1873 from MedImmune, MK-4166 from Merck, or TRX518 from Leap Therapeutics; KIR modulators such as lirilumab from Bristol-Myers Squibb; or TIM modulators such as MBG453 from Novartis Pharmaceuticals or TSR-022 from Tesaro.

In some instances, the additional therapeutic agent comprises a chemotherapeutic agent. Exemplary chemotherapeutic agents include, but are not limited to, alkylating agents such as cyclophosphamide, mechlorethamine, chlorambucil, melphalan, dacarbazine, or nitrosoureas; anthracyclines such as daunorubicin, doxorubicin, epirubicin, idarubicin, mitoxantrone, or valrubicin; cytoskeletal disruptors such as paclitaxel, docetaxel, abraxane, or taxotere; epothilones; histone deacetylase inhibitors such as vorinostat or romidepsin; topoisomerase I inhibitors such as irinotecan or topotecan; topoisomerase II inhibitors such as etoposide, teniposide, or tafluposide; kinase inhibitors such as bortezomib, erlotinib, gefitinib, imatinib, vemurafenib, or vismodegib; nucleotide analogs and precursor analogs such as azacitidine, azathioprine, capecitabine, cytarabine, doxifluridine, fluorouracil, gemcitabine, hydrozyurea, mercaptopurine, methotrexate, or tioguanine; platinum-based agents such as carboplatin, cisplatin, or oxaliplatin; retinoids such as tretinoin, alitretinoin, or bexarotene; or vinca alkaloids and derivatives such as vinblastine, vincristine, vindesine, or vinorelbine.

In some instances, the additional therapeutic agent comprises a hormone-based therapeutic agent. Exemplary hormone-based therapeutic agents include, but are not limited to, aromatase inhibitors such as letrozole, anastrozole, exemestane, or aminoglutethimide; gonadotropin-releasing hormone (GnRH) analogues such as leuprorelin or goserelin; selective estrogen receptor modulators (SERMs) such as tamoxifen, raloxifene, toremifene, or fulvestrant; antiandrogens such as flutamide or bicalutamide; progestogens such as megestrol acetate or medroxyprogesterone acetate; androgens such as fluoxymesterone; estrogens such as estrogen diethylstilbestrol (DES), Estrace, or polyestradiol phosphate; or somatostatin analogs such as octreotide.

In some instances, the additional therapeutic agent is a first-line therapeutic agent.

In some embodiments, the anti-LRIG1 antibody or antigen binding polypeptide and the additional therapeutic agent are administered simultaneously. In some instances, the anti-LRIG1 antibody or antigen binding polypeptide and the additional therapeutic agent are administered sequentially. In such instances, the anti-LRIG1 antibody or antigen binding polypeptide is administered to the subject prior to administering the additional therapeutic agent. In other instances, the anti-LRIG1 antibody or antigen binding polypeptide is administered to the subject after the additional therapeutic agent is administered.

In some cases, the additional therapeutic agent and the anti-LRIG1 antibody or antigen binding polypeptide are formulated as separate dosage.

In some instances, the subject has undergone surgery. In some cases, the anti-LRIG1 antibody or antigen binding polypeptide and optionally the additional therapeutic agent are administered to the subject prior to surgery. In some instances, the anti-LRIG1 antibody or antigen binding polypeptide and optionally the additional therapeutic agent are administered to the subject after surgery.

In some instances, the subject has undergone radiation. In some instances, the anti-LRIG1 antibody or antigen binding polypeptide and optionally the additional therapeutic agent are administered to the subject during or after radiation treatment. In some cases, the anti-LRIG1 antibody or antigen binding polypeptide and optionally the additional therapeutic agent are administered to the subject prior to undergoing radiation.

In some embodiments, the subject is a mammal. In some instances, the subject is a human.

### Antibody Production

In some embodiments, anti-LRIG1 antibodies are raised by standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.). When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Polyclonal or monoclonal anti-LRIG1 antibodies can be produced from animals which have been genetically altered to produce human immunoglobulins. A transgenic animal can be produced by initially producing a “knock-out” animal which does not produce the animal's natural antibodies, and stably transforming the animal with a human antibody locus (e.g., by the use of a human artificial chromosome). In such cases, only human antibodies are then made by the animal. Techniques for generating such animals, and deriving antibodies therefrom, are described in U.S. Pat. Nos. 6,162,963 and 6,150,584, incorporated fully herein by reference. Such antibodies can be referred to as human xenogenic antibodies.

Alternatively, anti-LRIG1 antibodies can be produced from phage libraries containing human variable regions. See U.S. Pat. No. 6,174,708, incorporated fully herein by reference.

In some aspects of any of the embodiments disclosed herein, an anti-LRIG1 antibody is produced by a hybridoma.

For monoclonal anti-LRIG1 antibodies, hybridomas may be formed by isolating the stimulated immune cells, such as those from the spleen of the inoculated animal. These cells can then be fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

In addition, the anti-LRIG1 antibody may be produced by genetic engineering.

Anti-LRIG1 antibodies disclosed herein can have a reduced propensity to induce an undesired immune response in humans, for example, anaphylactic shock, and can also exhibit a reduced propensity for priming an immune response which would prevent repeated dosage with an antibody therapeutic or imaging agent (e.g., the human-anti-murine-antibody “HAMA” response). Such anti-LRIG1 antibodies include, but are not limited to, humanized, chimeric, or xenogenic human anti-LRIG1 antibodies.

Chimeric anti-LRIG1 antibodies can be made, for example, by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference).

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In some examples, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Humanized antibodies can be engineered to contain human-like immunoglobulin domains, and incorporate only the complementarity-determining regions of the animal-derived antibody. This can be accomplished by carefully examining the sequence of the hyper-variable loops of the variable regions of a monoclonal antigen binding unit or monoclonal antibody, and fitting them to the structure of a human antigen binding unit or human antibody chains. See, e.g., U.S. Pat. No. 6,187,287, incorporated fully herein by reference.

Methods for humanizing non-human antibodies are well known in the art. “Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. See, for example, EP 0329400. In some versions, V regions are humanized by designing consensus sequences of human and mouse V regions, and converting residues outside the CDRs that are different between the consensus sequences.

In principle, a framework sequence from a humanized antibody can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a human antibody (HuAb) is to the original murine antibody (muAb), the less likely that the human framework will introduce distortions into the murine CDRs that could reduce affinity. Based on a sequence homology search against an antibody sequence database, the HuAb IC4 provides good framework homology to muM4TS 0.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The invention thus encompasses HuAbs with different variable (V) regions. It is within the skill of one in the art to determine suitable V region sequences and to optimize these sequences. Methods for obtaining antibodies with reduced immunogenicity are also described in U.S. Pat. No. 5,270,202 and EP 699,755.

Humanized antibodies can be prepared by a process of analysis of the parental sequences and various conceptual humanized products using three dimensional models of the parental and humanized sequences. Three-dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

A process for humanization of subject antigen binding units can be as follows. The best-fit germline acceptor heavy and light chain variable regions are selected based on homology, canonical structure and physical properties of the human antibody germlines for grafting. Computer modeling of mVH/VL versus grafted hVH/VL is performed and prototype humanized antibody sequence is generated. If modeling indicated a need for framework back-mutations, second variant with indicated FW changes is generated. DNA fragments encoding the selected germline frameworks and murine CDRs are synthesized. The synthesized DNA fragments are subcloned into IgG expression vectors and sequences are confirmed by DNA sequencing. The humanized antibodies are expressed in cells, such as 293F and the proteins are tested, for example in MDM phagocytosis assays and antigen binding assays. The humanized antigen binding units are compared with parental antigen binding units in antigen binding affinity, for example, by FACS on cells expressing the target antigen. If the affinity is greater than 2-fold lower than parental antigen binding unit, a second round of humanized variants can be generated and tested as described above.

As noted above, an anti-LRIG1 antibody can be either “monovalent” or “multivalent.” Whereas the former has one binding site per antigen-binding unit, the latter contains multiple binding sites capable of binding to more than one antigen of the same or different kind. Depending on the number of binding sites, antigen binding units may be bivalent (having two antigen-binding sites), trivalent (having three antigen-binding sites), tetravalent (having four antigen-binding sites), and so on.

Multivalent anti-LRIG1 antibodies can be further classified on the basis of their binding specificities. A “monospecific” anti-LRIG1 antibody is a molecule capable of binding to one or more antigens of the same kind. A “multispecific” anti-LRIG1 antibody is a molecule having binding specificities for at least two different antigens. While such molecules normally will only bind two distinct antigens (i.e. bispecific anti-LRIG1 antibodies), antibodies with additional specificities such as trispecific antibodies are encompassed by this expression when used herein. This disclosure further provides multispecific anti-LRIG1 antibodies. Multispecific anti-LRIG1 antibodies are multivalent molecules capable of binding to at least two distinct antigens, e.g., bispecific and trispecific molecules exhibiting binding specificities to two and three distinct antigens, respectively.

### Polynucleotides and Vectors

In some embodiments, the present disclosure provides isolated nucleic acids encoding any of the anti-LRIG1 antibodies disclosed herein. In another embodiment, the present disclosure provides vectors comprising a nucleic acid sequence encoding any anti-LRIG1 antibody or antigen binding polypeptide disclosed herein. In some embodiments, this invention provides isolated nucleic acids that encode a light-chain CDR and a heavy-chain CDR of an anti-LRIG1 antibody disclosed herein.

The subject anti-LRIG1 antibodies or antigen binding polypeptides can be prepared by recombinant DNA technology, synthetic chemistry techniques, or a combination thereof. For instance, sequences encoding the desired components of the anti-LRIG1 antibodies, including light chain CDRs and heavy chain CDRs are typically assembled cloned into an expression vector using standard molecular techniques know in the art. These sequences may be assembled from other vectors encoding the desired protein sequence, from PCR-generated fragments using respective template nucleic acids, or by assembly of synthetic oligonucleotides encoding the desired sequences. Expression systems can be created by transfecting a suitable cell with an expressing vector which comprises an anti-LRIG1 antibody of interest.

Nucleotide sequences corresponding to various regions of light or heavy chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection (atcc.org), which offers a diverse collection of well-characterized hybridoma cell lines. Alternatively, antibody nucleotides can be obtained from immunized or non-immunized rodents or humans, and form organs such as spleen and peripheral blood lymphocytes. Specific techniques applicable for extracting and synthesizing antibody nucleotides are described in Orlandi et al. (1989) Proc. Natl. Acad. Sci. U.S.A 86: 3833-3837; Larrick et al. (1989) Biochem. Biophys. Res. Commun. 160:1250-1255; Sastry et al. (1989) Proc. Natl. Acad. Sci., U.S.A. 86: 5728-5732; and U.S. Pat. No. 5,969,108.

Polynucleotides encoding anti-LRIG1 antibodies can also be modified, for example, by substituting the coding sequence for human heavy and light chain constant regions in place of the homologous non-human sequences. In that manner, chimeric antibodies are prepared that retain the binding specificity of the original anti-LRIG1 antibody.

### Host Cells

In some embodiments, the present disclosure provides host cells expressing any one of the anti-LRIG1 antibodies disclosed herein. A subject host cell typically comprises a nucleic acid encoding any one of the anti-LRIG1 antibodies disclosed herein.

The invention provides host cells transfected with the polynucleotides, vectors, or a library of the vectors described above. The vectors can be introduced into a suitable prokaryotic or eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

For most animal cells, any of the above-mentioned methods is suitable for vector delivery. Preferred animal cells are vertebrate cells, preferably mammalian cells, capable of expressing exogenously introduced gene products in large quantity, e.g. at the milligram level. Non-limiting examples of preferred cells are NIH3T3 cells, COS, HeLa, and CHO cells.

Once introduced into a suitable host cell, expression of the anti-LRIG1 antibodies can be determined using any nucleic acid or protein assay known in the art. For example, the presence of transcribed mRNA of light chain CDRs or heavy chain CDRs, or the anti-LRIG1 antibody can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of a polynucleotide that encodes the anti-LRIG1 antibody.

Expression of the vector can also be determined by examining the expressed anti-LRIG1 antibody. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

### Payload

In some embodiments, an anti-LRIG1 antibody further comprises a payload. In some cases, the payload comprises a small molecule, a protein or functional fragment thereof, a peptide, or a nucleic acid polymer.

In some cases, the number of payloads conjugated to the anti-LRIG1 antibody (e.g., the drug-to-antibody ratio or DAR) is about 1:1, one payload to one anti-LRIG1 antibody. In some cases, the ratio of the payloads to the anti-LRIG1 antibody is about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some cases, the ratio of the payloads to the anti-LRIG1 antibody is about 2:1. In some cases, the ratio of the payloads to the anti-LRIG1 antibody is about 3:1. In some cases, the ratio of the payloads to the anti-LRIG1 antibody is about 4:1. In some cases, the ratio of the payloads to the anti-LRIG1 antibody is about 6:1. In some cases, the ratio of the payloads to the anti-LRIG1 antibody is about 8:1. In some cases, the ratio of the payloads to the anti-LRIG1 antibody is about 12:1.

In some embodiment, the payload is a small molecule. In some instances, the small molecule is a cytotoxic payload. Exemplary cytotoxic payloads include, but are not limited to, microtubule disrupting agents, DNA modifying agents, or Akt inhibitors.

In some embodiments, the payload comprises a microtubule disrupting agent. Exemplary microtubule disrupting agents include, but are not limited to, 2-methoxyestradiol, auristatin, chalcones, colchicine, combretastatin, cryptophycin, dictyostatin, discodermolide, dolastain, eleutherobin, epothilone, halichondrin, laulimalide, maytansine, noscapinoid, paclitaxel, peloruside, phomopsin, podophyllotoxin, rhizoxin, spongistatin, taxane, tubulysin, vinca alkaloid, vinorelbine, or derivatives or analogs thereof.

In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268.

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 15 analog is cemadotin or tasidotin.

In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714, and 8,871,720.

In some embodiments, the payload comprises a DNA modifying agent. In some embodiments, the DNA modifying agent comprises DNA cleavers, DNA intercalators, DNA transcription inhibitors, or DNA cross-linkers. In some instances, the DNA cleaver comprises bleomycin A2, calicheamicin, or derivatives or analogs thereof. In some instances, the DNA intercalator comprises doxorubicin, epirubicin, PNU-159682, duocarmycin, pyrrolobenzodiazepine, oligomycin C, daunorubicin, valrubicin, topotecan, or derivatives or analogs thereof. In some instances, the DNA transcription inhibitor comprises dactinomycin. In some instances, the DNA cross-linker comprises mitomycin C.

In some embodiments, the DNA modifying agent comprises amsacrine, anthracycline, camptothecin, doxorubicin, duocarmycin, enediyne, etoposide, indolinobenzodiazepine, netropsin, teniposide, or derivatives or analogs thereof.

In some embodiments, the anthracycline is doxorubicin, daunorubicin, epirubicin, idarubicin, mitomycin-C, dactinomycin, mithramycin, nemorubicin, pixantrone, sabarubicin, or valrubicin.

In some embodiments, the analog of camptothecin is topotecan, irinotecan, silatecan, cositecan, exatecan, lurtotecan, gimatecan, belotecan, rubitecan, or SN-38.

In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120. In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In some embodiments, the payload comprises an Akt inhibitor. In some cases, the Akt inhibitor comprises ipatasertib (GDC-0068) or derivatives thereof.

In some embodiments, the payload comprises a polymerase inhibitor, including, but not limited to polymerase II inhibitors such as a-amanitin, and poly(ADP-ribose) polymerase (PARP) inhibitors. Exemplary PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, or 3-aminobenzamide.

In some embodiments, the payload comprises a detectable moiety. Exemplary detectable moieties include fluorescent dyes; enzymes; substrates; chemiluminescent moieties; specific binding moieties such as streptavidin, avidin, or biotin; or radioisotopes.

In some embodiments, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and antiandrogens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload comprises an immune modulator. Exemplary immune modulators include, but are not limited to, gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, xanthines, stem cell growth factors, lymphotoxins, hematopoietic factors, tumor necrosis factor (TNF) (e.g., TNFa), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “Si factor,” erythropoietin and thrombopoietin, or a combination thereof.

In some embodiments, the payload comprises an immunotoxin Immunotoxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

In some instances, the payload comprises a nucleic acid polymer. In such instances, the nucleic acid polymer comprises short interfering nucleic acid (siNA), short interfering RNA (siRNA), double-stranded RNA (dsRNA), micro-RNA (miRNA), short hairpin RNA (shRNA), an antisense oligonucleotide. In other instances, the nucleic acid polymer comprises an mRNA, encoding, e.g., a cytotoxic protein or peptide or an apoptotic triggering protein or peptide. Exemplary cytotoxic proteins or peptides include a bacterial cytotoxin such as an alpha-pore forming toxin (e.g., cytolysin A from E. coli), a beta-pore-forming toxin (e.g., α-Hemolysin, PVL—panton Valentine leukocidin, aerolysin, clostridial Epsilon-toxin, Clostridium perfringens enterotoxin), binary toxins (anthrax toxin, edema toxin, C. botulinum C2 toxin, C spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2). In additional instances, the nucleic acid polymer comprises a nucleic acid decoy. In some instances, the nucleic acid decoy is a mimic of protein-binding nucleic acids such as RNA-based protein-binding mimics Exemplary nucleic acid decoys include transactivating region (TAR) decoy and Rev response element (RRE) decoy.

In some cases, the payload is an aptamer. Aptamers are small oligonucleotide or peptide molecules that bind to specific target molecules. Exemplary nucleic acid aptamers include DNA aptamers, RNA aptamers, or XNA aptamers which are RNA and/or DNA aptamers comprising one or more unnatural nucleotides. Exemplary nucleic acid aptamers include ARC19499 (Archemix Corp.), REG1 (Regado Biosciences), and ARC1905 (Ophthotech).

Nucleic acids in accordance with the embodiments described herein optionally include naturally occurring nucleic acids, or one or more nucleotide analogs or have a structure that otherwise differs from that of a naturally occurring nucleic acid. For example, 2′-modifications include halo, alkoxy, and allyloxy groups. In some embodiments, the 2′-OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids having a variety of different nucleotide analogs, modified backbones, or non-naturally occurring internucleoside linkages are utilized in accordance with the embodiments described herein. In some cases, nucleic acids include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, 0(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display improved properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Different nucleotide modifications and/or backbone structures may exist at various positions in the nucleic acid. Such modification include morpholinos, peptide nucleic acids (PNAs), methylphosphonate nucleotides, thiolphosphonate nucleotides, 2′-fluoro N3-P5′-phosphoramidites, 1′,5′-anhydrohexitol nucleic acids (HNAs), or a combination thereof.

### Conjugation Chemistry

In some instances, the payload is conjugated to an anti-LRIG1 antibody described herein by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910.

In some instances, the payload is conjugated to an anti-LRIG1 antibody described herein by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the payload is conjugated to an anti-LRIG1 antibody described herein by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the payload is conjugated to an anti-LRIG1 antibody described herein by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some instances, the enzyme-catalyzed process comprises microbial transglutaminase (mTG). In some cases, the payload is conjugated to the anti-LRIG1 antibody utilizing a microbial transglutaminase catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some instances, the payload is conjugated to an anti-LRIG1 antibody by a method as described in PCT Publication No. WO2014/140317, which utilizes a sequence-specific transpeptidase and is hereby expressly incorporated by reference in its entirety.

In some instances, the payload is conjugated to an anti-LRIG1 antibody described herein by a method as described in U.S. Patent Publication Nos. 2015/0105539 and 2015/0105540.

### Linker

In some instances, a linker described above comprises a natural or synthetic polymer, consisting of long chains of branched or unbranched monomers, and/or cross-linked network of monomers in two or three dimensions. In some instances, the linker includes a polysaccharide, lignin, rubber, or polyalkylene oxide (e.g., polyethylene glycol).

In some instances, the linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is built up from monomers of another polymer. In some instances, the linker comprises polyalkylene oxide. In some instances, the linker comprises PEG. In some instances, the linker comprises polyethylene imide (PEI) or hydroxy ethyl starch (HES).

In some cases, the polyalkylene oxide (e.g., PEG) is a polydispers or monodispers compound. In some instances, polydispers material comprises disperse distribution of different molecular weight of the material, characterized by mean weight (weight average) size and dispersity. In some instances, the monodisperse PEG comprises one size of molecules. In some embodiments, the linker is poly- or monodispersed polyalkylene oxide (e.g., PEG) and the indicated molecular weight represents an average of the molecular weight of the polyalkylene oxide, e.g., PEG, molecules.

In some embodiments, the linker comprises a polyalkylene oxide (e.g., PEG) and the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some instances, the linker is a discrete PEG, optionally comprising from 2 to 60, from 2 to 50, or from 2 to 48 repeating ethylene oxide units. In some cases, the linker comprises a dPEG comprising about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some cases, the linker is a dPEG from Quanta Biodesign, LMD.

In some embodiments, the linker is a polypeptide linker. In some instances, the polypeptide linker comprises at least 2, 3, 4, 5, 6, 7, 8, 10, 15, 20, 25, 30, 35, 40, 45, 50, 60, 70, 80, 90, 100, or more amino acid residues. In some instances, the polypeptide linker comprises at least 2, 3, 4, 5, 6, 7, 8, or more amino acid residues. In some instances, the polypeptide linker comprises at most 2, 3, 4, 5, 6, 7, 8, or less amino acid residues. In some cases, the polypeptide linker is a cleavable polypeptide linker (e.g., either enzymatically or chemically). In some cases, the polypeptide linker is a non-cleavable polypeptide linker. In some instances, the polypeptide linker comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some instances, the polypeptide linker comprises a peptide such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the polypeptide linker comprises L-amino acids, D-amino acids, or a mixture of both L- and D-amino acids.

In some instances, the linker comprises a homobifuctional linker. Exemplary homobifuctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3¹-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[(β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMB s), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3¹-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such as1-(p-Azidosalicylamido)-4-(iodoacetamido)butane (AsIB), N-[4-(p-azidosalicylamido)butyl]-3-′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido)butylamine (AsBA), and arginine-reactive and photoreactive cross-linkers such as p-azidophenyl glyoxal (APG).

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker comprises PAMAM dendrimers.

In some embodiments, the linker is a traceless linker or a linker in which after cleavage does not leave behind a linker moiety (e.g., an atom or a linker group) to the antibody or payload. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

### Pharmaceutical Compositions

In some embodiments, an anti-LRIG1 antibody disclosed herein is further formulated as a pharmaceutical composition. In some instances, the pharmaceutical composition is formulated for administration to a subject by multiple administration routes, including but not limited to, parenteral (e.g., intravenous, subcutaneous, intramuscular, intraarterial, intradermal, intraperitoneal, intravitreal, intracerebral, or intracerebroventricular), oral, intranasal, buccal, rectal, or transdermal administration routes. In some instances, the pharmaceutical composition describe herein is formulated for parenteral (e.g., intravenous, subcutaneous, intramuscular, intraarterial, intradermal, intraperitoneal, intravitreal, intracerebral, or intracerebroventricular) administration. In other instances, the pharmaceutical composition describe herein is formulated for oral administration. In still other instances, the pharmaceutical composition describe herein is formulated for intranasal administration.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical compositions further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical compositions further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

### Therapeutic Regimens

In some embodiments, the anti-LRIG1 antibodies disclosed herein are administered for therapeutic applications. In some embodiments, the anti-LRIG1 antibody is administered once per day, twice per day, three times per day or more. The anti-LRIG1 antibody is administered daily, every day, every alternate day, five days a week, once a week, every other week, two weeks per month, three weeks per month, once a month, twice a month, three times per month, or more. The anti-LRIG1 antibody is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the anti-LRIG1 antibody is given continuously; alternatively, the dose of the anti-LRIG1 antibody being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, can be reduced, as a function of the symptoms, to a level at which the improved disease, disorder, or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an anti-LRIG1 antibody as disclosed herein, host cells for producing one or more antibodies described herein, and/or vectors comprising nucleic acid molecules that encode the antibodies described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1. Identification of LRIG1-Binding Antibodies with and without LRIG1-VISTA Blocking Activity

To identify LRIG1-binding antibodies with the capacity to block the assembly of LRIG1 and VISTA, an immunization campaign was run in mice. Balb/C, FVB, and CD-1F mice were inoculated at 7 day intervals with 50 ug of an isolated LRIG1-extracellular domain protein fused to a linker-spaced 6-histidine tag, LRIG1-ECD-His, (R&D systems, catalog #8504-LR) in combination with a TLR agonist adjuvant mix (50 μg MPL, 20 μg CpG, 10 μg Poly(I:C) and 10 μg R848) for 3 repetitions, followed by an inoculation with 50 ug of LRIG1-ECD-His alone administered subcutaneously to the inguinal, back of the neck and base of the tail sites as well as hock and intraperitoneal sites Animals were sacrificed in accordance with IACUC protocol and spleen, femurs, and lymph nodes (axillary, accessory axillary, mediastinal, superficial inguinal, iliac, sacral and popliteal) were harvested. A single cell suspension of immunized lymph node (LN), spleen and bone marrow cells were obtained using 2 sterile frosted glass slides in a tissue culture petri dish with 15 mL DMEM. Bone marrow was extracted from femurs via end-cap flushing with a 5 mL syringe fitted with an 18-gauge needle. Cells from 3 animals were pelleted with 5 minutes of centrifugation at 1200 RPM, resuspended in 10 mL of DMEM (GIBCO 10564-011) and nucleated cells were enumerated by hemocytometer count. Cells were pelleted at 1200 RPM and were resuspended in SC-Buffer (PBS, 2% FBS and 1 mM EDTA), and plasma cells were isolated with an EasySep™ Mouse CD138 Positive Selection Kit (StemCell Technologies) with the manufacturer recommended protocol. Enriched CD138-positive cells were pelleted with 5 minutes of centrifugation at 1200 RPM, resuspended in 50 mL electrofusion buffer (Eppendorf 940-00-220-6) and were enumerated. Separately, SP2/0-mIL6 myeloma cells (ATCC CRL2016) were pelleted with 5 minutes of centrifugation at 1200 RPM, resuspended in 50 mL electrofusion buffer and were enumerated. Myeloma cells and CD138-positive plasma cells were combined at a 1:1 ratio, volume was expanded to 50 mL with electrofusion buffer, cells were pelleted with 5 minutes of centrifugation at 1200 RPM and supernatant was discarded. After a repeated step of washing and pelleting in electrofusion buffer, cells were resuspended in electrofusion buffer to a concentration of 10×10\hat{ }6 cells/ml, up to 9 mL of cell suspension was added to a BTX electrofusion chamber, and cells were fused with an 800V electrofusion protocol. Fused cells were rested for 5 minutes, transferred to a tissue culture dish containing 40 mL medium MM (DMEM, 15% FBS, 1% glutamax and 1% Pen/Strep), incubated for 1 hour at 37 C, 8% CO2, resuspended with a pipette, pelleted with 5 minutes of centrifugation at 1200 RPM, resuspended in ClonaCell HY Liquid HAT Selection Medium (StemCell Technologies), and plated in 96-well tissue culture flat bottomed plates. After 10 days, supernatants were sampled and evaluated for binding to isolated LRIG1 ECD peptide by ELISA. 50 μl of 5 ug/ml streptavidin diluted in PBS was added to each well of 96-well plates (Nunc-Immuno MaxiSorp 439454) incubated overnight at 2-8° C., supernatant was discarded, and 50 μl of 0.45 ug/mL biotin-LRIG1 ECD (R&D systems Cat 8504, biotinylated in house) resuspended in diluent (PBS with 0.5% BSA) was added to each well for 45 minutes, supernatant was discarded and plates were washed with phosphate buffered saline (PBS) with 0.05% Tween20. 50 μl of 1:5 dilution of hybridoma supernatant in diluent was added to each well for 1 hour, followed by 5 successive 300 μl washes with PBS/0.05% Tween20, after which a 1:3000 dilution of goat anti-mouse Fc-specific antibody conjugated to horseradish peroxidase (Novex A16090) in 50 μl of diluent was added to each well for 1 hour followed by 5 successive 300 μl washes with PBS/0.05% Tween20. Following washing, 50 μl of ABTS (Novex #00-202-4) was added to each well for 20-30 minutes, prior to readout on a spectrophotometer (Molecular Devices) at absorbance of 405 nm.

Positively scoring wells were evaluated for the ability to block association of LRIG1 and VISTA. To identify LRIG1-targeted antibodies with the ability to block the interaction of LRIG1 and VISTA, purified LRIG1 and VISTA proteins were incubated in the presence of LRIG1-immunization hybridoma supernatants described above, or without antibody, and protein interaction was evaluated by ELISA. Purified human LRIG1 extracellular domain fused to a HIS tag (R&D Systems) was diluted in phosphate buffered saline (PBS) (Corning) to a concentration of 3 μg/ml and 100 μl was added to each well of a 96-well ELISA plate (Thermo Fisher, 44-2404-21). After incubating the plate at 4° C. overnight, the plate was washed three times with 300 μl of PBS with 0.05% TWEEN (VWR) (PBST) per well. The plate was then blocked for an hour with 200 μl of 2% bovine serum albumin (BSA) (Sigma) in PBST per well at room temperature with gentle rocking. Thereafter, the 2% BSA in PBST was removed and 50 μl of hybridoma supernatant diluted to 3.3 ug/mL was added to the wells. The plate was incubated for 10 minutes at room temperature with gentle rocking. Afterwards, 100 ul of 50 nM oligomerized VISTA in 100 ul PBS buffer was added per well. VISTA oligomerization was performed by Klickmer formation. Briefly, 5 nM Klickmer (Immudex) was incubated with 200 nM hVISTA-Fc-Avi-Biotin in PBS and incubated for one hour. The plate was incubated for an hour at room temperature with gentle rocking. Thereafter, the plate was washed three times with 300 μl of PBST per well, and 100 μl of avidin-HRP (1:1000) (Jackson ImmunoResearch) was then added to each well and the plate was incubated at room temperature for 30 minutes with gentle rocking. Thereafter, the plate was washed three times with 300 μl of PBST per well. 100 μl of TMB substrate (Fisher Scientific, 34029) was then added to each well. The reaction was stopped with 50 μl of 1 M HCl (VWR) per well. The plate was read using a spectrophotometer (Molecular Devices) at absorbance of 450 nm. Percent blockade of LRIG1-VISTA interaction was calculated as the fraction of signal obtained in each experimental samples of the no antibody sample less background signal.

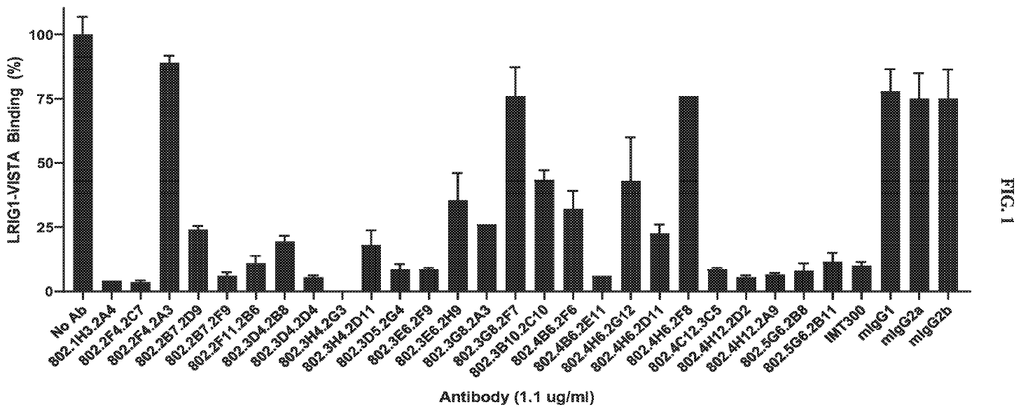

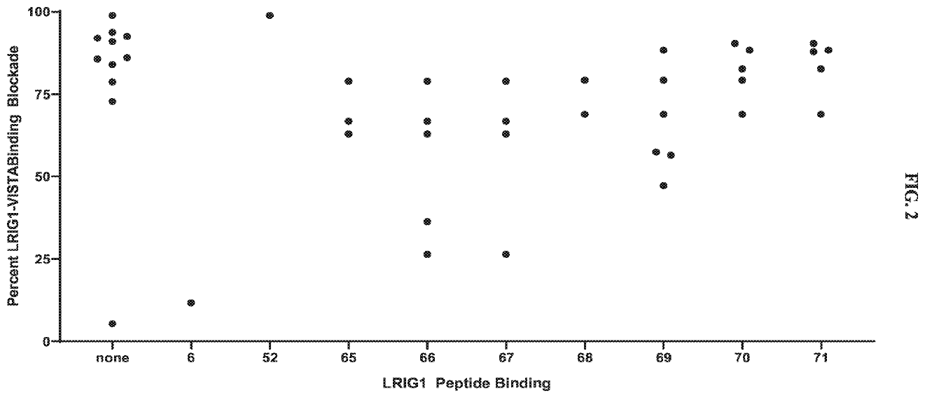

Several hLRIG1-binding antibodies with the ability to block the binding of LRIG1 to VISTA at a concentration of 1.1 ug/ml were identified, depicted in FIG. 1, including 802.3H4.2G3, which inhibited LRIG1-VISTA binding by 99%. Similarly, monoclonal antibodies 802.4H12.2D2, 802.4H12.2C3, 802.2F4.2C7, 802.1H3.2A4, 802.4B6.2E11, and 802.4H12.2A9 with the capacity to inhibit LRIG1-VISTA binding by 90%-99% were identified. Additionally, monoclonal antibodies with the capacity to inhibit LRIG1-VISTA binding by 80%-90% were identified, 802.3D4.2D4, 802.4C12.3C5, 802.2F11.2B6, 802.2B7.2D9, and 802.2B7.2F9 were identified, with IMT300 also blocking LRIG1-VISTA interaction in this range. Further, monoclonal antibodies with the capacity to inhibit LRIG1-VISTA binding by 50-80% were identified, 802.5G6.2B11, 802.3D5.2G4, 802.4B6.2F6, 802.5G6.2B8, 802.3E6.2F9, 802.3H4.2D11, 802.3E6.2H9, 802.4H6.2D11, and 802.3B10.2C10. Finally, monoclonal antibodies with the capacity to inhibit LRIG1-VISTA binding by 20% or less were identified, including 802.3G8.2F7 and 802.2F4.2A3.

### Example 2. LRIG1-Targeted Antibodies with and without VISTA-LRIG1 Blocking Activity Bind to Distinct Epitopes of LRIG1

To identify the epitopes to which LRIG1 antibodies with and without VISTA-LRIG1 blocking activity bound, a library of 20 amino acid peptides representing portions of LRIG1 was produced, and the ability to bind LRIG1 antibodies was evaluated by ELISA. At least 2 ug/ml of hLRIG1 peptide in 50 μl of PBS or 0.1 ug/ml of full-length human LRIG1 protein (R&D Systems) in 100 μl of PBS was added to the wells of a 96-well ELISA plate (Thermo Fisher, 44-2404-21). After incubating the plate at 4° C. overnight, the plate was washed three times with 300 μl of PBST per well. The plate was then blocked for an hour with 200 μl of 2% BSA in PBST per well at room temperature with gentle rocking. Thereafter, the 2% BSA in PBST was removed and 100 μl of 0.1 ug/ml of antibody in 2% BSA in PBST was added to the wells. The plate was incubated for an hour at room temperature with gentle rocking and then washed three times with 300 μl of PBST per well. Afterwards, 100 μl of anti-mouse IgG-HRP (1:4000) (Jackson ImmunoResearch) or anti-rat IgG HRP (1:4000) (Jackson ImmunoResearch) was added to the wells. The plate was incubated for 30 minutes at room temperature with gentle rocking and then washed three times with 300 μl of PBST per well. 100 μl of TMB substrate (Fisher Scientific, 34029) was then added to each well. The reaction was stopped with 50 μl of 1 M HCl (VWR) per well. The plate was read using a plate reader (Molecular Devices) at absorbance of 450 nm, and signal greater than 0.25 OD was considered evidence of peptide binding.

Binding of LRIG1-binding antibodies to the peptide array was observed at multiple locations, with the majority of binding observed in peptides 65-71 (see FIG. 2 and FIG. 4). Many antibodies with the strongest LRIG1-VISTA blocking activity failed to bind any peptide significantly, the latter observation indicating that these antibodies likely bind to a non-linear epitope. Antibody 802.2H4.2G3, which blocked LRIG1-VISTA association by 99.8% at 0.37 ug/ml was observed to bind peptide 52, corresponding to LRIG1 545-654. Accordingly, binding to this peptide sequence may be useful in the identification of antibodies with the ability to disrupt the interaction of LRIG1 and VISTA. Antibodies with somewhat less, but still strong, LRG1-VISTA blocking activity bound either to C-terminal peptides 70 and or 71 (802.4H12.2A9, 802.3D4.2D4, 802.2B7.2F9, and IMT300) indicating that antibody binding to peptides 70 and 71 is useful in the ability to predict the ability of a monoclonal antibody to disrupt LRIG1-VISTA interaction. Further, antibodies with more modest to poor ability to block LRIG1-VISTA binding were observed to bind 65, 66, 67, 68, and/or 69, (802.5G6.2B11, 802.3D5.2G4, 802.5G6.2B8, 802.3E6.2F9, 802.3H4.2D11, 802.3E6.2H9, 802.4H6.2D11, 802.3G8.2A3, and 802.4H6.2G12) likely suggesting that this region is adjacent to a region of LRIG1 that is important for LRIG1-VISTA binding, and suggests that these peptides are useful to identify antibodies with somewhat reduced activity in blocking the assembly of LRIG1 and VISTA. Finally, an antibody with poor LRIG1-VISTA blocking activity, 802.3G8.2F7, was observed to bind the N-terminal peptide 6. Collectively, these data support the capability of C-terminal peptides of LRIG1, especially peptides 70 and 71, to prospectively identify antibodies with the capacity to block LRIG1-VISTA binding.

### Example 3. LRIG1-VISTA Antibodies with Blocking Activity Compete for Binding to LRIG1

To determine whether LRIG1 binding antibodies with LRIG1-VISTA blocking activity bind to the same or overlapping regions of the LRIG1 molecule, antibody binning assays were performed to assess the ability of antibodies to bind simultaneously bind LRIG1 Amine-reactive probes were loaded onto a Gator biosensor (Probe Life, Palo Alto, Calif.), equilibrated in dH20 for 60 seconds, dipped into 100 μl EDC 0.2M/NHS 0.05M activation buffer for 30 seconds, then dipped into a solution of 20 ug/ul human LRIG1-His in 10 mM NaOAc buffer, pH 5 until binding was saturated and quenched in 1M ethanolamine pH 8.5 for 300 seconds. Following LRIG1-His loading, tips were dipped in 20 ug/mL saturating antibody, then successively dipped into 5 ug/mL competing antibody.

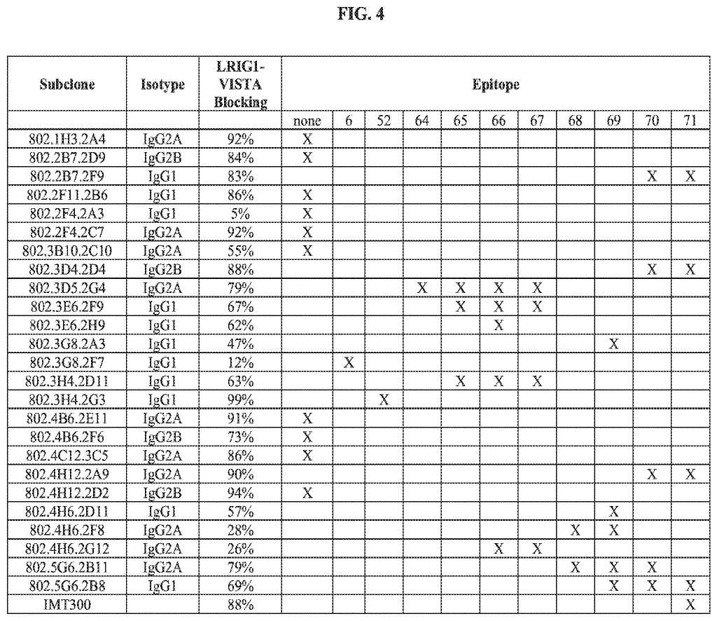

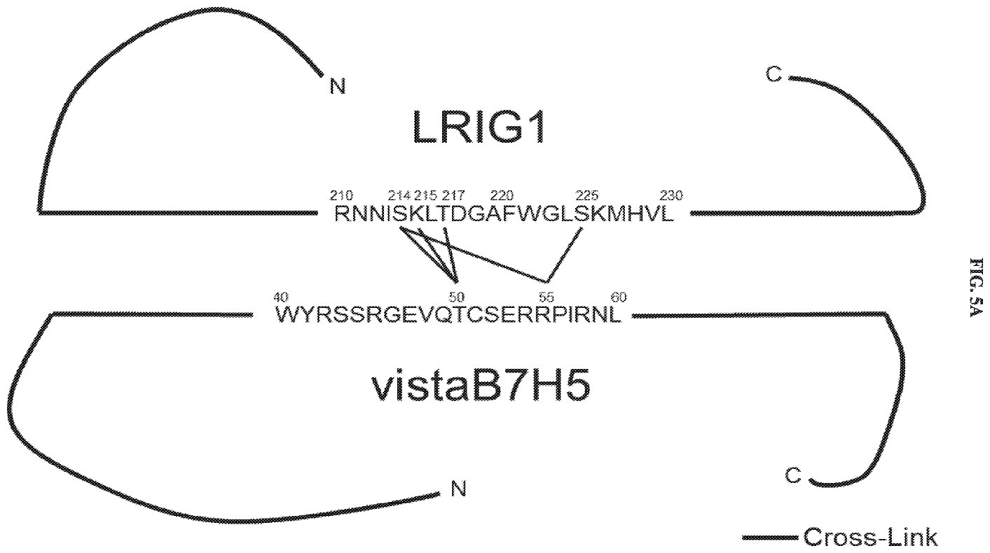

As depicted in FIG. 3, saturation of hLRIG1-His tips with any individual antibody prevented binding with the same antibody in a competition study. Competition between pairs of distinct antibodies revealed several antibodies which did not compete with any other antibody for binding, IMT300, 802.3E6.2F9, 802.2F4.2A3, 802.2B7.2D9, 802.1H3.2A4, 802.3H4.2G3, 802.4H12.2D2, and 802.486.2F8, comprising bins 1, 2, 3, 4, 5, 6, 7, and 11, respectively. Importantly, the antibody with the strongest ability to block LRIG1-VISTA assembly, 802.3H4.2G3, defines bin6, whereas the second most potent blocking antibody, 802.4H12.2D2, defines bin7. Accordingly, both bin6 and bin7 would be useful to identify other antibodies with the capacity to block LRIG1 and VISTA binding. Among antibodies which did exhibit competitive binding profiles, 802.3D4.2D4, 802.4H12.2A9, 802.5G6.2B8, and 802.2B7.2F9 were mutually competitive for binding to LRIG1, defining bin8. Each of these antibodies also bound to peptides 70 and 71, suggesting that the shared amino acid sequence comprising these sequences are essential for the definition of this bin. Additionally, these antibodies all exhibited moderate ability to block LRIG1 and VISTA assembly, indicating that competitive binding by an antibody to LRIG1 with antibodies in bin8 would be useful in the identification of antibodies which would have the capacity to moderately block LRIG1 and VISTA binding. Similarly, antibodies 802.5G6.2B11 and 802.3G8.2A3 exhibited mutually competitive binding to LRIG1, defining bin9. These antibodies bound peptide 69, suggesting that the amino acid sequence comprising this sequence is essential for the definition of this bin. Additionally, these antibodies all exhibited modest ability to block LRIG1 and VISTA assembly, indicating that competitive binding by an antibody to LRIG1 with antibodies in bin9 would be useful in the identification of antibodies which would have the capacity to modestly block LRIG1 and VISTA binding. Further, antibodies 802.4B6.2E11 and 802.4C12.3C5 exhibited mutually competitive binding to LRIG1, defining bin10. Neither of these antibodies significantly bound any LRIG1 peptide, suggesting that the binding epitope for this bin is non-linear. Antibodies in bin 10 exhibited moderate ability to block LRIG1 and VISTA assembly, indicating that competitive binding by an antibody to LRIG1 with antibodies in bin10 would be useful in the identification of antibodies which would have the capacity to moderately block LRIG1 and VISTA binding. Finally, some antibodies failed to bind to LRIG1 in this format, suggesting that particularities of this format are unfavorable to antibody binding, including 802.2F4.2C7, 802.2F11.2B6, 802.4H6.2G12, and 802.3G8.2F7.

### Example 4. LRIG1 Expression Assay

Flow cytometry is used to detect LRIG1 expression on human peripheral blood mononuclear cell (PBMC). Human PBMC is seeded on plates coated with hCD3 and hCD28 (Biolegend) for 5 days for activation. Activated or fresh PBMC is blocked with 200 μg/ml hIgG for 10 minutes on ice, followed by incubation with sheep anti-hLRIG1 polyclonal antibody (R&D Systems) or an isotype control antibody for 20 minutes on ice, followed by incubation with anti-sheep IgG APC antibody (Jacksonlmmuno Research) for 20 minutes on ice. After wash, stained cells are analyzed using MACSquant Analyzer 10 (Miltenyi Biosci).

### Example 5. LRIG1 Function-Mixed Lymphocyte Reaction (MLR)

Human M2 macrophages from one donor are mixed with human CD4 T cells from another donor and are treated with 10 ug/ml control IgG, hPD1 blocking antibody EH12 (BD bioscience), hLRIG1 mAb IMT300 (mab4), or the combination of hPD1 and LRIG1 antibodies for 8 days. Secreted IFNgamma (IFNy) is detected with an ELISA kit from eBioscience.

### Example 6. Identification of VISTA-LRIG1 Binding Surface

To identify the residues mediating the interaction between LRIG1 and VISTA, a crosslinked mass spectroscopy approach was used. 5 μl of purified 3.2 μM LRIG1 and 5 μl of purified 0.6 μM VISTA were mixed and were submitted to cross-linking with a K200 MALDI MS analysis kit (CovalX). 9 μl of the mixture was mixed with 1 μl of K200 Stabilizer reagent (2 mg/ml) and incubated at room temperature. After the incubation time (180 minutes) the samples were prepared for MALDI analysis as for Control experiments. The samples were analyzed by High-Mass MALDI analysis immediately after crystallization. For the analysis, the following parameters have been applied: Mass Spectrometer: Linear and Positive mode, Ion Source 1: 20 kV, Ion Source 2: 17 kV, Lens: 12 kV, Pulse Ion Extraction: 400 ns HM4: Gain Voltage: 3.14 kV, Acceleration Voltage: 20 kV. Crosslinked LRIG1-VISTA products were identified with MH+=207.154 kDa. Samples were digested with Trypsin, chymotrypsin, ASPN-N, Elastase, or Thermolysin and crosslinked peptides with both LRIG1 and VISTA amino acid sequences were determined.

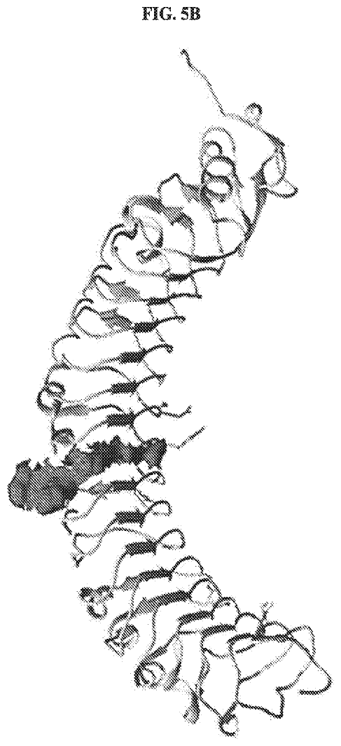

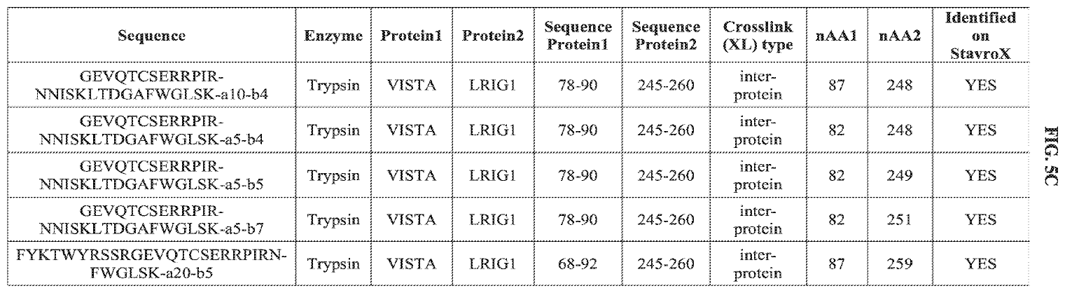

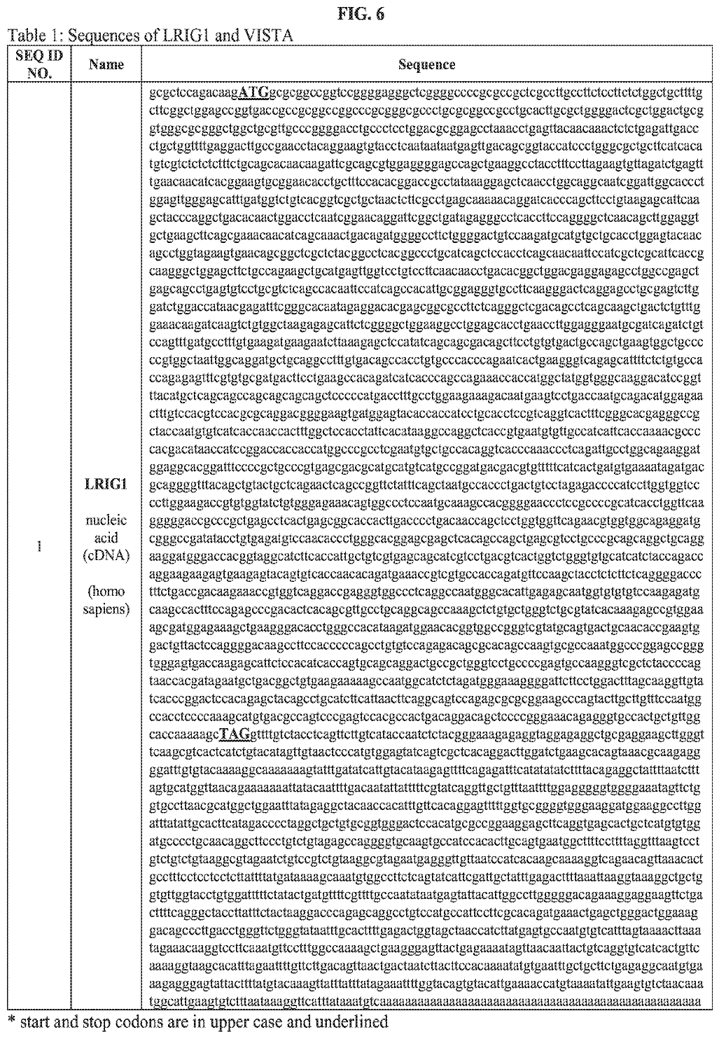

As depicted in FIGS. 5A-5C, residues in the vicinity of LRIG1 amino acids 245-260 were found to be crosslinked to residues in the vicinity of VISTA amino acids 68-92. These amino acids are located on exposed regions of each molecule, suggesting that these regions are involved in the protein-protein interaction of LRIG1 and VISTA. It is notable that the LRIG1 binding interface at amino acids 246-260 determined by MALDI-MS is distinct from the epitope bound by the LRIG1-VISTA blocking antibodies 802.1H3.2A4, 802.2B7.2D9, 802.2B7.2F9, 802.2F11.2B6, 802.2F4.2A3, 802.2F4.2C7, 802.3B10.2C10, 802.3D4.2D4, 802.3D5.2G4, 802.3E6.2F9, 802.3E6.2H9, 802.3G8.2A3, 802.3G8.2F7, 802.3H4.2D11, 802.3H4.2G3, 802.4B6.2E11, 802.4B6.2F6, 802.4C12.3C5, 802.4H12.2A9, 802.4H12.2D2, 802.4H6.2D11, 802.4H6.2F8, 802.4H6.2G12, 802.5G6.2B11, and 802.5G6.2B8. The binding of these antibodies may induce a conformational shift that causes a structural rearrangement, thereby impacting binding. Identification of a distinct binding interface mediated by of LRIG1 amino acids 245-260 suggests that antibodies which bind a region other than defined by peptide 52 could also disrupt the interaction of LRIG1 and VISTA.

### Example 7. LRIG1-VISTA Blockade Reduces Tumor Growth in a Humanized Mouse Tumor Model

To evaluate the utility of LRIG1-VISTA blockade in the setting of cancer, mice engrafted with a human immune system and bearing human SCLC tumors were employed. All animal studies were conducted in compliance with the U.S. Department of Agriculture's Animal Welfare Act (9 CFR Parts 1, 2 and 3) as applicable and were covered by an IACUC approved animal protocol. Briefly, NOD.Cg-Prkdc^{scid} Il2rg^{tm1Sug} Tg(SV40/HTLV-IL3, CSF2)10-7Jic/JicTac mice, also known as NOG-EXL mice (Taconic), were engrafted with human CD34+ hematopoietic stem cells, and 50,000 human small cell lung cancer (SCLC) patient derived xenograft (PDX) model LU5173 tumor cells mixed with an equal volume of Cultrex ECM (Trevigen) in 100 μl total volume were subcutaneously injected into the rear flank with a chilled 1 ml Luer-lok syringe fitted with a 26G 7/8 (0.5 mm×22 mm) needle. Animals were monitored weekly for palpable tumors, or any changes in appearance or behavior and daily monitoring was conducted for mice showing any signs of morbidity or mortality. Tumor volume was calculated using the following equation: (longest diameter*shortest diameter²)/2. When average tumor volume reached 60-100 mm³, 12 mice were randomly assigned to the respective treatment groups receiving either A) HuIgG4 control antibody, BIW×3 weeks at 10 mg/kg; B) Anti-PD1 OPDIVO antibody, BIW×3 weeks at 10 mg/kg or; C) anti-LRIG1 antibody BIW×3 weeks at 10 mg/kg.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An antigen binding polypeptide, wherein the polypeptide exhibits specific binding to LRIG1 protein (SEQ ID NO: 2), and wherein the polypeptide binds to an epitope present on one or more regions of the LRIG1 protein selected from a group consisting of any amino acid sequence from amino acid residues from position 1 to 564 or position 655 to 1093 from N terminus to C terminus of the LRIG1 protein.

2.-13. (canceled)

14. The antigen binding polypeptide of claim 1, wherein the polypeptide disrupts LRIG1-VISTA interaction, and wherein the polypeptide binds to an epitope of LRIG1 in a region from SEQ ID NOs: 69 to 75.

15. The antigen binding polypeptide of claim 1, wherein the polypeptide binds to an epitope present within the region of LRIG1 defined by Peptide 65 (SEQ ID NO: 69).

16. The antigen binding polypeptide of claim 1, wherein the polypeptide binds to an epitope present within the region of LRIG1 defined by Peptide 66 (SEQ ID NO: 70).

17. The antigen binding polypeptide of claim 1, wherein the polypeptide binds to an epitope present within the region of LRIG1 defined by Peptide 67 (SEQ ID NO: 71).

18. The antigen binding polypeptide of claim 1, wherein the polypeptide binds to an epitope present within the region of LRIG1 defined by Peptide 68 (SEQ ID NO: 72).

19. The antigen binding polypeptide of claim 1, wherein the polypeptide binds to an epitope present within the region of LRIG1 defined by Peptide 69 (SEQ ID NO: 73).

20. The antigen binding polypeptide of claim 1, wherein the polypeptide binds to an epitope present within the region of LRIG1 defined by Peptide 70 (SEQ ID NO: 74).

21. The antigen binding polypeptide of claim 1, wherein the polypeptide binds to an epitope present within the region of LRIG1 defined by Peptide 71 (SEQ ID NO: 75).

22. The antigen binding polypeptide of claim 1, wherein the antigen binding polypeptide comprises a full-length antibody or a fragment thereof.

23. The antigen binding polypeptide of claim 1, wherein the antigen binding polypeptide comprises a bispecific antibody or a binding fragment thereof.

24. The antigen binding polypeptide of claim 1, wherein the antigen binding polypeptide comprises a monovalent Fab′, a divalent Fab2, a single-chain variable fragment (scFv), a diabody, a minibody, a nanobody, a single-domain antibody (sdAb), or a camelid antibody or binding fragment thereof.

25. (canceled)

26. The antigen binding polypeptide of claim 1, wherein the antigen binding polypeptide comprises an IgG framework, an IgG1 framework, an IgG2 framework, or an IgG4 framework.

27. (canceled)

28. The antigen binding polypeptide of claim 1, wherein the antigen binding polypeptide comprises a k_{D} of less than 30 nM.

29. The antigen binding polypeptide of claim 1, wherein the antigen binding polypeptide comprises a humanized antibody.

31.-52. (canceled)

53. An antigen binding polypeptide, wherein the polypeptide exhibits specific binding to LRIG1 protein such that upon binding said polypeptide reduces an interaction between LRIG1-VISTA by (i) at least 79%, wherein said antibody is not IMT300 or (ii) a greater degree than IMT300.

54. (canceled)

55. A method of disrupting an interaction between VISTA and LRIG1, comprising:
contacting a plurality of cells comprising a LRIG1-expressing cell, a VISTA-expressing cell, or a combination thereof with the antigen binding polypeptide of claim 1.

56.-70. (canceled)

71. A method of modulating an immune response in a subject, comprising:
administering to the subject an antigen binding polypeptide that specifically binds to an LRIG1 protein (SEQ ID NO: 2).

72.-91. (canceled)

92. A pharmaceutical composition comprising the antigen binding polypeptide of claim 1 and at least one pharmaceutically acceptable carrier, excipient, diluent, or adjuvant.

93. The antigen binding polypeptide of claim 1, wherein the antigen binding polypeptide comprises at least one heavy chain CDR1 selected from the group consisting of SEQ ID NOs: 84, 87, 90, 93, 96, 99, 102, 105, 108, 111, 114, 117, 120, 123, 126, 129, 134, 138, 143, and 242.

94. The antigen binding polypeptide of claim 1, wherein the antigen binding polypeptide comprises at least one heavy chain CDR2 selected from the group consisting of SEQ ID NOs: 85, 88, 91, 94, 97, 100, 103, 106, 109, 112, 115, 118, 121, 124, 127, 130, 132, 136, 139, 141, 142, 144, and 243.

95. The antigen binding polypeptide of claim 1, wherein the antigen binding polypeptide comprises at least one heavy chain CDR3 selected from the group consisting of SEQ ID NOs: 86, 89, 92, 95, 98, 101, 104, 107, 110, 113, 116, 119, 122, 125, 128, 131, 133, 135, 137, 140, 145, and 244.

96. The antigen binding polypeptide of claim 1, wherein the antigen binding polypeptide comprises at least one light chain CDR1 selected from the group consisting of SEQ ID NOs: 149, 152, 154, 157, 163, 166, 168, 171, 172, 173, 175, 178, 181, 183, 187, 246, 251, and 253.

97. The antigen binding polypeptide of claim 1, wherein the antigen binding polypeptide comprises at least one light chain CDR2 selected from the group consisting of SEQ ID NOs: 150, 155, 158, 160, 164, 169, 176, 179, 185, 247, and 249.

98. The antigen binding polypeptide of claim 1, wherein the antigen binding polypeptide comprises at least one light chain CDR3 selected from the group consisting of SEQ ID NOs: 151, 153, 156, 159, 161, 162, 165, 167, 170, 174, 177, 180, 182, 184, 186, 248, 250, and 252.

99. The antigen binding polypeptide of claim 1, wherein the antigen binding polypeptide comprises 3 heavy chain CDRs selected from the group consisting of SEQ ID NOs: 81-145 and 242-245, and 3 light chain CDRs selected from the group consisting of SEQ ID NOs: 146-187 and 246-254.

100. (canceled)

101. The antigen binding polypeptide of claim 1, wherein the antigen binding polypeptide comprises a VH domain having a sequence with at least 80%, 85%, 90%, 95%, 96%, 97%, 98%, 99%, or 100% homology to SEQ ID NOs: 192-216.

102. The antigen binding polypeptide of claim 1, wherein the antigen binding polypeptide comprises a VL domain having a sequence with at least 80%, 85%, 90%, 95%, 96%, 97%, 98%, 99%, or 100% homology to SEQ ID NOs: 217-241.

103. (canceled)

104. (canceled)
